(19) United States
(12) Patent Application Publication
(10) Pub. No.: US 2025/0195694 A1
(43) Pub. Date: Jun. 19, 2025

(54) METHODS AND COMPOSITIONS FOR TREATING TNNT2 RELATED CARDIOMYOPATHY WITH A VIRAL VECTOR

(71) Applicants: University of Florida Research Foundation, Incorporated, Gainesville, FL (US); AavantiBio, Inc., Charlestown, MA (US)

(72) Inventors: Barry John Byrne, Gainesville, FL (US); Manuela Corti, Gainesville, FL (US); Widler Casy, Charlestown, MA (US); Adam Cockrell, Charlestown, MA (US)

(73) Assignees: University of Florida Research Foundation, Incorporated, Gainesville, FL (US); AavantiBio, Inc., Charlestown, MA (US)

(21) Appl. No.: 18/847,734

(22) PCT Filed: Mar. 18, 2023
(86) PCT No.: PCT/US2023/064673

§ 371 Date: Sep. 17, 2024

Related U.S. Application Data
(60) Provisional application No. 63/321,264, filed on Mar. 18, 2022.

(51) Int. Cl. A61K 48/00; C07K 14/47; C12N 15/113; C12N 15/86

(52) U.S. Cl. CPC A61K 48/0058; C07K 14/4716; C12N 15/113; C12N 15/86; C12N 2310/14; C12N 2310/531; C12N 2320/31; C12N 2750/14143; C12N 2800/22

(57) ABSTRACT

The present disclosure relates to compositions and methods for the treatment of cardiomyopathy. Several embodiments provided for herein relate to virally-mediated transfer of a gene to host cells to induce expression of an encoded polypeptide, protein or other product to host in order to ameliorate one or more symptoms of the cardiomyopathy. In several embodiments, the methods and compositions relate to recombinant adeno-associated virus particles encoding human TNNT2 in order to treat cardiomyopathies, including dilated cardiomyopathy or hypertrophic cardiomyopathy.

### PRIORITY

This application claims priority to U.S. Provisional Patent Application No. 63/321,264, filed Mar. 18, 2022, the entire contents of which is incorporated by reference herein.

### BACKGROUND

Cardiomyopathy represents a collection of diverse conditions of the heart muscle and is the second most common cause of heart disease in subjects and medical management of the secondary signs is the only therapeutic option. These diseases have many causes, symptoms, and treatments, and can affect people of all ages and races. When cardiomyopathy occurs, the normal muscle in the heart can thicken, stiffen, thin out, or fill with substances the body produces that do not belong in the heart muscle. As a result, the heart muscle's ability to pump blood is reduced, which can lead to irregular heartbeats, the backup of blood into the lungs or rest of the body, and heart failure. Cardiomyopathy can be acquired or inherited. The cause isn't always known but there is an increasing understanding of the genetic underpinnings of inherited forms of disease.

Gene transfer strategies have been shown to ameliorate heart disease.

### INCORPORATION BY REFERENCE OF MATERIAL IN SEQUENCE LISTING FILE

This application incorporates by reference the material in the Sequence Listing contained in the following XML file being submitted concurrently herewith: File name: U120270091WO00-SEQ-PRW.xml; created on Mar. 16, 2023 and 204,832 bytes in size.

### SUMMARY

Cardiomyopathy is a class of disease of heart muscle that adversely impacts the hearts ability to circulate blood through the cardiovascular system. Various types of cardiomyopathies exist, including dilated cardiomyopathy, hypertrophic cardiomyopathy, and restrictive cardiomyopathy. Cardiomyopathy in human populations is a major medical burden and treatment needs are currently unmet, despite cardiomyopathies in human populations being particularly desirable to treat.

Dilated cardiomyopathy (DCM) is one of the most common types of human cardiomyopathy, occurring mostly in adults 20 to 60. DCM affects the heart's ventricles and atria, the lower and upper chambers of the heart, respectively. Most forms of DCM are acquired forms from a number of causes that include coronary heart disease, heart attack, high blood pressure, diabetes, thyroid disease, viral hepatitis and viral infections that inflame the heart muscle. Alcohol abuse and certain drugs, such as cocaine and amphetamines, as well as at least two drugs used to treat cancer (doxorubicin and daunorubicin), can also lead to DCM. In addition, there are a number of genetic forms of DCM, including, but not limited to the DCM associated with Duchenne and Becker muscular dystrophies. In the case of certain forms of Becker muscular dystrophy, as well as in most cases of Duchenne muscular dystrophy, the cardiomyopathy can ultimately limit the patient's survival.

Hypertrophic cardiomyopathy (HCM) occurs when the walls of the heart muscle become abnormally thick. The increase in wall thickness may increase cardiac complications, as well as block or obstruct blood flowing in the heart.

Restrictive cardiomyopathy (RCM) is a condition leading to a stiffening of the chambers of the heart over time. While the heart's ability to contract remains largely unaffected, the cardiac muscle does not fully relax between beats of the heart. This restricts the ability of the ventricles to fill with blood and causes blood to back up in the circulatory system.

Heart function is critically dependent upon calcium-dependent signaling. During heart disease, malfunctioning of calcium channels within cardiac cells promotes calcium cycling abnormalities, further inhibiting heart function. Gene transfer strategies to reduce calcium cycling abnormalities are reported to ameliorate heart disease in small and large animal models, as well as in human clinical trials.

Disclosed herein are gene delivery approaches for treatment of human subjects with one or more types of cardiomyopathy or symptoms thereof.

Accordingly, some aspects of the present disclosure provide recombinant adeno-associated virus (rAAV) vectors for delivering transgenes into the heart of a subject. Such rAAV vectors may include, from 5′ to 3′, in order, a first adeno-associated virus (AAV) inverted terminal repeat (ITR) sequence, a promoter operably linked to the one or more transgene, and a second AAV inverted terminal repeat (ITR) sequence. In some embodiments, the rAAV vector includes, in addition to a promoter, a regulatory element which modifies expression, e.g., in a manner that provides physiologically relevant expression levels and/or restricts expression to a particular cell type or tissue. In some embodiments, the regulatory element comprises one or more of an enhancer, a 5′ untranslated region (UTR), and a 3′ UTR. In some embodiments, the rAAV vector also includes at least one polyadenylation signal (e.g., positioned 3′ of the transgene). In some embodiments, two transgenes are operably linked to the same single promoter. In some embodiments, each transgene is operably linked to a separate promoter. In some embodiments in which multiple transgenes are provided, the rAAV vector also includes at least one polyadenylation signal (e.g., positioned 3′ of two transgenes expressed from a single promoter or 3′ of one or both transgenes expressed from different promoters). Aspects of the disclosure provide recombinant adeno-associated virus (rAAV) nucleic acid vector for delivering two or more transgenes into the heart of a subject, wherein said vector comprises, from 5′ to 3′, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence.

In some embodiments, the therapeutic transgene is encoded by a polynucleotide having at least 85%, at least 90%, at least 95%, at least 96%, at least 97%, at least 98% or at least 99% sequence identity to the nucleotide sequence set forth as SEQ ID NO: 7-27, or SEQ ID NO: 53-71, or SEQ NO: 78-95, or SEQ NO: 104-121 arranged in sequence. In some embodiments, one or more of the transgenes of the present disclosure are naturally-occurring sequences. In some embodiments, one or more transgenes are engineered to be species-specific. In some embodiments, one or more transgenes are codon-optimized for expression in a species of interest, e.g., human. For example, in several embodiments, the therapeutic transgene (e.g., the TNNT2 transgene) are codon-optimized.

Further provided herein are rAAV particles containing the rAAV vectors disclosed herein, encapsidated in AAV capsids. Other aspects of the present disclosure include compositions containing the rAAV particles described herein. In several embodiments, such compositions may be administered to a subject for gene therapy for cardiomyopathy. In additional embodiments, such compositions may be administered to a subject for gene therapy for heart disease. In some embodiments, the heart disease causes heart failure in the subject.

The compositions of the present disclosure may be administered to the subject via different routes. In some embodiments, the composition is administered via intravenous injection into the subject. In some embodiments, the administration of the composition results in expression of the transgene (or, if multiple transgenes are used, expression of two or more transgenes) in the subject's heart. In various embodiments, the step of administering the composition results in improved cardiac function in the subject, such as improved cardiac function in the subject for more than 10 months. In some embodiments, administration results in improved cardiac function for more than 12 months, more than 14 months, more than 16 months, more than 17 months, more than 20 months, more than 22 months, or more than 24 months. In several embodiments, improved cardiac function is represented by an increase in left ventricular ejection fraction (LVEF). In several embodiments, the LVEF (as compared to a pre-therapy measurement) increases by at least about 1%, about 2%, about 3%, about 4%, about 5% or more (including any amount between those listed). In several embodiments, LVEF is measured by echocardiography. In some embodiments, administration results in improved cardiac physiology (e.g., structural features) for more than 12 months, more than 14 months, more than 16 months, more than 17 months, more than 20 months, more than 22 months, or more than 24 months. In several embodiments, the improved cardiac physiology is represented by a decrease in left ventricular wall thickness. In several embodiments, left ventricular wall thickness is reduced by at least about 1%, about 2%, about 3%, about 4%, about 5% or more (including any amount between those listed). In several embodiments, the left ventricular wall thickness is measured by cardiac magnetic resonance imaging (MRI) or transthoracic echocardiography (TTE).

In some embodiments, described herein are compositions comprising AAV vectors, virions, viral particles, and pharmaceutical formulations thereof, useful in methods for delivering genetic material encoding one or more beneficial or therapeutic product(s) to mammalian cells and tissues. The rAAV vectors, rAAV particles, or the composition comprising the rAAV particles of the present disclosure, may be used for gene therapy for heart diseases in a subject in need thereof, such as one or more types of cardiomyopathy.

Additionally, provided herein are compositions, as well as therapeutic and/or diagnostic kits that include one or more of the disclosed AAV compositions, formulated with one or more additional ingredients, or prepared with one or more instructions for their use.

In some embodiments, described herein is a nucleic acid comprising an expression construct comprising a human TNNT2 coding sequence, an optional silencing element, and an enhancer element, such as a CMV enhancer, operably linked to a promoter, wherein the expression construct is flanked on each side by an inverted terminal repeat sequence. In some embodiments, the silencing elements comprise an shRNA expression cassette. In some embodiments, the silencing elements comprise an shRNA sequence. In some embodiments, the human TNNT2 coding sequence is codon-optimized for expression in human cells. In some embodiments, the promoter comprises a cardiac specific promoter. In some embodiments, the promoter is TNNT2. In some embodiments, the promoter is MHCK9. In some embodiments, the promoter is MHCK7. In some embodiments, the promoter is CBA (Chicken β-Actin). In some embodiments, the promoter is CMV or mini CMV. In some embodiments, the promoter is a Desmin promoter. In some embodiments, the nucleic acid is a recombinant adeno-associated virus (rAAV) vector. In some embodiments, the nucleic acid is a single-stranded or self-complementary rAAV nucleic acid vector. In some embodiments, the rAAV particle is an AAV9 particle. In some embodiments, the rAAV particle is an rh74 particle. In some embodiments, the rAAV particle is an rh10 particle. In some embodiments, a composition comprising a plurality of rAAV particles is provided. In some embodiments, the plurality of rAAV particles may further comprise a pharmaceutically acceptable carrier.

In some embodiments, described herein is a nucleic acid comprising an expression construct comprising a human RBM20 coding sequence and an enhancer element, such as a CMV enhancer, operably linked to a promoter, wherein the expression construct is flanked on each side by an inverted terminal repeat sequence. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is non-native with respect to the human RBM20 coding sequence and/or non-native to the promoter. In some embodiments, described herein is a nucleic acid comprising an expression construct comprising a human RBM20 coding sequence, an enhancer element operably linked to a promoter, and an in silico designed consensus Kozak sequence, wherein the in silico designed consensus Kozak sequence enhances transgene expression in the heart, wherein the expression construct is flanked on each side by an inverted terminal repeat sequence, wherein the Kozak sequence is non-native with respect to the human RBM20 coding sequence and the promoter. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is native with respect to the human RBM20 coding sequence and/or native to the promoter. In several embodiments, the Kozak sequence is a synthetic sequence. In some embodiments, the human RBM20 coding sequence is codon-optimized for expression in human cells. In some embodiments, the promoter comprises a cardiac specific promoter. In some embodiments, the promoter is CBA (Chicken β-Actin), or a truncated chicken beta-actin (smCBA). In some embodiments, the nucleic acid is a recombinant adeno-associated virus (rAAV) vector. In some embodiments, the nucleic acid is a single-stranded or self-complementary rAAV nucleic acid vector. In some embodiments, the rAAV particle is an AAV9 particle. In some embodiments, the rAAV particle is an rh74 (or AAVrh74) particle. In some embodiments, the rAAV particle is an rh10 (or AAVrh10) particle. In some embodiments, a composition comprising a plurality of rAAV particles is provided. In some embodiments, the plurality of rAAV particles may further comprise a pharmaceutically acceptable carrier. In some embodiments, the rh74 particle comprises at least one capsid protein encoded by a polynucleotide having at least 85%, at least 90%, at least 95%, at least 96%, at least 97%, at least 98% or at least 99% sequence identity to the nucleotide sequence set forth as SEQ ID NO: 10, or a portion of SEQ ID NO: 10 (for example, SEQ ID NO: 10 encodes the rh74 VP1, VP2, and VP3 proteins—thus, in several embodiments, an rh74 particle according to embodiments disclosed herein comprises at least one capsid protein encoded by a polynucleotide having at least 85%, at least 90%, at least 95%, at least 96%, at least 97%, at least 98% or at least 99% sequence identity to a subpart of the nucleotide sequence of SEQ ID NO: 10). In some embodiments, the rh74 particle comprises an amino acid sequence having at least about 85%, at least 90%, at least 95%, at least 96%, at least 97%, at least 98% or at least 99% sequence identity to the amino acid sequence set forth as SEQ ID NO: 11, or a portion of SEQ ID NO: 11 (for example, SEQ ID NO: 11 is the amino acid sequence of rh74 VP1, VP2, and VP3 proteins-thus, in several embodiments, an rh74 particle according to embodiments disclosed herein comprises at least one capsid protein having at least 85%, at least 90%, at least 95%, at least 96%, at least 97%, at least 98% or at least 99% sequence identity to a subpart of the amino acid sequence of SEQ ID NO: 11). In some embodiments, the AAV9 particle comprises an amino acid sequence having at least about 85%, at least 90%, at least 95%, at least 96%, at least 97%, at least 98% or at least 99% sequence identity to the amino acid sequence set forth as SEQ ID NO: 12.

In some embodiments, a method of treating dilated cardiomyopathy or hypertrophic cardiomyopathy is described, the method comprising administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2, thereby treating the cardiomyopathy. In some embodiments, the rAAV is administered via intravenous injection.

In some embodiments, a method of treating cardiomyopathy is described, the method comprising administering a therapeutically effective amount of rAAV comprising a nucleic acid expression construct comprising a human TNNT2 coding sequence, an optional silencing element, each element operably linked to a promoter and optionally comprising and an enhancer element, wherein the expression construct is flanked on each side by an inverted terminal repeat sequence, and wherein said administration results in expression of a therapeutically effective amount of human TNNT2, thereby treating the cardiomyopathy.

In some embodiments, a method of treating cardiomyopathy is described, the method comprising administering a therapeutically effective amounts of (1) an optional silencing construct, e.g., an rAAV comprising a silencing construct, and (2) an rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2, thereby treating the cardiomyopathy. In some embodiments, the rAAV is administered via intravenous injection.

In some embodiments, the rAAV, e.g., comprising a TNNT2 coding sequence and/or the silencing construct are administered via intravenous injection. In some embodiments, between about 1×10¹³ and about 1×10¹⁴ rAAV vector genomes are administered. In some embodiments, at 20%, at least 30%, at least 40%, or at least 50% of cardiomyocyte cells are transduced when the rAAV vector genomes are administered. In some embodiments, at 20%, at least 30%, at least 40%, or at least 50% of cardiomyocyte cells are transduced when between about 1×10¹³ and about 1×10¹⁴ rAAV vector genomes are administered.

Also described herein is a method of inducing increased expression of human TNNT2 in a target cell, comprising contacting a target cell with a plurality of rAAV particles comprising a nucleic acid expression construct comprising a functional human TNNT2 coding sequence, an optional silencing element, and optionally an enhancer element operably linked to a promoter, wherein the expression construct is flanked on each side by an inverted terminal repeat sequence, and wherein said contacting results in the target cell increasing expression of functional human TNNT2 as compared to prior to the contacting, thereby increasing the expression of functional human TNNT2.

Also described herein is a method of inducing increased expression of human TNNT2 in a target cell, comprising contacting a target cell with a plurality of rAAV particles comprising a nucleic acid expression construct comprising a functional human TNNT2 coding sequence operably linked to a promoter and optionally an enhancer element, wherein the expression construct is flanked on each side by an inverted terminal repeat sequence, and wherein said contacting results in the target cell increasing expression of functional human TNNT2 as compared to prior to the contacting, thereby increasing the expression of functional human TNNT2.

Also described herein is a method of inducing increased expression of human TNNT2 in a target cell, comprising contacting a target cell with a plurality of rAAV particles, wherein the rAAV particles comprise a nucleic acid expression construct comprising a functional human TNNT2 coding sequence operably linked to a promoter and optionally an enhancer element, wherein the expression construct is flanked on each side by an inverted terminal repeat sequence, and wherein said contacting results in the target cell increasing expression of functional human TNNT2 as compared to prior to the contacting, thereby increasing the expression of functional human TNNT2.

In some embodiments, the contacting is in vivo. In some embodiments, the method is used for the treatment of dilated cardiomyopathy. In some embodiments, the method is used for the treatment of hypertrophic cardiomyopathy. In some embodiments, the nucleic acids, the rAAV particles, the compositions, or the methods of manufacture described herein can be used for the treatment of dilated cardiomyopathy or hypertrophic cardiomyopathy.

### BRIEF DESCRIPTION OF THE DRAWINGS

FIG. 1 shows a non-limiting example of a gene construct map for a construct embodiment disclosed herein.

FIG. 2 shows a non-limiting example of a gene construct map for a construct embodiment disclosed herein.

FIG. 3 shows a non-limiting example of a gene construct map for a construct embodiment disclosed herein.

FIG. 4 shows a non-limiting example of a gene construct map for a construct embodiment disclosed herein.

FIG. 5 shows a non-limiting example of a gene construct map for a construct embodiment disclosed herein.

FIG. 6 shows a Western blot of CMV-TNNT2 HEK293 lysates.

FIG. 7A shows a Western blot of Des1-TNNT2 C2C12 lysates.

FIG. 7B shows Des1-TNNT2 constructs with and without specific features.

FIG. 7C shows qPCR results for TNNT2 constructs.

FIG. 8 shows mean body weight after in vivo dosing with constructs provided for herein.

FIG. 9 shows mean heart weights after in vivo dosing with constructs provided for herein.

FIG. 10 shows Heart Vector Copy Number after dosing with constructs provided for herein.

FIG. 11 shows Liver Vector Copy Number after dosing with constructs provided for herein.

FIG. 12 shows qPCR results of heart tissue after dosing with constructs provided for herein.

FIG. 13 shows qPCR results of liver tissue after dosing with constructs provided for herein.

### DETAILED DESCRIPTION

Reference is made to particular features and/or non-limiting embodiments of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

A “subject” refers to mammal that is the object of treatment using a method or composition as provided for herein. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. To “treat” a disease as the term is used herein, means to reduce the frequency or severity of at least one sign or symptom of a disease or disorder experienced by a subject.

The term “effective amount,” as used herein, refers to an amount that is capable of treating or ameliorating a disease or condition or otherwise capable of producing an intended therapeutic effect, such as reducing the frequency or severity of at least one sign or symptom of a disease or disorder experienced by a subject.

A “nucleic acid” sequence refers to a deoxyribonucleic acid (DNA) or ribonucleic acid (RNA) sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “polynucleotide,” refers to a polymeric form of nucleotides of any length, including DNA, RNA, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

The term “isolated” when referring to a nucleotide sequence, means that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not materially affect the basic characteristics of the composition.

The term “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100.

For the purpose of describing the relative position of nucleotide sequences in a particular nucleic acid molecule throughout the instant application, such as when a particular nucleotide sequence is described as being situated “upstream,” “downstream,” “3′,” or “5” relative to another sequence, it is to be understood that it is the position of the sequences in the “sense” or “coding” strand of a DNA molecule that is being referred to as is conventional in the art.

Sequence identity can be determined by aligning sequences using algorithms, such as BESTFIT, FASTA, and TFASTA in the Wisconsin Genetics Software Package Release 7.0, (Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “recombinant,” as applied to a polynucleotide means that the polynucleotide is the product of various combinations of cloning, restriction or ligation steps, and other procedures that result in a construct that is distinct from a polynucleotide found in nature and/or a combination of polynucleotides and viral proteins that is not found in nature. A recombinant virus is a viral particle comprising a recombinant polynucleotide. The terms respectively include replicates of the original polynucleotide construct and progeny of the original virus construct.

The term “gene,” refers to a polynucleotide containing at least one open reading frame that is capable of encoding a particular gene product. Any of the polynucleotide sequences described herein may be used to identify larger fragments or full-length coding sequences of the genes with which they are associated. Methods of isolating larger fragment sequences are known to those of skill in the art.

The term “transgene,” as used herein, refers to a nucleic acid sequence to be positioned within a viral vector and encoding a polypeptide, protein or other product of interest. In some embodiments, one rAAV vector may comprise a sequence encoding one or more transgenes (which can optionally be the same gene, or different genes). For example, one rAAV vector may comprise the coding sequence for 1, 2, 3, 4, 5, 6, 7, 8, 9, or 10 transgenes. The transgenes of the present disclosure relate to the improvement of one or more heart conditions, such as cardiomyopathies as provided for herein.

The terms “gene transfer” or “gene delivery” refer to methods or systems for inserting DNA, such as a transgene, into host cells, such as those of a subject afflicted with a cardiomyopathy. In several embodiments, gene transfer yields transient expression of non-integrated transferred DNA, extrachromosomal replication and expression of transferred replicons (e.g., episomes). In additional embodiments, gene transfer results in integration of transferred genetic material into the genomic DNA of host cells.

The terms “regulatory element” or “regulatory sequence”, or variations thereof, refer to a nucleotide sequence that participates in functional regulation of a polynucleotide, including replication, duplication, transcription, splicing, translation, or degradation of the polynucleotide. Regulatory elements can be enhancing or inhibitory in nature, depending on the embodiment. Non-limiting examples of regulatory elements include transcriptional regulatory sequences such as promoter sequences, polyadenylation signals, transcription termination sequences, upstream regulatory domains, origins of replication, internal ribosome entry sites (“IRES”), enhancers, and the like. These elements collectively provide for the replication, transcription and translation of a coding sequence in a recipient cell, though not all of these sequences need always be present. It shall be appreciated that the structural components of a rAAV vector as provided for herein may be listed in individual paragraphs solely for clarity and may be used together in combination. For example, any regulatory element or other component can be used in combination with any transgene (or transgenes) provided for herein.

A “promoter” is a polynucleotide that interacts with an RNA polymerase and initiates transcription of a coding region (e.g., a transgene) usually located downstream (in the 3′ direction) from the promoter.

The term “operably linked” refers to an arrangement of elements wherein the components are configured to perform a function. For example, regulatory sequences operably linked to a coding sequence result in the expression of the coding sequence. Depending on the embodiment, a regulatory sequence need not be contiguous with the coding sequence. Thus, for example, one or more untranslated, yet transcribed, sequences can be present between a promoter sequence and a coding sequence, with those two sequences still being considered “operably linked”.

The term “vector” means any molecular vehicle, such as a plasmid, phage, transposon, cosmid, chromosome, virus, viral particle, virion, etc. which can transfer gene sequences (e.g., a transgene) to or between cells of interest.

An “expression vector” is a vector comprising a region of nucleic acid (e.g., a transgene) which encodes a gene product (e.g., a polypeptide or protein) of interest. As disclosed herein, vectors are used for achieving expression, e.g., stable expression, of a protein in an intended target cell. An expression vector may also comprise control elements operatively linked to the transgene to facilitate expression of the encoded protein in the target cell. A combination of one or more regulatory elements and a gene or genes to which they are operably linked for expression may be referred to herein as an “expression cassette.”

The term “AAV” is an abbreviation for adeno-associated virus, and may be used to refer to the virus itself or derivatives thereof. The term covers all subtypes and both naturally occurring and recombinant forms, unless otherwise indicated. The abbreviation “rAAV” refers to recombinant adeno-associated virus, also referred to as a recombinant AAV vector (or “rAAV vector”), which refers to AAV comprising a polynucleotide sequence not of AAV origin (e.g., a transgene). The term “AAV” includes AAV serotype 1 (AAV-1), AAV serotype 2 (AAV-2), AAV serotype 3 (AAV-3), AAV serotype 4 (AAV-4), AAV serotype 5 (AAV-5), AAV serotype 6 (AAV-6), AAV serotype 7 (AAV-7), AAV serotype 8 (AAV-8), AAV serotype 9 (AAV-9), serotype rh10 AAV, serotype rh74 AAV, or a pseudotyped rAAV (e.g., AAV2/9, referring an AAV vector with the genome of AAV2 (e.g., the ITRs of AAV2) and the capsid of AAV9). In several embodiments, the preferred serotype for delivery to human patients affected by a cardiomyopathy is one of AAV-9, serotype rh74, serotype rh10, or AAV-8. In several embodiments, an rh74 AAV is mutated to advantageously enhance delivery to cardiac tissue, for example by a tryptophan to arginine mutation at amino acid 505 of VP1 capsid, or other mutations, as described in PCT Publication WO 2019/178412, which is incorporated in its entirety by reference herein.

The term “AAV virus” or “AAV viral particle” or “rAAV vector particle” refers to a viral particle composed of at least AAV capsid protein and an encapsidated polynucleotide.

The term “heterologous” refers to genotypically distinct origins. For example, a heterologous polynucleotide is one derived from a different species as compared to a reference species (for example a human gene inserted into a viral plasmid is a heterologous gene). A promoter removed from its native coding sequence and operatively linked to a coding sequence with which it is not naturally found linked is a heterologous promoter.

As used herein, the term “kit” may be used to describe variations of the portable, self-contained enclosure that includes at least one set of components to conduct one or more of the diagnostic or therapeutic methods of the present disclosure.

The term “carrier” refers to a diluent, adjuvant, excipient, or vehicle with which the rAAV particle or preparation, and/or rAAV vectors is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions may also be employed as liquid carriers.

As used herein, “Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allele-specific” gene silencing refers to the specific silencing of one allele of a gene.

As used herein, “Knock-down,” “knock-down technology” refers to a technique of gene silencing in which the expression of a target gene is reduced as compared to the gene expression prior to the introduction of the RNAi molecule, which can lead to the inhibition of production of the target gene product. The term “reduced” is used herein to indicate that the target gene expression is lowered by 1-100%. For example, the expression may be reduced by 10, 20, 30, 40, 50, 60, 70, 80, 90, 95, or even 99%. Knock-down of gene expression can be directed by the use of dsRNAs or siRNAs. For example, “RNA interference (RNAi),” which can involve the use of siRNA, has been successfully applied to knockdown the expression of specific genes in plants, D. melanogaster, C. elegans, trypanosomes, planaria, hydra, and several vertebrate species including the mouse.

As used herein, “RNA interference (RNAi)” is the process of sequence-specific, post-transcriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, RNAi molecules induce degradation of target mRNA with consequent sequence-specific inhibition of gene expression.

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. The “sense” and “antisense” sequences can be used with or without a loop region to form siRNA molecules. As used herein, the term siRNA is meant to be equivalent to other terms used to describe nucleic acid molecules that are capable of mediating sequence specific RNAi, for example, double-stranded RNA (dsRNA), micro-RNA (miRNA), short hairpin RNA (shRNA), short interfering oligonucleotide, short interfering nucleic acid, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetic silencing. For example, siRNA molecules of the invention can be used to epigenetically silence genes at both the post-transcriptional level or the pre-transcriptional level. In a non-limiting example, epigenetic modulation of gene expression by siRNA molecules of the invention can result from siRNA mediated modification of chromatin structure or methylation pattern to alter gene expression. In another non-limiting example, modulation of gene expression by siRNA molecules of the invention can result from siRNA mediated cleavage of RNA (either coding or non-coding RNA) via RISC, or alternately, translational inhibition as is known in the art.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous or identical includes 96%, 97%, 98%, 99%, and 100% homologous or identical to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

### SEQUENCE LISTING

Construct 1 (“pdsTR2-TNNT2-
intron-mini-TNNT2”)

| SEQ | Elements | |
|---|---|---|
| ID: | (5′ -> 3′) | NT sequence |
| 1 | ITR-L | TTGGCCACTCCCTCTCTGCGCGCTC |
| | (“ITRdeltaL”) | GCTCGCTCACTGAGGCCGGGCGACC |
| | | AAAGGTCGCCCGACGCCCGGGCTTT |
| | | GCCCGGGCGGCCTCAGTGAGCGAGC |
| | | GAGCGCGCAGAGAGGGAGTGGCCA |
| 2 | Spacer | TCTAGAGGCGCGCCAAGCTT |
| 3 | TNNT2 | GTCATGGAGAAGACCCACCTTGCAG |
| | promoter | ATGTCCTCACTGGGGCTGGCAGAGC |
| | | CGGCAACCTGCCTAAGGCTGCTCAG |
| | | TCCATTAGGAGCCAGTAGCCTGGAA |
| | | GATGTCTTTACCCCCAGCATCAGTT |
| | | CAAGTGGAGCAGCACATAACTCTTG |
| | | CCCTCTGCCTTCCAAGATTCTGGTG |
| | | CTGAGACTTATGGAGTGTCTTGGAG |
| | | GTTGCCTTCTGCCCCCCAACCCTGC |
| | | TCCCAGCTGGCCCTCCCAGGCCTGG |
| | | GTTGCTGGCCTCTGCTTTATCAGGA |
| | | TTCTCAAGAGGGACAGCTGGTTTAT |
| | | GTTGCATGACTGTTCCCTGCATATC |
| | | TGCTCTGGTTTTAAATAGCTTATCT |
| | | GAGCAGCTGGAGGACCACATGGGCT |
| | | TATATGGCGTGGGGTACATGATCCT |
| | | GTAGCCTTGTCCCTGGCACCTGCCA |
| | | AAATAGCAGCCAACACCCCCCACCC |
| | | CCACCGCCATCCCCCTGCCCCACCC |
| | | GTCCCCTGTCGCACATTCCTCCCTC |
| | | CGCAGGGCTGGCTCACCAGGCCCCA |
| | | GCCCACATGCCTGCTTAAAGCCCTC |
| | | TCCATCCTCTGCCTCACCCAGTCCC |
| | | CGCTGAGACTGAGCAGACGCCTCCA |
| 4 | Spacer | AGATCTGAATTC |
| 5 | Chimeric | CAGGTAAGTATCAAGGTTACAAGAC |
| | intron | AGGTTTAAGGAGACCAATAGAAACT |
| | | GGGCTTGTCGAGACAGAGGGCCGGC |
| | | CAAGACTCTTGCGTTTCTGATAGGC |
| | | ACCTATTGGTCTTACTGACATCCAC |
| | | TTTGCCTTTCTCTCCACAGGGT |
| 6 | Spacer | TCTAGAGGTACC |
| 7 | Exon 2 | GGAGAGCAGAGACC |
| | non-coding | |
| 8 | Exon 2 | ATGTCTGACATAGAAGAGGTGGTGG |
| | | AAGAGTACGAGGAGGAG |
| 9 | TNNT2 | GTGAGTATCTGGAGCATCTTGCCTG |
| | intron 1 | AGTGGGGTCCTCTCCCGCCGCTGCC |
| | with splice | CTGACACCTGGTCCAGGAGCCTCCC |
| | donor | AGCTGTCCCTCGGATTCTGGGTAGA |
| | | AGTAGCTGTGTGTGTTTTGGGCACC |
| | | CCGAGGAGAGATATAGCCATTTCCT |
| | | TTCCACTCCTGTACCCCTGGGTTGT |
| | | AGATCCCAGGCAGAGCACAGGCCGA |
| | | GAGACTTCGGGAAGCACAGAGGTTC |
| | | AGCTCCAGGGTCATCAGTGTCCTTG |
| | | GCCAGTCTTGGCTAG |
| 10 | FseI site | GGCCGGCC |
| 11 | TNNT2 | AAGTGAGCAGAACGCTGGTGAGACC |
| | intron 1 | AAGCTGGCTGGGGCGGAGGCTTGCG |
| | with splice | GAGGATGGTAGGAGGCAGCCCTGCA |
| | acceptor | TGAGAACGGCAGGCCAGGCTAGTGG |
| | | GTGTCATTGCAAGGTGGGCAGGGCA |
| | | GCGTGGACTCCACTAGGCAACAAGG |
| | | GAAAAGAAAGGGGGATTATCTTTGG |
| | | GGAAAGGCCAGTGTGTGCATGTGTG |
| | | TGCAGGCGTGTGTGTTTGCATGTGC |
| | | TTGTGTGCGAGCTACTGACAGTGTT |
| | | TCCTGTTGCTCTCAG |
| 12 | Exon 3 | GAGCAGGAA |
| 13 | Exon 4 | GAAGCAGCTGTTGAA |
| 14 | Exon 5 | GAAGAGGAGGACTGGAGAGAGGACG |
| | | AAGAC |
| 15 | Exon 6 | GAGCAGGAGGAGGCAGCGGAAGAGG |
| | | ATGCTGAAGCAGAGGCTGAGACCGA |
| | | GGAGACCAGGGCAGAA |
| 16 | Exon 7 | GAAGATGAAGAAGAAGAGGAAGCAA |
| | | AGGAGGCTGAA |
| 17 | Exon 8 | GATGGCCCAATGGAGGAGTCCAAAC |
| | | CAAAGCCCAGG |
| 18 | Exon 9 | TCGTTCATGCCCAACTTGGTGCCTC |
| | | CCAAGATCCCCGATGGAGAGAGAGT |
| | | GGACTTTGAT |
| 19 | Exon 10 | GACATCCACCGGAAGCGCATGGAGA |
| | | AGGACCTGAATGAGTTGCAGGCGCT |
| | | GATCGAGGCTCACTTTGAGAACAGG |
| | | AAGAAAGAGGAGGAGGAGCTCGTTT |
| | | CTCTCAAAGACAGGATCGAG |
| 20 | Exon 11 | AGACGTCGGGCAGAGCGGGCCGAGC |
| | | AGCAGCGCATCCGGAATGAGCGGGA |
| | | GAAGGAGCGGCAGAACCGCCTGGCT |
| 21 | Exon 12 | GAAGAGAGGGCTCGACGAGAGGAGG |
| | | AGGAGAACAGGAGGAAGGCTGAGGA |
| | | TGAGGCCCGGAAGAAGAAGGCTTTG |
| | | TCCAACATGATGCATTTTGGGGGTT |
| | | ACATCCAGAAG |
| 22 | Exon 13 | ACAGAGCGGAAAAGTGGGAAGAGGC |
| | | AGACTGAGCGGGAAAAGAAGAAGAA |
| | | GATTCTGGCTGAGAGGAGGAAGGTG |
| | | CTGGCCATTGACCACCTGAATGAAG |
| | | ATCAGCTGAGG |
| 23 | Exon 14 | GAGAAGGCCAAGGAGCTGTGGCAGA |
| | | GCATCTATAACTTGGAGGCAGAGAA |
| | | GTTCGACCTGCAGGAGAAGTTCAAG |
| | | CAGCAGAAATATGAG |
| 24 | Exon 15 | ATCAATGTTCTCCGAAACAGGATCA |
| | | ACGATAACCAGAAAGTC |
| 25 | Exon 16 | TCCAAGACCCGCGGGAAGGCTAAAG |
| | | TCACCGGGCGCTGGAAA |
| 26 | Stop | TAGTAATGA |
| | cassette | |
| 27 | TNNT2 | AGCCTGGCCTCCTTCACCAAAGATC |
| | 3′ UTR | TGCTCCTCGCTCGCACCTGCCTCCG |
| | | GCCTGCACTCCCCCAGTTCCCGGGC |
| | | CCTCCTGGGCACCCCAGGCAGCTCC |
| | | TGTTTGGAAATGGGGAGCTGGCCTA |
| | | GGTGGGAGCCACCACTCCTGCCTGC |
| | | CCCCACACCCACTCCACACCAGTAA |
| | | TAAAAAGCCACCACACACTGA |
| 28 | Spacer | GCGGCCGCCTCGAGGAGCTCCGGCC |
| | | G |
| 29 | bGH poly(A) | CTGTGCCTTCTAGTTGCCAGCCATC |
| | signal | TGTTGTTTGCCCCTCCCCCGTGCCT |
| | | TCCTTGACCCTGGAAGGTGCCACTC |
| | | CCACTGTCCTTTCCTAATAAAATGA |
| | | GGAAATTGCATC |
| 30 | Spacer | GCATGTCGACGCGCCGGCGTCTAGA |
| 31 | ITR-R | AGGAACCCCTAGTGATGGAGTTGGC |
| | | CACTCCCTCTCTGCGCGCTCGCTCG |
| | | CTCACTGAGGCCGGGCGACCAAAGG |
| | | TCGCCCGACGCCCGGGCTTTGCCCG |
| | | GGCGGCCTCAGTGAGCGAGCGAGCG |
| | | CGCAGAGAGGGAGTGGCCAA |

Construct 1 and 2 Protein Sequences

| | | AA sequence |
|---|---|---|
| SEQ | Elements | (N-term. -> C-term.) |
| ID: | (5′ -> 3′) | (as applicable) |
| 32 | Exon 2 | MSDIEEVVEEYEEE |
| 33 | Exon 3 | EQE |
| 34 | Exon 4 | EAAVE |
| 35 | Exon 5 | EEEDWREDED |
| 36 | Exon 6 | EQEEAAEEDAEAEAETEETRAE |
| 37 | Exon 7 | EDEEEEEAKEAE |
| 38 | Exon 8 | DGPMEESKPKPR |
| 39 | Exon 9 | SFMPNLVPPKIPDGERVDFD |
| 40 | Exon 10 | DIHRKRMEKDLNELQALIEAHFENR |
| | | KKEEEELVSLKDRIE |
| 41 | Exon 11 | RRRAERAEQQRIRNEREKERQNRLA |
| 42 | Exon 12 | EERARREEEENRRKAEDEARKKKAL |
| | | SNMMHFGGYIQK |
| 43 | Exon 13 | TERKSGKRQTEREKKKKILAERRKV |
| | | LAIDHLNEDQLR |
| 44 | Exon 14 | EKAKELWQSIYNLEAEKFDLQEKFK |
| | | QQKYE |
| 45 | Exon 15 | INVLRNRINDNQKV |
| 46 | Exon 16 | SKTRGKAKVTGRWK |
| SEQ | Elements | |
| ID: | (5′ -> 3′) | NT sequence |

Construct 2 (“pdsTR2-TNNT2-intron-
mini-TNNT2” without FseI site)

| | | |
|---|---|---|
| 47 | ITR-L | TTGGCCACTCCCTCTCTGCGCGCTC |
| | (“ITRdeltaL”) | GCTCGCTCACTGAGGCCGGGCGACC |
| | | AAAGGTCGCCCGACGCCCGGGCTTT |
| | | GCCCGGGCGGCCTCAGTGAGCGAGC |
| | | GAGCGCGCAGAGAGGGAGTGGCCA |
| 48 | Spacer | TCTAGAGGCGCGCCAAGCTT |
| 49 | TNNT2 | GTCATGGAGAAGACCCACCTTGCAG |
| | promoter | ATGTCCTCACTGGGGCTGGCAGAGC |
| | | CGGCAACCTGCCTAAGGCTGCTCAG |
| | | TCCATTAGGAGCCAGTAGCCTGGAA |
| | | GATGTCTTTACCCCCAGCATCAGTT |
| | | CAAGTGGAGCAGCACATAACTCTTG |
| | | CCCTCTGCCTTCCAAGATTCTGGTG |
| | | CTGAGACTTATGGAGTGTCTTGGAG |
| | | GTTGCCTTCTGCCCCCCAACCCTGC |
| | | TCCCAGCTGGCCCTCCCAGGCCTGG |
| | | GTTGCTGGCCTCTGCTTTATCAGGA |
| | | TTCTCAAGAGGGACAGCTGGTTTAT |
| | | GTTGCATGACTGTTCCCTGCATATC |
| | | TGCTCTGGTTTTAAATAGCTTATCT |
| | | GAGCAGCTGGAGGACCACATGGGCT |
| | | TATATGGCGTGGGGTACATGATCCT |
| | | GTAGCCTTGTCCCTGGCACCTGCCA |
| | | AAATAGCAGCCAACACCCCCCACCC |
| | | CCACCGCCATCCCCCTGCCCCACCC |
| | | GTCCCCTGTCGCACATTCCTCCCTC |
| | | CGCAGGGCTGGCTCACCAGGCCCCA |
| | | GCCCACATGCCTGCTTAAAGCCCTC |
| | | TCCATCCTCTGCCTCACCCAGTCCC |
| | | CGCTGAGACTGAGCAGACGCCTCCA |
| 50 | Spacer | AGATCTGAATTC |
| 51 | Chimeric | CAGGTAAGTATCAAGGTTACAAGAC |
| | intron | AGGTTTAAGGAGACCAATAGAAACT |
| | | GGGCTTGTCGAGACAGAGGGCCGGC |
| | | CAAGACTCTTGCGTTTCTGATAGGC |
| | | ACCTATTGGTCTTACTGACATCCAC |
| | | TTTGCCTTTCTCTCCACAGGGT |
| 52 | Spacer | TCTAGAGGTACC |
| 53 | Exon 2 | GGAGAGCAGAGACC |
| | non-coding | |
| 54 | Exon 2 | ATGTCTGACATAGAAGAGGTGGTGG |
| | | AAGAGTACGAGGAGGAG |
| 55 | TNNT2 | GTGAGTATCTGGAGCATCTTGCCTG |
| | intron 1 | AGTGGGGTCCTCTCCCGCCGCTGCC |
| | | CTGACACCTGGTCCAGGAGCCTCCC |
| | | AGCTGTCCCTCGGATTCTGGGTAGA |
| | | AGTAGCTGTGTGTGTTTTGGGCACC |
| | | CCGAGGAGAGATATAGCCATTTCCT |
| | | TTCCACTCCTGTACCCCTGGGTTGT |
| | | AGATCCCAGGCAGAGCACAGGCCGA |
| | | GAGACTTCGGGAAGCACAGAGGTTC |
| | | AGCTCCAGGGTCATCAGTGTCCTTG |
| | | GCCAGTCTTGGCTAGAAGTGAGCAG |
| | | AACGCTGGTGAGACCAAGCTGGCTG |
| | | GGGCGGAGGCTTGCGGAGGATGGTA |
| | | GGAGGCAGCCCTGCATGAGAACGGC |
| | | AGGCCAGGCTAGTGGGTGTCATTGC |
| | | AAGGTGGGCAGGGCAGCGTGGACTC |
| | | CACTAGGCAACAAGGGAAAAGAAAG |
| | | GGGGATTATCTTTGGGGAAAGGCCA |
| | | GTGTGTGCATGTGTGTGCAGGCGTG |
| | | TGTGTTTGCATGTGCTTGTGTGCGA |
| | | GCTACTGACAGTGTTTCCTGTTGCT |
| | | CTCAG |
| 56 | Exon 3 | GAGCAGGAA |
| 57 | Exon 4 | GAAGCAGCTGTTGAA |
| 58 | Exon 5 | GAAGAGGAGGACTGGAGAGAGGACG |
| | | AAGAC |
| 59 | Exon 6 | GAGCAGGAGGAGGCAGCGGAAGAGG |
| | | ATGCTGAAGCAGAGGCTGAGACCGA |
| | | GGAGACCAGGGCAGAA |
| 60 | Exon 7 | GAAGATGAAGAAGAAGAGGAAGCAA |
| | | AGGAGGCTGAA |
| 61 | Exon 8 | GATGGCCCAATGGAGGAGTCCAAAC |
| | | CAAAGCCCAGG |
| 62 | Exon 9 | TCGTTCATGCCCAACTTGGTGCCTC |
| | | CCAAGATCCCCGATGGAGAGAGAGT |
| | | GGACTTTGAT |
| 63 | Exon 10 | GACATCCACCGGAAGCGCATGGAGA |
| | | AGGACCTGAATGAGTTGCAGGCGCT |
| | | GATCGAGGCTCACTTTGAGAACAGG |
| | | AAGAAAGAGGAGGAGGAGCTCGTTT |
| | | CTCTCAAAGACAGGATCGAG |
| 64 | Exon 11 | AGACGTCGGGCAGAGCGGGCCGAGC |
| | | AGCAGCGCATCCGGAATGAGCGGGA |
| | | GAAGGAGCGGCAGAACCGCCTGGCT |
| 65 | Exon 12 | GAAGAGAGGGCTCGACGAGAGGAGG |
| | | AGGAGAACAGGAGGAAGGCTGAGGA |
| | | TGAGGCCCGGAAGAAGAAGGCTTTG |
| | | TCCAACATGATGCATTTTGGGGGTT |
| | | ACATCCAGAAG |
| 66 | Exon 13 | ACAGAGCGGAAAAGTGGGAAGAGGC |
| | | AGACTGAGCGGGAAAAGAAGAAGAA |
| | | GATTCTGGCTGAGAGGAGGAAGGTG |
| | | CTGGCCATTGACCACCTGAATGAAG |
| | | ATCAGCTGAGG |
| 67 | Exon 14 | GAGAAGGCCAAGGAGCTGTGGCAGA |
| | | GCATCTATAACTTGGAGGCAGAGAA |
| | | GTTCGACCTGCAGGAGAAGTTCAAG |
| | | CAGCAGAAATATGAG |
| 68 | Exon 15 | ATCAATGTTCTCCGAAACAGGATCA |
| | | ACGATAACCAGAAAGTC |
| 69 | Exon 16 | TCCAAGACCCGCGGGAAGGCTAAAG |
| | | TCACCGGGCGCTGGAAA |
| 70 | Stop | TAGTAATGA |
| | cassette | |
| 71 | TNNT2 | AGCCTGGCCTCCTTCACCAAAGATC |
| | 3′ UTR | TGCTCCTCGCTCGCACCTGCCTCCG |
| | | GCCTGCACTCCCCCAGTTCCCGGGC |
| | | CCTCCTGGGCACCCCAGGCAGCTCC |
| | | TGTTTGGAAATGGGGAGCTGGCCTA |
| | | GGTGGGAGCCACCACTCCTGCCTGC |
| | | CCCCACACCCACTCCACACCAGTAA |
| | | TAAAAAGCCACCACACACTGA |
| 72 | Spacer | GCGGCCGCCTCGAGGAGCTCCGGCC |
| | | G |
| 73 | bGH poly(A) | CTGTGCCTTCTAGTTGCCAGCCATC |
| | signal | TGTTGTTTGCCCCTCCCCCGTGCCT |
| | | TCCTTGACCCTGGAAGGTGCCACTC |
| | | CCACTGTCCTTTCCTAATAAAATGA |
| | | GGAAATTGCATC |
| 74 | Spacer | GCATGTCGACGCGCCGGCGTCTAGA |
| 75 | ITR-R | AGGAACCCCTAGTGATGGAGTTGGC |
| | | CACTCCCTCTCTGCGCGCTCGCTCG |
| | | CTCACTGAGGCCGGGCGACCAAAGG |
| | | TCGCCCGACGCCCGGGCTTTGCCCG |
| | | GGCGGCCTCAGTGAGCGAGCGAGCG |
| | | CGCAGAGAGGGAGTGGCCAA |

Construct 3 (“pdsTR2-Des1-TNNT2 Dual Plan)

| | | |
|---|---|---|
| 76 | ITR-L | TTGGCCACTCCCTCTCTGCGCGCTC |
| | (“ITRdeltaL”) | GCTCGCTCACTGAGGCCGGGCGACC |
| | | AAAGGTCGCCCGACGCCCGGGCTTT |
| | | GCCCGGGCGGCCTCAGTGAGCGAGC |
| | | GAGCGCGCAGAGAGGGAGTGGCCA |
| 77 | Des 1 | TAGACTAGCATGCTGCCCATGTAAG |
| | | GAGGCAAGGCCTGGGGACACCCGAG |
| | | ATGCCTGGTTATAATTAACCCAGAC |
| | | ATGTGGCTGCCCCCCCCCCCCCAAC |
| | | ACCTGCTGCCTCTAAAAATAACCCT |
| | | GCATGCCATGTTCCCGGCGAAGGGC |
| | | CAGCTGTCCCCCGCCAGCTAGACTC |
| | | AGCACTTAGTTTAGGAACCAGTGAG |
| | | CAAGTCAGCCCTTGGGGCAGCCCAT |
| | | ACAAGGCCATGGGGCTGGGCAAGCT |
| | | GCACGCCTGGGTCCGGGGTGGGCAC |
| | | GGTGCCCGGGCAACGAGCTGAAAGC |
| | | TCATCTGCTCTCAGGGGCCCCTCCC |
| | | TGGGGACAGCCCCTCCTGGCTAGTC |
| | | ACACCCTGTAGGCTCCTCTATATAA |
| | | CCCAGGGGCACAGGGGCTGCCCTCA |
| | | TTCTACCACCACCTCCACAGCACAG |
| | | ACAGACACTCAGGAGCCAGCCAAA |
| 78 | Exon 2 | GGAGAGCAGAGACC |
| | Non-Coding | |
| 79 | Exon 2 | ATGTCTGACATAGAAGAGGTGGTGG |
| | | AAGAGTACGAGGAGGAG |
| 80 | Intron 1 sd | GTGAGTATCTGGAGCATCTTGCCTG |
| | | AGTGGGGTCCTCTCCCGCCGCTGCC |
| | | CTGACACCTGGTCCAGGAGCCTCCC |
| | | AGCTGTCCCTCGGATTCTGGGTAGA |
| | | AGTAGCTGTGTGTGTTTTGGGCACC |
| | | CCGAGGAGAGATATAGCCATTTCCT |
| | | TTCCACTCCTGTACCCCTGGGTTGT |
| | | AGATCCCAGGCAGAGCACAGGCCGA |
| | | GAGACTTCGGGAAGCACAGAGGTTC |
| | | AGCTCCAGGGTCATCAGTGTCCTTG |
| | | GCCAGTCTTGGCTAG |
| 81 | Intron 1 sa | AAGTGAGCAGAACGCTGGTGAGACC |
| | | AAGCTGGCTGGGGCGGAGGCTTGCG |
| | | GAGGATGGTAGGAGGCAGCCCTGCA |
| | | TGAGAACGGCAGGCCAGGCTAGTGG |
| | | GTGTCATTGCAAGGTGGGCAGGGCA |
| | | GCGTGGACTCCACTAGGCAACAAGG |
| | | GAAAAGAAAGGGGGATTATCTTTGG |
| | | GGAAAGGCCAGTGTGTGCATGTGTG |
| | | TGCAGGCGTGTGTGTTTGCATGTGC |
| | | TTGTGTGCGAGCTACTGACAGTGTT |
| | | TCCTGTTGCTCTCAG |
| 82 | Exon 3 | GAGCAGGAA |
| 83 | Exon 4 | GAAGCAGCTGTTGAA |
| 84 | Exon 5 | GAAGAGGAGGACTGGAGAGAGGACG |
| | | AAGAC |
| 85 | Exon 6 | GAGCAGGAGGAGGCAGCGGAAGAGG |
| | | ATGCTGAAGCAGAGGCTGAGACCGA |
| | | GGAGACCAGGGCAGAA |
| 86 | Exon 7 | GAAGATGAAGAAGAAGAGGAAGCAA |
| | | AGGAGGCTGAA |
| 87 | Exon 8 | GATGGCCCAATGGAGGAGTCCAAAC |
| | | CAAAGCCCAGG |
| 88 | Exon 9 | TCGTTCATGCCCAACTTGGTGCCTC |
| | | CCAAGATCCCCGATGGAGAGAGAGT |
| | | GGACTTTGAT |
| 89 | Exon 10 | GACATCCACCGGAAGCGCATGGAGA |
| | | AGGACCTGAATGAGTTGCAGGCGCT |
| | | GATCGAGGCTCACTTTGAGAACAGG |
| | | AAGAAAGAGGAGGAGGAGCTCGTTT |
| | | CTCTCAAAGACAGGATCGAG |
| 90 | Exon 11 | AGACGTCGGGCAGAGCGGGCCGAGC |
| | | AGCAGCGCATCCGGAATGAGCGGGA |
| | | GAAGGAGCGGCAGAACCGCCTGGCT |
| 91 | Exon 12 | GAAGAGAGGGCTCGACGAGAGGAGG |
| | | AGGAGAACAGGAGGAAGGCTGAGGA |
| | | TGAGGCCCGGAAGAAGAAGGCTTTG |
| | | TCCAACATGATGCATTTTGGGGGTT |
| | | ACATCCAGAAG |
| 92 | Exon 13 | ACAGAGCGGAAAAGTGGGAAGAGGC |
| | | AGACTGAGCGGGAAAAGAAGAAGAA |
| | | GATTCTGGCTGAGAGGAGGAAGGTG |
| | | CTGGCCATTGACCACCTGAATGAAG |
| | | ATCAGCTGAGG |
| 93 | Exon 14 | GAGAAGGCCAAGGAGCTGTGGCAGA |
| | | GCATCTATAACTTGGAGGCAGAGAA |
| | | GTTCGACCTGCAGGAGAAGTTCAAG |
| | | CAGCAGAAATATGAG |
| 94 | Exon 15 | ATCAATGTTCTCCGAAACAGGATCA |
| | | ACGATAACCAGAAAGTC |
| 95 | Exon 16 | TCCAAGACCCGCGGGAAGGCTAAAG |
| | | TCACCGGGCGCTGGAAATAG |
| 96 | 3′ UTR | AGCCTGGCCTCCTTCACCAAAGATC |
| | | TGCTCCTCGCTCGCACCTGCCTCCG |
| | | GCCTGCACTCCCCCAGTTCCCGGGC |
| | | CCTCCTGGGCACCCCAGGCAGCTCC |
| | | TGTTTGGAAATGGGGAGCTGGCCTA |
| | | GGTGGGAGCCACCACTCCTGCCTGC |
| | | CCCCACACCCACTCCACACCAGTAA |
| | | TAAAAAGCCACCACACACTGA |
| 97 | Poly A | AATAAAAGATCCTTATTTTCATTGG |
| | | ATCTGTGTGTTGGTTTTTTGTGTG |
| 98 | ITR-R | AGGAACCCCTAGTGATGGAGTTGGC |
| | | CACTCCCTCTCTGCGCGCTCGCTCG |
| | | CTCACTGAGGCCGGGCGACCAAAGG |
| | | TCGCCCGACGCCCGGGCTTTGCCCG |
| | | GGCGGCCTCAGTGAGCGAGCGAGCG |
| | | CGCAGAGAGGGAGTGGCCAA |

Construct 4 (“pTR2-MHCK9-TNNT2_Dual2_plan”)

| | | |
|---|---|---|
| 99 | ITR-L | TTGGCCACTCCCTCTCTGCGCGCTC |
| | (“ITRdeltaL”) | GCTCGCTCACTGAGGCCGGGCGACC |
| | | AAAGGTCGCCCGACGCCCGGGCTTT |
| | | GCCCGGGCGGCCTCAGTGAGCGAGC |
| | | GAGCGCGCAGAGAGGGAGTGGCCAA |
| | | CTCCATCACTAGGGGTTCCT |
| 100 | Alpha MHC | ACCCTTCAGATTAAAAATAACTGAG |
| | enhancer | GTAAGGGCCTGGGTAGGGGAGGTGG |
| | | TGTGAGACGCTCCTGTCTCTCCTCT |
| | | ATCTGCCCATCGGCCCTTTGGGGAG |
| | | GAGGAATGTGCCCAAGGACTAAAAA |
| | | AAGGCCATGGAGCCAGAGGGGCGAG |
| | | GGCAACAGACCTTTCATGGGCAAAC |
| | | CTTGGGGCCCTGCTGT |
| 101 | MHCK9 (MCK) | CTGCCCATGTAAGGAGGCAAGGCCT |
| | Enhancer | GGGGACACCCGAGATGCCTGGTTAT |
| | | AATTAACCCAGACATGTGGCTGCCC |
| | | CCCCCCCCCCAACACCTGCTGCCTC |
| | | TAAAAATAACC |
| 102 | MHCK9 (MCK) | GTTCCCGGCGAAGGGCCAGCTGTCC |
| | Promoter | CCCGCCAGCTAGACTCAGCACTTAG |
| | | TTTAGGAACCAGTGAGCAAGTCAGC |
| | | CCTTGGGGCAGCCCATACAAGGCCA |
| | | TGGGGCTGGGCAAGCTGCACGCCTG |
| | | GGTCCGGGGTGGGCACGGTGCCCGG |
| | | GCAACGAGCTGAAAGCTCATCTGCT |
| | | CTCAGGGGCCCCTCCCTGGGGACAG |
| | | CCCCTCCTGGCTAGTCACACCCTGT |
| | | AGGCTCCTCTATATAACCCAGGGGC |
| | | ACAGGGGCTGCCCTC |
| 103 | MHCK9 (MCK) | ACCACCACCTCCACAGCACAGACAG |
| | 5′ UTR | ACACTCAGGAGCAGCCAG |
| 104 | Exon 2 | GGAGAGCAGAGACC |
| | Non-coding | |
| 105 | Exon 2 | ATGTCTGACATAGAAGAGGTGGTGG |
| | | AAGAGTACGAGGAGGAG |
| 106 | Intron 1 sd | GTGAGTATCTGGAGCATCTTGCCTG |
| | | AGTGGGGTCCTCTCCCGCCGCTGCC |
| 107 | Intron 1 sa | TTGCATGTGCTTGTGTGCGAGCTAC |
| | | TGACAGTGTTTCCTGTTGCTCTCAG |
| 108 | Exon 3 | GAGCAGGAA |
| 109 | Exon 4 | GAAGCAGCTGTTGAA |
| 110 | Exon 5 | GAAGAGGAGGACTGGAGAGAGGACG |
| | | AAGAC |
| 111 | Exon 6 | GAGCAGGAGGAGGCAGCGGAAGAGG |
| | | ATGCTGAAGCAGAGGCTGAGACCGA |
| | | GGAGACCAGGGCAGAA |
| 112 | Exon 7 | GAAGATGAAGAAGAAGAGGAAGCAA |
| | | AGGAGGCTGAA |
| 113 | Exon 8 | GATGGCCCAATGGAGGAGTCCAAAC |
| | | CAAAGCCCAGG |
| 114 | Exon 9 | TCGTTCATGCCCAACTTGGTGCCTC |
| | | CCAAGATCCCCGATGGAGAGAGAGT |
| | | GGACTTTGAT |
| 115 | Exon 10 | GACATCCACCGGAAGCGCATGGAGA |
| | | AGGACCTGAATGAGTTGCAGGCGCT |
| | | GATCGAGGCTCACTTTGAGAACAGG |
| | | AAGAAAGAGGAGGAGGAGCTCGTTT |
| | | CTCTCAAAGACAGGATCGAG |
| 116 | Exon 11 | AGACGTCGGGCAGAGCGGGCCGAGC |
| | | AGCAGCGCATCCGGAATGAGCGGGA |
| | | GAAGGAGCGGCAGAACCGCCTGGCT |
| 117 | Exon 12 | GAAGAGAGGGCTCGACGAGAGGAGG |
| | | AGGAGAACAGGAGGAAGGCTGAGGA |
| | | TGAGGCCCGGAAGAAGAAGGCTTTG |
| | | TCCAACATGATGCATTTTGGGGGTT |
| | | ACATCCAGAAG |
| 118 | Exon 13 | ACAGAGCGGAAAAGTGGGAAGAGGC |
| | | AGACTGAGCGGGAAAAGAAGAAGAA |
| | | GATTCTGGCTGAGAGGAGGAAGGTG |
| | | CTGGCCATTGACCACCTGAATGAAG |
| | | ATCAGCTGAGG |
| 119 | Exon 14 | GAGAAGGCCAAGGAGCTGTGGCAGA |
| | | GCATCTATAACTTGGAGGCAGAGAA |
| | | GTTCGACCTGCAGGAGAAGTTCAAG |
| | | CAGCAGAAATATGAG |
| 120 | Exon 15 | ATCAATGTTCTCCGAAACAGGATCA |
| | | ACGATAACCAGAAAGTC |
| 121 | Exon 16 | TCCAAGACCCGCGGGAAGGCTAAAG |
| | | TCACCGGGCGCTGGAAATAG |
| 122 | 3′ UTR | AGCCTGGCCTCCTTCACCAAAGATC |
| | | TGCTCCTCGCTCGCACCTGCCTCCG |
| | | GCCTGCACTCCCCCAGTTCCCGGGC |
| | | CCTCCTGGGCACCCCAGGCAGCTCC |
| | | TGTTTGGAAATGGGGAGCTGGCCTA |
| | | GGTGGGAGCCACCACTCCTGCCTGC |
| | | CCCCACACCCACTCCACACCAGTAA |
| | | TAAAAAGCCACCACACACTGA |
| 123 | BGH Poly A | CTGTGCCTTCTAGTTGCCAGCCATC |
| | | TGTTGTTTGCCCCTCCCCCGTGCCT |
| | | TCCTTGACCCTGGAAGGTGCCACTC |
| | | CCACTGTCCTTTCCTAATAAAATGA |
| | | GGAAATTGCATCGCATTGTCTGAGT |
| | | AGGTGTCATTCTATTCTGGGGGGTG |
| | | GGGTGGGGCAGGACAGCAAGGGGGA |
| | | GGATTGGGAAGACAATAGCAGGCAT |
| | | GCTGGGGA |
| 124 | ITR-R | AGGAACCCCTAGTGATGGAGTTGGC |
| | | CACTCCCTCTCTGCGCGCTCGCTCG |
| | | CTCACTGAGGCCGGGCGACCAAAGG |
| | | TCGCCCGACGCCCGGGCTTTGCCCG |
| | | GGCGGCCTCAGTGAGCGAGCGAGCG |
| | | CGCAGAGAGGGAGTGGCCAA |

Construct 5 (“dsTR2-Des1-TNNT2-pA-Dual”
AKA “#3 Kpnl-Des1-TNNT2-pA”)

| | | |
|---|---|---|
| 125 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 126 | Buffer | Cctagttgttaatagtaatcaatta |
| | Sequence | cggggtcattagttcatagcccata |
| | Between ITR | tatggagttccg |
| | and Des 1 | |
| 127 | Des 1-promoter | CCCTGCCCCCACAGCTCCTCTCCTG |
| | | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGGGGAGC |
| | | GCACATAGCAATTGGAAACTGAAAG |
| | | CTAATCAGACCCTTTCTGGAAATCA |
| | | GCCCACTGTTTATATACTTGAGGCC |
| | | CCACCCTCGAGATAACCAGGGCTGA |
| | | AAGAGGCCCGCCTGGGGGCTGCAGA |
| | | CATGCTTGCTGCCTGCCCTGGCGAA |
| | | GGATTGGCAGGCTTGCCCGTCACAG |
| | | GACCCCCGCTGGCTGACTCAGGGGC |
| | | GCAGGCCTCTTGCGGGGGAGCTGGC |
| | | CTCCCCGCCCCCACGGCCACGGGCC |
| | | GCCCTTTCCTGGCAGGACAGCGGGA |
| | | TCTTGCAGCTGTCAGGGGAGGGGAG |
| | | GCGGGGGCTGATGTCAGGAGGGATA |
| | | CAAATAGTGCCGACGGCTGGGGGCC |
| | | CTGTCTCCCCTCGCCGCATCCACTC |
| | | TCCGGCCGGccgcctgcccgccgcc |
| | | tcctccgtgcgcccgccagcctcgc |
| | | ccgcgccgtca |
| 128 | Kozak | GCCACC |
| 129 | TNNT2 | ATGtctgacatagaagaggtggtgg |
| | | aagagtacgaggaggaggagcagga |
| | | agaagcagctgttgaagaagaggag |
| | | gactggagagaggacgaagacgagc |
| | | aggaggaggcagcggaagaggatgc |
| | | tgaagcagaggctgagaccgaggag |
| | | accagggcagaagaagatgaagaag |
| | | aagaggaagcaaaggaggctgaaga |
| | | tggcccaatggaggagtccaaacca |
| | | aagcccaggtcgttcatgcccaact |
| | | tggtgcctcccaagatccccgatgg |
| | | agagagagtggactttgatgacatc |
| | | caccggaagcgcatggagaaggacc |
| | | tgaatgagttgcaggcgctgatcga |
| | | ggctcactttgagaacaggaagaaa |
| | | gaggaggaggagctcgtttctctca |
| | | aagacaggatcgagagacgtcgggc |
| | | agagcgggccgagcagcagcgcatc |
| | | cggaatgagcgggagaaggagcggc |
| | | agaaccgcctggctgaagagagggc |
| | | tcgacgagaggaggaggagaacagg |
| | | aggaaggctgaggatgaggcccgga |
| | | agaagaaggctttgtccaacatgat |
| | | gcattttgggggttacatccagaag |
| | | acagagcggaaaagtgggaagaggc |
| | | agactgagcgggaaaagaagaagaa |
| | | gattctggctgagaggaggaaggtg |
| | | ctggccattgaccacctgaatgaag |
| | | atcagctgagggagaaggccaagga |
| | | gctgtggcagagcatctataacttg |
| | | gaggcagagaagttcgacctgcagg |
| | | agaagttcaagcagcagaaatatga |
| | | gatcaatgttctccgaaacaggatc |
| | | aacgataaccagaaagtctccaaga |
| | | cccgcgggaaggctaaagtcaccgg |
| | | gcgctggaaatag |
| 130 | Buffer | Tagtaatctagagcggccgcctcga |
| | Sequence | ggagctccggccg |
| 131 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 132 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggggcctcagtgagcgagcgagcgc |
| | | gcagagagggagtggccaa |

Construct 6 (“dsTR2-Des1-TNNT2-3UTR-pA-Dual”
AKA “#2 Knpl-DES1-TNNT2-3UTR”)

| | | |
|---|---|---|
| 133 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 134 | Buffer | cctagttgttaatagtaatcaatta |
| | Sequence | cggggtcattagttcatagcccata |
| | Between ITR | tatggagttccg |
| | and Des 1 | |
| 135 | Des 1- | CCCTGCCCCCACAGCTCCTCTCCTG |
| | promoter | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 136 | Kozak | GCCACC |
| 137 | TNNT2 | ATGtctgacatagaagaggtggtgg |
| | | aagagtacgaggaggaggagcagga |
| | | agaagcagctgttgaagaagaggag |
| | | gactggagagaggacgaagacgagc |
| | | aggaggaggcagcggaagaggatgc |
| | | tgaagcagaggctgagaccgaggag |
| | | accagggcagaagaagatgaagaag |
| | | aagaggaagcaaaggaggctgaaga |
| | | tggcccaatggaggagtccaaacca |
| | | aagcccaggtcgttcatgcccaact |
| | | tggtgcctcccaagatccccgatgg |
| | | agagagagtggactttgatgacatc |
| | | caccggaagcgcatggagaaggacc |
| | | tgaatgagttgcaggcgctgatcga |
| | | ggctcactttgagaacaggaagaaa |
| | | gaggaggaggagctcgtttctctca |
| | | aagacaggatcgagagacgtcgggc |
| | | agagcgggccgagcagcagcgcatc |
| | | cggaatgagcgggagaaggagcggc |
| | | agaaccgcctggctgaagagagggc |
| | | tcgacgagaggaggaggagaacagg |
| | | aggaaggctgaggatgaggcccgga |
| | | agaagaaggctttgtccaacatgat |
| | | gcattttgggggttacatccagaag |
| | | acagagcggaaaagtgggaagaggc |
| | | agactgagcgggaaaagaagaagaa |
| | | gattctggctgagaggaggaaggtg |
| | | ctggccattgaccacctgaatgaag |
| | | atcagctgagggagaaggccaagga |
| | | gctgtggcagagcatctataacttg |
| | | gaggcagagaagttcgacctgcagg |
| | | agaagttcaagcagcagaaatatga |
| | | gatcaatgttctccgaaacaggatc |
| | | aacgataaccagaaagtctccaaga |
| | | cccgcgggaaggctaaagtcaccgg |
| | | gcgctggaaatag |
| 138 | 3′ UTR | agcctggcctccttcaccaaagatc |
| | | tgctcctcgctcgcacctgcctccg |
| | | gcctgcactcccccagttcccgggc |
| | | cctcctgggcaccccaggcagctcc |
| | | tgtttggaaatggggagctggccta |
| | | ggtgggagccaccactcctgcctgc |
| | | ccccacacccactccacaccagtaa |
| | | taaaaagccaccacacactga |
| 139 | Buffer | tagtaatctagagcggccgcctcga |
| | Sequence | ggagctccggccg |
| 140 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 141 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

Construct 7 (“#1 KpnI-Des1-HGHint1-
TNNT2-3UTR-pA”)

| | | |
|---|---|---|
| 142 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 143 | Buffer | Cctagttgttaatagtaatcaatta |
| | Sequence | cggggtcattagttcatagcccata |
| | Between ITR | tatggagttccg |
| | and Des 1 | |
| 144 | Des 1- | CCCTGCCCCCACAGCTCCTCTCCTG |
| | promoter | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 145 | HGH Intron 1 | Gtaagcgcccctaaaatccctttgg |
| | | cacaatgtctcctgaggggagaggc |
| | | agcgacctgtagatgggacgggggc |
| | | actaaccctcagggtttggggttct |
| | | gaatgtgagtatcgccatctaagcc |
| | | cagtatttggccaatctcagaaagc |
| | | tcctggctccctggaggatggagag |
| | | agaaaaacaaacagctcctggagca |
| | | gggagagtgttggcctcttgctctc |
| | | cggctccctctgttgccctctggtt |
| | | tctccccag |
| 146 | Kozak | GCCACC |
| 147 | TNNT2 | ATGtctgacatagaagaggtggtgg |
| | | aagagtacgaggaggaggagcagga |
| | | agaagcagctgttgaagaagaggag |
| | | gactggagagaggacgaagacgagc |
| | | aggaggaggcagcggaagaggatgc |
| | | tgaagcagaggctgagaccgaggag |
| | | accagggcagaagaagatgaagaag |
| | | aagaggaagcaaaggaggctgaaga |
| | | tggcccaatggaggagtccaaacca |
| | | aagcccaggtcgttcatgcccaact |
| | | tggtgcctcccaagatccccgatgg |
| | | agagagagtggactttgatgacatc |
| | | caccggaagcgcatggagaaggacc |
| | | tgaatgagttgcaggcgctgatcga |
| | | ggctcactttgagaacaggaagaaa |
| | | gaggaggaggagctcgtttctctca |
| | | aagacaggatcgagagacgtcgggc |
| | | agagcgggccgagcagcagcgcatc |
| | | cggaatgagcgggagaaggagcggc |
| | | agaaccgcctggctgaagagagggc |
| | | tcgacgagaggaggaggagaacagg |
| | | aggaaggctgaggatgaggcccgga |
| | | agaagaaggctttgtccaacatgat |
| | | gcattttgggggttacatccagaag |
| | | acagagcggaaaagtgggaagaggc |
| | | agactgagcgggaaaagaagaagaa |
| | | gattctggctgagaggaggaaggtg |
| | | ctggccattgaccacctgaatgaag |
| | | atcagctgagggagaaggccaagga |
| | | gctgtggcagagcatctataacttg |
| | | gaggcagagaagttcgacctgcagg |
| | | agaagttcaagcagcagaaatatga |
| | | gatcaatgttctccgaaacaggatc |
| | | aacgataaccagaaagtctccaaga |
| | | cccgcgggaaggctaaagtcaccgg |
| | | gcgctggaaatag |
| 148 | 3′ UTR | Agcctggcctccttcaccaaagatc |
| | | tgctcctcgctcgcacctgcctccg |
| | | gcctgcactcccccagttcccgggc |
| | | cctcctgggcaccccaggcagctcc |
| | | tgtttggaaatggggagctggccta |
| | | ggtgggagccaccactcctgcctgc |
| | | ccccacacccactccacaccagtaa |
| | | taaaaagccaccacacactga |
| 149 | Buffer | Tagtaatctagagcggccgcctcga |
| | Sequence | ggagctccggccg |
| 150 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 151 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

Construct 8 (“#4 KpnI-Des1-HGHint1-TNNT2-pA”)

| | | |
|---|---|---|
| 152 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 153 | Buffer | Cctagttgttaatagtaatcaatta |
| | Sequence | cggggtcattagttcatagcccata |
| | Between ITR | tatggagttccg |
| | and Des 1 | |
| 154 | Des 1- | CCCTGCCCCCACAGCTCCTCTCCTG |
| | promoter | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 155 | HGH Intron 1 | Gtaagcgcccctaaaatccctttgg |
| | | cacaatgtctcctgaggggagaggc |
| | | agcgacctgtagatgggacgggggc |
| | | actaaccctcagggtttggggttct |
| | | gaatgtgagtatcgccatctaagcc |
| | | cagtatttggccaatctcagaaagc |
| | | tcctggctccctggaggatggagag |
| | | agaaaaacaaacagctcctggagca |
| | | gggagagtgttggcctcttgctctc |
| | | cggctccctctgttgccctctggtt |
| | | tctccccag |
| 156 | Kozak | GCCACC |
| 157 | TNNT2 | ATGtctgacatagaagaggtggtgg |
| | | aagagtacgaggaggaggagcagga |
| | | agaagcagctgttgaagaagaggag |
| | | gactggagagaggacgaagacgagc |
| | | aggaggaggcagcggaagaggatgc |
| | | tgaagcagaggctgagaccgaggag |
| | | accagggcagaagaagatgaagaag |
| | | aagaggaagcaaaggaggctgaaga |
| | | tggcccaatggaggagtccaaacca |
| | | aagcccaggtcgttcatgcccaact |
| | | tggtgcctcccaagatccccgatgg |
| | | agagagagtggactttgatgacatc |
| | | caccggaagcgcatggagaaggacc |
| | | tgaatgagttgcaggcgctgatcga |
| | | ggctcactttgagaacaggaagaaa |
| | | gaggaggaggagctcgtttctctca |
| | | aagacaggatcgagagacgtcgggc |
| | | agagcgggccgagcagcagcgcatc |
| | | cggaatgagcgggagaaggagcggc |
| | | agaaccgcctggctgaagagagggc |
| | | tcgacgagaggaggaggagaacagg |
| | | aggaaggctgaggatgaggcccgga |
| | | agaagaaggctttgtccaacatgat |
| | | gcattttgggggttacatccagaag |
| | | acagagcggaaaagtgggaagaggc |
| | | agactgagcgggaaaagaagaagaa |
| | | gattctggctgagaggaggaaggtg |
| | | ctggccattgaccacctgaatgaag |
| | | atcagctgagggagaaggccaagga |
| | | gctgtggcagagcatctataacttg |
| | | gaggcagagaagttcgacctgcagg |
| | | agaagttcaagcagcagaaatatga |
| | | gatcaatgttctccgaaacaggatc |
| | | aacgataaccagaaagtctccaaga |
| | | cccgcgggaaggctaaagtcaccgg |
| | | gcgctggaaatag |
| 158 | Buffer | tagtaatctagagcggccgcctcga |
| | Sequence | ggagctccggccg |
| 159 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 160 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

Construct 9 (“#5 KpnI-rs-Des1-TNNT2-rs-pA”)

| | | |
|---|---|---|
| 161 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 162 | Random | TAGAACGTTTCTAAGTTTTTCCATT |
| | Sequence | CAGTTAAACCGACTAAACTTTTCTG |
| | | CTTGAAGTTTTAATGAATTGTTATT |
| | | AACACCCAGATATTGTTTTAGAAAA |
| | | ATTATTCAAAAGCATTCACAACCAA |
| | | CTTATCGGGACTAGCCAAATTGATC |
| | | ATCAAGTTTATATAATAAAATACTA |
| | | AAAATTTAGTCTTACATTATGGAAT |
| 163 | Buffer | Ctagttgttaatagtaatcaattac |
| | Sequence | ggggtcattagttcatagcccatat |
| | Between ITR | atggagttccg |
| | and Des 1 | |
| 164 | Des 1- | CCCTGCCCCCACAGCTCCTCTCCTG |
| | promoter | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 165 | Kozak | GCCACC |
| 166 | TNNT2 | ATGtctgacatagaagaggtggtgg |
| | | aagagtacgaggaggaggagcagga |
| | | agaagcagctgttgaagaagaggag |
| | | gactggagagaggacgaagacgagc |
| | | aggaggaggcagcggaagaggatgc |
| | | tgaagcagaggctgagaccgaggag |
| | | accagggcagaagaagatgaagaag |
| | | aagaggaagcaaaggaggctgaaga |
| | | tggcccaatggaggagtccaaacca |
| | | aagcccaggtcgttcatgcccaact |
| | | tggtgcctcccaagatccccgatgg |
| | | agagagagtggactttgatgacatc |
| | | caccggaagcgcatggagaaggacc |
| | | tgaatgagttgcaggcgctgatcga |
| | | ggctcactttgagaacaggaagaaa |
| | | gaggaggaggagctcgtttctctca |
| | | aagacaggatcgagagacgtcgggc |
| | | agagcgggccgagcagcagcgcatc |
| | | cggaatgagcgggagaaggagcggc |
| | | agaaccgcctggctgaagagagggc |
| | | tcgacgagaggaggaggagaacagg |
| | | aggaaggctgaggatgaggcccgga |
| | | agaagaaggctttgtccaacatgat |
| | | gcattttgggggttacatccagaag |
| | | acagagcggaaaagtgggaagaggc |
| | | agactgagcgggaaaagaagaagaa |
| | | gattctggctgagaggaggaaggtg |
| | | ctggccattgaccacctgaatgaag |
| | | atcagctgagggagaaggccaagga |
| | | gctgtggcagagcatctataacttg |
| | | gaggcagagaagttcgacctgcagg |
| | | agaagttcaagcagcagaaatatga |
| | | gatcaatgttctccgaaacaggatc |
| | | aacgataaccagaaagtctccaaga |
| | | cccgcgggaaggctaaagtcaccgg |
| | | gcgctggaaatag |
| 167 | Buffer | tagtaatctagagcggccgcctcga |
| | Sequence | ggagctccggccg |
| 168 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 169 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

Construct 10 (“#6 KpnI-Des1-
coTNNT2-3UTR-pA”)

| | | |
|---|---|---|
| 170 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 171 | Buffer | Cctagttgttaatagtaatcaatta |
| | Sequence | cggggtcattagttcatagcccata |
| | Between ITR | tatggagttccg |
| | and Des 1 | |
| 172 | Des 1-promoter | CCCTGCCCCCACAGCTCCTCTCCTG |
| | | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 173 | Kozak | GCCACC |
| 174 | TNNT2 | ATGAGCGACATCGAGGAGGTGGTGG |
| | | AAGAGTACGAGGAAGAGGAACAGGA |
| | | GGAAGCCGCCGTGGAAGAAGAAGAG |
| | | GATTGGCGGGAAGATGAGGATGAAC |
| | | AGGAGGAAGCTGCTGAGGAGGACGC |
| | | CGAGGCCGAGGCAGAAACCGAGGAG |
| | | ACAAGAGCCGAAGAAGATGAGGAGG |
| | | AGGAAGAGGCCAAAGAGGCCGAGGA |
| | | CGGCCCCATGGAAGAGTCCAAGCCT |
| | | AAGCCCAGAAGCTTCATGCCTAACC |
| | | TGGTCCCACCTAAGATTCCTGATGG |
| | | AGAACGGGTGGACTTCGACGACATC |
| | | CACCGGAAGCGGATGGAAAAAGACC |
| | | TGAACGAGCTGCAGGCCCTGATCGA |
| | | GGCCCACTTCGAGAATCGGAAGAAG |
| | | GAAGAAGAGGAGCTCGTGTCTCTGA |
| | | AGGACAGAATCGAGCGGAGAAGAGC |
| | | CGAACGGGCCGAGCAACAAAGAATC |
| | | AGAAACGAGCGCGAGAAGGAGAGAC |
| | | AGAACAGACTGGCCGAGGAGAGAGC |
| | | TAGAAGAGAAGAAGAGGAAAACCGG |
| | | AGGAAGGCCGAGGACGAGGCCAGAA |
| | | AGAAAAAGGCCCTGAGCAACATGAT |
| | | GCATTTCGGCGGCTACATCCAGAAG |
| | | ACAGAGCGGAAAAGCGGCAAAAGAC |
| | | AGACCGAGCGTGAGAAAAAGAAGAA |
| | | GATCCTGGCTGAGCGGAGAAAAGTG |
| | | CTGGCCATCGACCACCTGAATGAGG |
| | | ATCAGCTGAGAGAAAAGGCCAAGGA |
| | | ACTGTGGCAGAGCATCTACAACCTG |
| | | GAAGCTGAAAAGTTTGACCTGCAGG |
| | | AGAAGTTCAAGCAGCAGAAGTATGA |
| | | GATCAACGTGCTGAGGAATAGAATC |
| | | AACGACAACCAGAAGGTTTCTAAGA |
| | | CCAGAGGCAAGGCCAAGGTGACCGG |
| | | CAGATGGAAGTGA |
| 175 | 3′ UTR | agcctggcctccttcaccaaagatc |
| | | tgctcctcgctcgcacctgcctccg |
| | | gcctgcactcccccagttcccgggc |
| | | cctcctgggcaccccaggcagctcc |
| | | tgtttggaaatggggagctggccta |
| | | gggggagccaccactcctgcctgcc |
| | | cccacacccactccacaccagtaat |
| | | aaaaagccaccacacactga |
| 176 | Buffer | tagtaatctagagcggccgcctcga |
| | Sequence | ggagctccggccg |
| 177 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 178 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

Construct 11 (“#7 KpnI-Des1-TNNT2-HGHint1-
TNNT2-pA”)

| | | |
|---|---|---|
| 179 | ITR-L | Ttggccactccctctctgcgcgctc |
| | (“ITRdeltaL”) | gctcgctcactgaggccgggcgacc |
| | | aaaggtcgcccgacgcccgggcttt |
| | | gcccgggcggcctcagtgagcgagc |
| | | gagcgcgcagagagggagtggcca |
| 180 | Buffer | Cctagttgttaatagtaatcaatta |
| | Sequence | cggggtcattagttcatagcccata |
| | Between ITR | tatggagttccg |
| | and Des 1 | |
| 181 | Des 1- | CCCTGCCCCCACAGCTCCTCTCCTG |
| | promoter | TGCCTTGTTTCCCAGCCATGCGTTC |
| | | TCCTCTATAAATACCCGCTCTGGTA |
| | | TTTGGGGTTGGCAGCTGTTGCTGCC |
| | | AGGGAGATGGTTGGGTTGACATGCG |
| | | GCTCCTGACAAAACACAAACCCCTG |
| | | GTGTGTGTGGGCGTGGGTGGTGTGA |
| | | GTAGGGGGATGAATCAGGGAGGGGG |
| | | CGGGGGACCCAGGGGGCAGGAGCCA |
| | | CACAAAGTCTGTGCGGGGGTGGGAG |
| | | CGCACATAGCAATTGGAAACTGAAA |
| | | GCTAATCAGACCCTTTCTGGAAATC |
| | | AGCCCACTGTTTATATACTTGAGGC |
| | | CCCACCCTCGAGATAACCAGGGCTG |
| | | AAAGAGGCCCGCCTGGGGGCTGCAG |
| | | ACATGCTTGCTGCCTGCCCTGGCGA |
| | | AGGATTGGCAGGCTTGCCCGTCACA |
| | | GGACCCCCGCTGGCTGACTCAGGGG |
| | | CGCAGGCCTCTTGCGGGGGAGCTGG |
| | | CCTCCCCGCCCCCACGGCCACGGGC |
| | | CGCCCTTTCCTGGCAGGACAGCGGG |
| | | ATCTTGCAGCTGTCAGGGGAGGGGA |
| | | GGCGGGGGCTGATGTCAGGAGGGAT |
| | | ACAAATAGTGCCGACGGCTGGGGGC |
| | | CCTGTCTCCCCTCGCCGCATCCACT |
| | | CTCCGGCCGGccgcctgcccgccgc |
| | | ctcctccgtgcgcccgccagcctcg |
| | | cccgcgccgtca |
| 182 | Kozak | GCCACC |
| 183 | TNNT2 | ATGatgtctgacatagaagaggtgg |
| | Fragment 1 | tggaagagtacgaggaggag |
| 184 | HGH Intron I | Gtaagcgcccctaaaatccctttgg |
| | | cacaatgtctcctgaggggagaggc |
| | | agcgacctgtagatgggacgggggc |
| | | actaaccctcagggtttggggttct |
| | | gaatgtgagtatcgccatctaagcc |
| | | cagtatttggccaatctcagaaagc |
| | | tcctggctccctggaggatggagag |
| | | agaaaaacaaacagctcctggagca |
| | | gggagagtgttggcctcttgctctc |
| | | cggctccctctgttgccctctggtt |
| | | tctccccag |
| 185 | TNNT2 | Gagcaggaagaagcagctgttgaag |
| | Fragment 2 | aagaggaggactggagagaggacga |
| | | agacgagcaggaggaggcagcggaa |
| | | gaggatgctgaagcagaggctgaga |
| | | ccgaggagaccagggcagaagaaga |
| | | tgaagaagaagaggaagcaaaggag |
| | | gctgaagatggcccaatggaggagt |
| | | ccaaaccaaagcccaggtcgttcat |
| | | gcccaacttggtgcctcccaagatc |
| | | cccgatggagagagagtggactttg |
| | | atgacatccaccggaagcgcatgga |
| | | gaaggacctgaatgagttgcaggcg |
| | | ctgatcgaggctcactttgagaaca |
| | | ggaagaaagaggaggaggagctcgt |
| | | ttctctcaaagacaggatcgagaga |
| | | cgtcgggcagagcgggccgagcagc |
| | | agcgcatccggaatgagcgggagaa |
| | | ggagcggcagaaccgcctggctgaa |
| | | gagagggctcgacgagaggaggagg |
| | | agaacaggaggaaggctgaggatga |
| | | ggcccggaagaagaaggctttgtcc |
| | | aacatgatgcattttgggggttaca |
| | | tccagaagacagagcggaaaagtgg |
| | | gaagaggcagactgagcgggaaaag |
| | | aagaagaagattctggctgagagga |
| | | ggaaggtgctggccattgaccacct |
| | | gaatgaagatcagctgagggagaag |
| | | gccaaggagctgtggcagagcatct |
| | | ataacttggaggcagagaagttcga |
| | | cctgcaggagaagttcaagcagcag |
| | | aaatatgagatcaatgttctccgaa |
| | | acaggatcaacgataaccagaaagt |
| | | ctccaagacccgcgggaaggctaaa |
| | | gtcaccgggcgctggaaatag |
| 186 | Buffer | tagtaatctagagcggccgcctcga |
| | Sequence | ggagctccggccg |
| 187 | bGH poly(A) | Ctgtgccttctagttgccagccatc |
| | Signal | tgttgtttgcccctcccccgtgcct |
| | | tccttgaccctggaaggtgccactc |
| | | ccactgtcctttcctaataaaatga |
| | | ggaaattgcatc |
| 188 | ITR-R | aggaacccctagtgatggagttggc |
| | | cactccctctctgcgcgctcgctcg |
| | | ctcactgaggccgggcgaccaaagg |
| | | tcgcccgacgcccgggctttgcccg |
| | | ggcggcctcagtgagcgagcgagcg |
| | | cgcagagagggagtggccaa |

### The Transgene

A transgene may be employed to correct, reduce, eliminate, or otherwise ameliorate gene deficiencies, which may include deficiencies in which normal genes are expressed at less than normal levels, are expressed at normal or near-normal levels but having a gene product with abnormal activity, or deficiencies in which the functional gene product is not expressed. In several embodiments, the transgene sequence encodes a therapeutic protein or polypeptide which is to be expressed in a host cell. Embodiments of the present disclosure also include using multiple transgenes.

Cardiac troponin T (TNNT2), a component of the troponin complex within the thin filament of the sarcomere which allows actomyosin interaction and contraction to occur in response to Ca²⁺. Mutations in or perturbations in the function of TNNT2 are causative of hypertrophic (HCM) and dilated cardiomyopathy (DCM).

### Regulatory Elements

In some embodiments, the rAAV vector comprises one or more regions comprising a sequence that facilitates expression of the heterologous nucleic acid, e.g., expression regulatory sequences operatively linked to the heterologous nucleic acid. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence. Numerous such sequences are known in the art.

Promoters that may be used in accordance with the present disclosure may comprise any promoter that can drive the expression of the transgenes in the heart of the subject. In some embodiments, the promoter may be a tissue-specific promoter. A “tissue-specific promoter”, as used herein, refers to promoters that can only function in a specific type of tissue, e.g., the heart. Thus, a “tissue-specific promoter” is not able to drive the expression of the transgenes in other types of tissues. In some embodiments, the promoter that may be used in accordance with the present disclosure is a cardiac-restricted promoter. Non-limiting examples of Tissue-specific promoters and/or regulatory elements that may be used include (1) desmin, creatine kinase, myogenin, alpha myosin heavy chain, and natriuretic peptide, specific for muscle cells, and (2) albumin, alpha-1-antitrypsin, hepatitis B virus core protein promoters, specific for liver cells. Non-limiting examples of cardiac-restricted promoter selected from cardiac troponin C, cardiac troponin I, and cardiac troponin T (cTnT). In treating cardiomyopathies as provided for herein, cardiac-restricted promoters are advantageous at least due to the reduced possibility of off-target expression of the transgene(s), thereby effectively increasing the delivered dose to the heart and enhancing therapy. Non-limiting examples of expression regulatory sequences include promoters, insulators, silencers, response elements, introns, enhancers, initiation sites, termination signals, and poly(A) tails. Any combination of such regulatory sequences is contemplated herein (e.g., a promoter and an enhancer).

Alternatively, the promoter may be, without limitation, a promoter from one of the following genes: α-myosin heavy chain gene, 6-myosin heavy chain gene, myosin light chain 2v (MLC-2v) gene, myosin light chain 2a gene, CARP gene, cardiac α-actin gene, cardiac m2 muscarinic acetylcholine gene, atrial natriuretic factor gene (ANF), cardiac sarcoplasmic reticulum Ca-ATPase gene, skeletal α-actin gene; or an artificial cardiac promoter derived from MLC-2v gene.

To achieve appropriate expression levels of the nucleic acid, protein, or polypeptide of interest, any of a number of promoters suitable for use in the selected host cell may be employed. The promoter may be, for example, a constitutive promoter, tissue-specific promoter, inducible promoter, or a synthetic promoter. For example, constitutive promoters of different strengths can be used. An rAAV vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of non-viral constitutive promoters include various housekeeping gene promoters, as exemplified by the β-actin promoter, including the chicken β-actin promoter (CBA).

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Synthetic promoters are also contemplated herein. A synthetic promoter may comprise, for example, regions of known promoters, regulatory elements, transcription factor binding sites, enhancer elements, repressor elements, and the like.

Enhancer elements can function in combination with other regulatory elements to increase the expression of a transgene. In several embodiments, the enhancer elements are upstream (positioned 5′) of the transgene. Non-limiting embodiments of enhancer elements include nucleotide sequences comprising, for example, a 100 base pair element from Simian virus 40 (SV40 late 2XUSE), a 35 base pair element from Human Immunodeficiency Virus 1 (HIV-1 USE), a 39 base pair element from ground squirrel hepatitis virus (GHV USE), a 21 base pair element from adenovirus (Adenovirus L3 USE), a 21 base pair element from human prothrombin (hTHGB USE), a 53 base pair element from human C2 complement gene (hC2 USE), truncations of any of the foregoing, and combinations of the foregoing. In some embodiments the enhancer is derived from the α-myosin heavy chain (αMHC) gene. In some embodiments the αMHC enhancer comprises a nucleic acid sequence having at least about 85%, about 90%, about 95%, about 96%, about 97%, about 98%, about 99%, or 100% sequence identity to:

SEQ ID NO: 100
CCTTCAGATTAAAAATAACTAAGGTAAGGGCCATGTGGGTAGGGGAGGT
GGTGTGAGACGGTCCTGTCTCTCCTCTATCTGCCCATCGGCCCTTTGGG
GAGGAGGAATGTGCCCAAGGACTAAAAAAAGGCCCTGGAGCCAGAGGGG
CGAGGGCAGCAGACCTTTCATGGGCAAACCTCAGGGCTGCTGTC,
or.

Non-limiting polyadenylation signals include nucleotide sequences comprising, for example, a 624 base pair polyadenylation signal from human growth hormone (hGH), a 135 base pair polyadenylation signal from simian virus 40 (sV40 late), a 49 base pair synthetic polyadenylation signal from rabbit beta-globin (SPA), a 250 base pair polyadenylation signal from bovine growth hormone (bGH), truncations of any of the foregoing, and combinations of the foregoing.

In some embodiments of the disclosed rAAV vectors, the two or more transgenes are operably controlled by a single promoter. In some embodiments, each of the two or more transgenes are operably controlled by a distinct promoter.

In some embodiments, the rAAV vectors of the present disclosure further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes.

In such embodiments, the proteins encoded by different transgenes are translated individually (i.e., versus translated as a fusion protein). In some embodiments, the rAAV vectors of the present disclosure comprise at least, in order from 5′ to 3′, a first adeno-associated virus (AAV) inverted terminal repeat (ITR) sequence, a promoter operably linked to a first transgene, an IRES operably linked to a second transgene, a polyadenylation signal, and a second AAV inverted terminal repeat (ITR) sequence.

In some embodiments, the rAAV vectors of the present disclosure further comprise a polyadenylation (pA) signal.

### Expression Cassette

The expression cassette is composed of, at a minimum, a transgene and its regulatory sequences. Where the cassette is designed to be expressed from a rAAV, the expression cassette further contains 5′ and 3′ AAV ITRs. These ITR's may be full-length, or one or both of the ITRs may be truncated. In one embodiment, the rAAV is pseudotyed, i.e., the AAV capsid is from a different source AAV than that the AAV which provides the ITRs. In one embodiment, the ITRs of AAV serotype 2 are used. In additional embodiments, the ITRs of AAV serotype 1 are used. However, ITRs from other suitable sources may be selected.

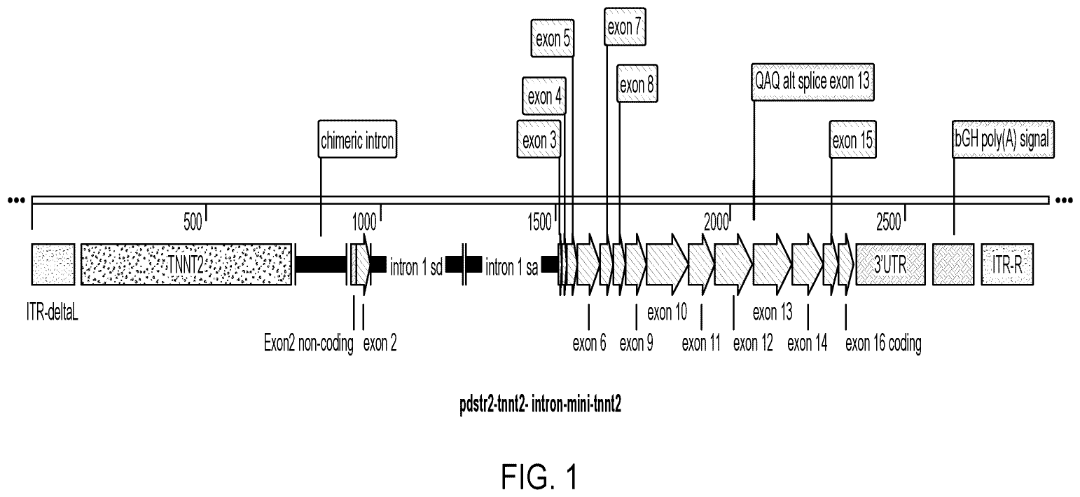

FIG. 1 depicts an embodiment of a construct described herein. At the 5′ end, an AAV ITR and TNNT2 promoter are present. A chimeric intron follows. Following the promoter, the TNNT2 transgene is depicted. The construct further includes a polyadenylated site following the TNNT2 transgene. Within the structural sequences described in the aforementioned construct, at least one or a plurality of spacer sequences (also referred to as buffer sequences) may be inserted at any point within the construct. Additionally, any number of promoter or regulatory sequences may comprise a construct to alter or change the expression of TNNT2.

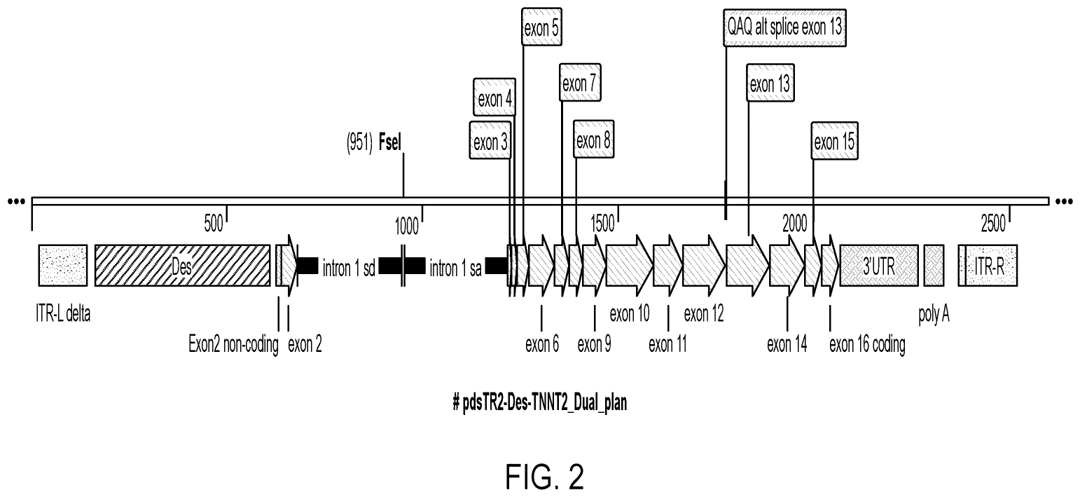

FIG. 2 depicts an embodiment of a construct described herein. At the 5′ end, an AAV ITR and Desmin promoter are present. A set of introns follows. Following the promoter, the TNNT2 transgene is depicted. The construct further includes a polyadenylated site following the TNNT2 transgene. Within the structural sequences described in the aforementioned construct, at least one or a plurality of spacer sequences may be inserted at any point within the construct. Additionally, any number of promoter or regulatory sequences may comprise a construct to alter or change the expression of TNNT2.

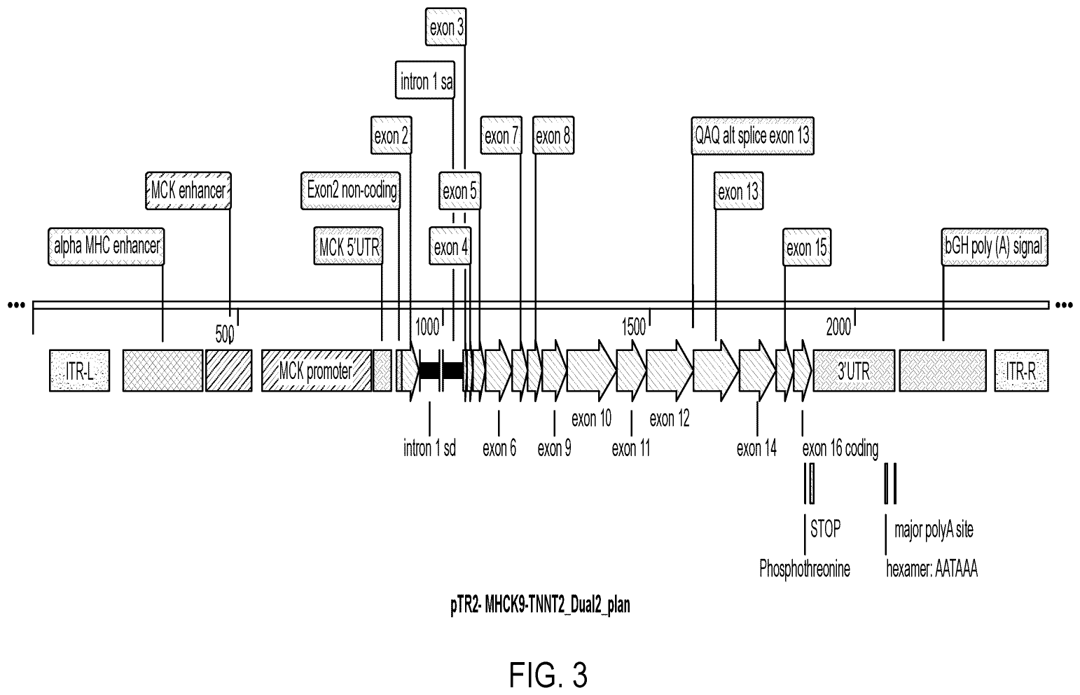

FIG. 3 depicts an embodiment of a construct described herein. At the 5′ end, an AAV ITR, alpha MHC enhancer, MCK9 enhancer, and MHCK9 (MCK) promoter are present. A set of introns follow. Following the promoter, the TNNT2 transgene is depicted. The construct further includes a polyadenylated site following the TNNT2 transgene. Within the structural sequences described in the aforementioned construct, at least one or a plurality of spacer sequences may be inserted at any point within the construct. Additionally, any number of promoter or regulatory sequences may comprise a construct to alter or change the expression of TNNT2.

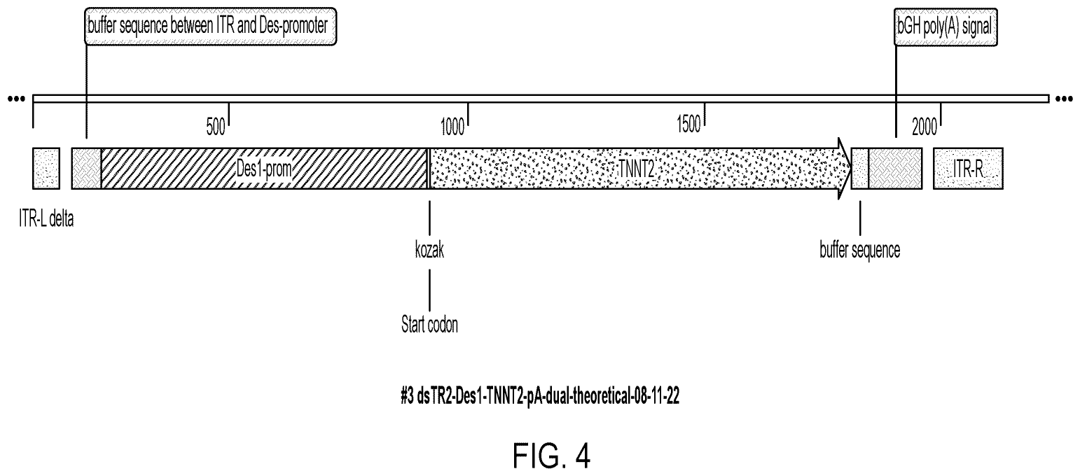

FIG. 4 depicts an embodiment of a construct described herein. At the 5′ end, an AAV ITR, and DES1 promoter are present. Following the promoter, the TNNT2 transgene and starting Kozak sequence are depicted. The construct further includes a polyadenylated site following the TNNT2 transgene. Within the structural sequences described in the aforementioned construct, at least one or a plurality of spacer sequences may be inserted at any point within the construct. Additionally, any number of promoter or regulatory sequences may comprise a construct to alter or change the expression of TNNT2.

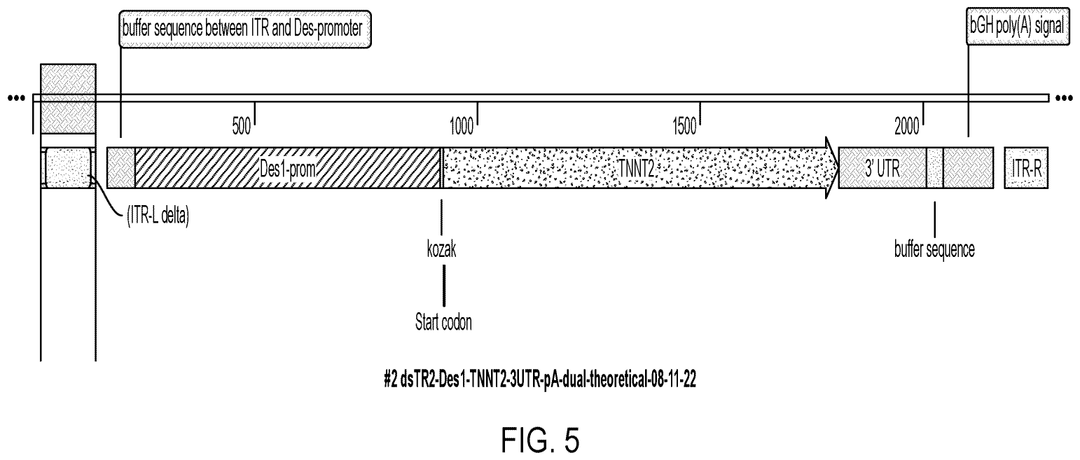

FIG. 5 depicts an embodiment of a construct described herein. At the 5′ end, an AAV ITR, and DES1 promoter are present. Following the promoter, the TNNT2 transgene and starting Kozak sequence are depicted. The construct further includes a 3′ UTR sequence, as well as a polyadenylated site following the TNNT2 transgene. Within the structural sequences described in the aforementioned construct, at least one or a plurality of spacer sequences may be inserted at any point within the construct. Additionally, any number of promoter or regulatory sequences may comprise a construct to alter or change the expression of TNNT2.

### Expression Cassette—Silencing Elements

Embodiments of this disclosure can provide compositions and methods for gene silencing and modulating protein expression using small nucleic acid molecules. Examples of nucleic acid molecules include molecules active in RNA interference (RNAi molecules), short interfering RNA (siRNA), double-stranded RNA (dsRNA), micro-RNA (miRNA), or short hairpin RNA (shRNA) molecules, as well as DNA-directed RNAs (ddRNA), Piwi-interacting RNAs (piRNA), or repeat associated siRNAs (rasiRNA). Such molecules are capable of mediating RNA interference against gene expression. In some embodiments, gene silencing can target a specific defective allele. In some embodiments, the gene silenced defective allele can then be replaced by a functional copy. In some embodiments, the functional copy of a gene is codon optimized, such that dissimilarities between a defective copy and a functional copy allow for silencing only of the defective copy.

In some embodiments, the expression cassette comprises a TNNT2 transgene and associated regulatory sequences, as well as a region capable of modulating endogenous TNNT2 gene expression, e.g., via a shRNA expression cassette. Attenuation, or knock down of endogenous gene expression can be accomplished using nucleotide sequences coding for small nucleic acid molecules, including shRNA. In some embodiments, the expression cassette comprises a transgene coding for a functional TNNT2 allele, as well as silencing elements to attenuate expression of a defective gene. In some embodiments, the silencing element is an intronic sequence within the overall construct. In some embodiments, the intronic sequence contains a restriction site. In some embodiments, the silencing element and intronic sequence can be utilized for subcloning in the expression cassette.

In some embodiments, delivery of nucleotide sequences can be separate from the vector encoding the expression cassette comprising a transgene and associated regulatory sequences. For example, two or more constructs may be co-administered, wherein at least one transgene construct comprises nucleic acid sequences encoding for a functional TNNT2 transgene, and wherein at least one other silencing construct comprises nucleic acid sequences for regulating endogenous TNNT2 gene expression. In some embodiments, administration of an expression cassette encoding a TNNT2 transgene is accompanied by, followed by, or preceded by, administration of a vector encoding a method for gene silencing or modulating TNNT2 protein expression.

In some embodiments, the expression cassette comprises a TNNT2 transgene and associated regulatory sequences, but does not include a region modulating endogenous TNNT2 gene expression. In some embodiments, a construct comprising the expression cassette with the functional TNNT2 transgene is administered. In some embodiments, the expression of the functional TNNT2 transgene is sufficient to provide therapeutic benefits to a subject.

### The Vector

Further provided herein are rAAV viral particles or rAAV preparations containing such particles. In several embodiments, rAAV particles comprise a viral capsid and one or more transgenes as described herein, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. In some embodiments, the rAAV particle is rAAV6 or rAAV9. In some embodiments, the rAAV particle is AAVrh74. In a preferred embodiment, the rAAV particle is AAVrh74. In an additional preferred embodiment, the rAAV is AAV9. In several embodiments, an rh74 AAV is mutated to advantageously enhance delivery to cardiac tissue, for example by a tryptophan to arginine mutation at amino acid 505 of VP1 capsid, or other mutations, as described in PCT Publication WO 2019/178412, which is incorporated in its entirety by reference herein. Non-limiting examples of derivatives, pseudotypes, and/or other vector types include, but are not limited to, AAVrh.10, AAVrh74, AAV2/1, AAV2/5, AAV2/6, AAV2/8, AAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AAVr3.45.

Such AAV serotypes and derivatives/pseudotypes, and methods of producing such derivatives/pseudotypes are known in the art (see, e.g., Mol Ther. 2012 Apr; 20 (4): 699-708. doi: 10.1038/mt.2011.287. Epub 2012 Jan. 24. The AAV vector toolkit: poised at the clinical crossroads. Asokan Al, Schaffer D V, Samulski R J.). In particular embodiments, the capsid of any of the herein disclosed rAAV particles is of the AAVrh.10 serotype. In a preferred embodiment, the capsid of the rAAV particle is AAVrh10 serotype. In some embodiments, the capsid is of the AAV2/6 serotype. In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al, J. Virol., 75:7662-7671, 2001; Halbert et al, J. Virol., 74:1524-1532, 2000; Zolotukhin et al, Methods, 28:158-167, 2002; and Auricchio et al., Hum. Molec. Genet., 10:3075-3081, 2001). rAAV Gene Therapy for Heart Diseases

In some embodiments, the rAAV vectors of the present disclosure further comprise a polyadenylation (pA) signal. For example, in preferred embodiments the pA signal comprises the following sequence: 29, 73, 97, or 123.

In some embodiments, the rAAV vectors of the present disclosure comprise at least, in order from 5′ to 3′, a first adeno-associated vims (AAV) inverted terminal repeat (ITR) sequence, a promoter operably linked to a transgene, a polyadenylation signal, and a second AAV inverted terminal repeat (ITR) sequence.

In some embodiments, the rAAV vector genome is circular. In some embodiments, the rAAV vector genome is linear. In some embodiments, the rAAV vector genome is single-stranded. In some embodiments, the rAAV vector genome is double-stranded. In some embodiments, the rAAV genome vector is a self-complementary rAAV vector.

Described herein are non-limiting examples of rAAV vectors. The vectors illustrated below comprise the linearized plasmid sequences set forth as SEQ ID NOs: 1-31, or 47-75, or SEQ ID NOs: 76-98, or SEQ ID NOs: 99-124, or SEQ ID NOs: 125-132, or SEQ ID NOs: 133-141 arranged in sequence. The vectors of the disclosure may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 1-31, or 47-75, or SEQ ID NOs: 76-98, or SEQ ID NOs: 99-124, or SEQ ID NOs: 125-132, or SEQ ID NOs: 133-141 arranged in sequence. In several embodiments, the rAAV has 100% identity to the sequences set forth as SEQ ID NOs: 1-31, or 47-75, or SEQ ID NOs: 76-98, or SEQ ID NOs: 99-124, or SEQ ID NOs: 125-132, or SEQ ID NOs: 133-141 arranged in sequence.

In some embodiments, any of the disclosed rAAV nucleic acid vector sequences comprise truncations at the 5′ or 3′ end relative to the sequences of any one of SEQ ID NOs: 1-31, or 47-75, or SEQ ID NOs: 76-98, or SEQ ID NOs: 99-124, or SEQ ID NOs: 125-132, or SEQ ID NOs: 133-141 arranged in sequence. In some embodiments, any of the rAAV vectors comprise a nucleotide sequence that differs from the sequence of any one of SEQ ID NOs: 1-31, or 47-75, or SEQ ID NOs: 76-98, or SEQ ID NOs: 99-124, or SEQ ID NOs: 125-132, or SEQ ID NOs: 133-141 arranged in sequence by 1, 2, 3, 4, 5, 6, 7, 8, 9, 10, 11, 12, 13, 14, 15, 16, 17, 18, or more than 18 nucleotides.

### Recombinant Adeno—Associated Virus Vectors and Therapeutic Use Thereof

Many serotypes of AAV have been cloned and sequenced. Serotypes 1 and 6 share >99% amino acid homology in their capsid proteins. Of the first six AAV serotypes, serotype 2 is widely characterized and therefore often used in gene transfer studies, however according to embodiments disclosed herein, other AAV serotypes are also used, such as AAV9, AAV20, AAVrh74, AAVrh10, and the like. In several embodiments, repeat administration of a given serotype that would be expected to elicit a humoral immune response is performed in connection with an immune management regimen. In several embodiments, an immune management regimen comprises administration of one or more agents that function as B-cell depletors, alone, or in conjunction with one or more agents that inhibit one or more aspects of the mTOR pathway. In one embodiment, an anti-CD20 antibody is administered and rapamycin is administered. In several embodiments, this allows for the repeat administration of a given serotype rAAV with reduced, limited or no immune response to a subsequent dosing of the rAAV. Further information about immune management can found in U.S. patent application Ser. No. 15/306,139, the entire contents of which is incorporated by reference herein.

The therapeutic rAAV vectors, therapeutic rAAV particles, or the composition comprising the therapeutic rAAV particles of the present disclosure, may be used for gene therapy for heart diseases in a human subject in need thereof, such as cardiomyopathies as provided for herein). Examples of heart disease that may be treated using the methods and compositions of the present disclosure include, but are not limited to, cardiomyopathy and acute ischemia. In some embodiments, cardiomyopathy is hypertrophic cardiomyopathy or dilated cardiomyopathy. In some embodiments, the cardiomyopathy is dilated cardiomyopathy and is caused by or associated with reduced or non-existent expression and/or function of TNNT2. In some embodiments, the cardiomyopathy is hypertrophic cardiomyopathy and is caused by or associated with reduced or non-existent expression and/or function of TNNT2. The therapeutic rAAV vectors, particles, and compositions comprising the therapeutic rAAV particles may be used for treatment of such heart failure (e.g., heart failure secondary to cardiomyopathy) when administered to a subject in need thereof, e.g., via vascular delivery into the coronary arteries and/or direct injection to the heart. The therapeutic rAAV vectors, particles, and compositions comprising the rAAV particles drive the concurrent expression of TNNT2 in the cardiomyocytes of the subject.

The amino acid sequence of the therapeutic TNNT2 encoded by the TNNT2 transgene is at least about 85%, at least 90%, at least 95%, at least 96%, at least 97%, at least 98% or at least 99% sequence identity to the amino acid sequence set forth as SEQ ID NO: 32-46 arranged in sequence.

In some embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications.

In accordance with some embodiments described herein, any of the sequences may be used, or a truncated or mutated form of any of the sequences disclosed herein (and/or included in the accompanying sequence listing) may be used and in any combination.

The promoter driving expression of the therapeutic nucleic acid can be, but is not limited to, a constitutive promoter, an inducible promoter, a tissue-specific promoter, a neuronal-specific promoter, a muscle-specific promoter, or a synthetic promoter. In some embodiments, the promoter is a neuronal-specific promoter or a muscle-specific promoter. A constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter. An inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline. A muscle-specific promoter can be, but is not limited to, desmin promoter, a creatine kinase promoter, a myogenin promoter, an alpha myosin heavy chain promoter, or a natriuretic peptide promoter.

In some embodiments, the therapeutic rAAV promoter comprises a neuronal- or cardiomuscle-specific promoter.

The therapeutic rAAV can be serotype 1, serotype 2, serotype 3, serotype 4, serotype 5, serotype 6, serotype 7, serotype 8, serotype 9, serotype 10, serotype 11, serotype 12, serotype rh10, or serotype rh74. The therapeutic rAAV can also be a pseudo-type rAAV.

In some embodiments, the therapeutic rAAV has a sequence sharing at least 85% sequence identity to SEQ ID NO: 1-31, or SEQ ID NOs: 76-98, or SEQ ID NOs: 99-124, or SEQ ID NOs: 125-132, or SEQ ID NOs: 133-141 arranged in sequence.

In some embodiments, the therapeutic rAAV has a sequence sharing at least 85% sequence identity to SEQ ID NO: 47-75 arranged in sequence.

In some embodiments, the therapeutic rAAV has a sequence sharing at least 95% sequence identity to SEQ ID NO: 1-31, or SEQ ID NOs: 76-98, or SEQ ID NOs: 99-124, or SEQ ID NOs: 125-132, or SEQ ID NOs: 133-141 arranged in sequence.

In some embodiments, the therapeutic rAAV has a sequence sharing at least 95% sequence identity to SEQ ID NO: 47-75 arranged in sequence.

### Pharmaceutical Formulations and Administration

Compositions described herein may further comprise a pharmaceutical excipient, buffer, or diluent, and may be formulated for administration to host cell ex vivo or in situ in an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., therapeutic rAAV particle or preparation) or more, although the percentage of the active ingredient(s) may, of course, be varied and may conveniently be between about 1 or 2% and about 70% or 90% or more of the weight or volume of the total formulation. Naturally, the amount of therapeutic agent(s) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art when preparing such pharmaceutical formulations. Additionally, a variety of dosages and treatment regimens may be desirable.

In certain circumstances, it will be desirable to deliver the therapeutic rAAV particles or preparations in suitably formulated pharmaceutical compositions disclosed herein; cither subcutaneously, intracardially, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells (e.g., cardiomyocytes and/or other heart cells), tissues, or organs. In some embodiments, the therapeutic rAAV particles or the composition comprising the therapeutic rAAV particles of the present invention are delivered systemically via intravenous injection, particularly in those for treating a human. In some embodiments, the therapeutic rAAV particles or the composition comprising the therapeutic rAAV particles of the present invention are injected directly into the heart of the subject. Direct injection to the heart may comprise injection into one or more of the myocardial tissues, the cardiac lining, or the skeletal muscle surrounding the heart, e.g., using a needle catheter. In several embodiments, direct injection to human heart is preferred, for example, if delivery is performed concurrently with a surgical procedure or interventional procedure whereby access to the heart is improved. In some embodiments, the interventional procedure includes any procedure wherein coronary or pulmonary perfusion is altered. In some embodiments, the interventional procedure includes one or more of percutaneous administration, catheterization, or coronary retroperfusion.

The pharmaceutical formulations of the compositions suitable for injectable use include sterile aqueous solutions or dispersions. In some embodiments, the formulation is sterile and fluid to the extent that easy syringability exists. In some embodiments, the form is stable under the conditions of manufacture and storage, and is preserved against the contaminating action of microorganisms, such as bacteria and fungi. The carrier may be a solvent or dispersion medium containing, for example, water, saline, ethanol, polyol (e.g., glycerol, propylene glycol, and liquid polyethylene glycol, and the like), suitable mixtures thereof, vegetable oils or other pharmaceutically acceptable carriers such as those that are Generally Recognized as Safe (GRAS) by the United States Food and Drug Administration. Proper fluidity may be maintained, for example, by the use of a coating, such as lecithin, by the maintenance of the required particle size in the case of dispersion and by the use of surfactants. In fact, there is virtually no limit to other components that may also be included, as long as the additional agents do not cause a significant adverse effect upon contact with the target cells or host tissues. The therapeutic rAAV particles or preparations may thus be delivered along with various other pharmaceutically acceptable agents as required in the particular instance. Such compositions may be purified from host cells or other biological sources, or alternatively may be chemically synthesized as described herein.

The amount of therapeutic rAAV particle or preparation, and/or therapeutic rAAV vector compositions and time of administration of such compositions will be within the purview of the skilled artisan having benefit of the present teachings. It is likely, however, that the administration of therapeutically-effective amounts of the compositions of the present disclosure may be achieved by a single administration, such as for example, a single injection of sufficient numbers of infectious particles to provide therapeutic benefit to the patient undergoing such treatment. In some circumstances, it may be desirable to provide multiple or successive administrations of the rAAV particle or preparation, and/or rAAV vector compositions, either over a relatively short, or a relatively prolonged period of time, as may be determined by the medical practitioner overseeing the administration of such compositions.

Toxicity and efficacy of the compositions utilized in methods of the present invention may be determined by standard pharmaceutical procedures, using either cells in culture or experimental animals to determine the LD₅₀ (the dose lethal to 50% of the population). The dose ratio between toxicity and efficacy the therapeutic index and it may be expressed as the ratio LD₅₀/ED₅₀. Those compositions that exhibit large therapeutic indices are preferred. While compositions that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

Other aspects of the present disclosure relate to methods and preparations for use with a subject, such as human or non-human subjects, a host cell in situ in a subject, or a host cell derived from a subject. In some embodiments, the subject is a mammal. In some embodiments, the subject is a companion animal. “A companion animal”, as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a human subject.

In some embodiments, one or more pharmaceutically acceptable excipients (including vehicles, carriers, diluents, and/or delivery polymers) are added to the pharmaceutical compositions including a therapeutic, thereby forming a pharmaceutical formulation suitable for in vivo delivery to a subject, such as a human.

A pharmaceutical composition or medicament includes a pharmacologically effective amount of at least one of the therapeutic and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents, and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

The rAAVs or pharmaceutical compositions as described herein, may be formulated for administration to host cell ex vivo or in situ in an animal, and particularly a human being. The rAAVs or pharmaceutical compositions can be administered by a variety of routes. Administration routes included, but are not limited to, intravenous, intra-arterial, subcutaneous, intramuscular, intrahepatic, intraperitoneal and/or local delivery to a target tissue. In some embodiments, a plurality of injections, or other administration types, are provided, for example 2, 3, 4, 5, 6, 7, 8, 9, 10 or more injections. Routes of administration may be combined, if desired. Depending on the embodiment, the first and second rAAV need not be administered the same number of times (e.g., the first rAAV may be administered 1 time, and the second vector may be administered three times). In some embodiments, the dosing is intramuscular administration.

In some embodiments, the number of rAAV particles administered to a subject may be on the order ranging from about 10⁶ to about 10¹⁴ particles/mL or about 10³ to about 10¹³ particles/mL, or any values in between for either range, such as for example, about 10⁶, 10⁷, 10⁸, 10⁹, 10¹⁰, 10¹¹, 10¹², 10¹³, or 10¹⁴ particles/mL. In some embodiments, the number of rAAV particles administered to a subject may be on the order ranging from about 10⁶ to about 10¹⁴ vector genomes (vgs)/mL or 10³ to 10¹⁵ vgs/mL, or any values in between for either range, such as for example, about 10⁶, 10⁷, 10⁸, 10⁹, 10¹⁰, 10¹¹, 10¹², 10¹³, or 10¹⁴ vgs/mL. The rAAV particles can be administered as a single dose, or divided into two or more administrations as may be required to achieve therapy of the particular disease or disorder being treated. In some embodiments, doses ranging from about 0.0001 mL to about 10 mLs are delivered to a subject.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see, for example, “Remington's Pharmaceutical Sciences” 15th Edition, pages 1035-1038 and 1570-1580). In several embodiments, the rAAV formulation will comprise, consist of, or consist essentially of active rAAV ingredient, a mono-basic buffer (e.g., sodium phosphate mono-basic buffer, a di-basic salt (e.g., sodium phosphate di-basic), a sodium-based tonicifier (e.g., sodium chloride tonicifier), a non-sodium tonicifier (e.g., magnesium chloride hexahydrate tonicifier), a surfactant (e.g., poloxamer 188 surfactant), and water. In several embodiments, the rAAV formulation will comprise, consist of, or consist essentially of active rAAV ingredient, sodium phosphate mono-basic buffer, sodium phosphate di-based, sodium chloride tonicifier, magnesium chloride hexahydrate tonicifier, poloxamer 188 surfactant, and water. In several embodiments, the active rAAV ingredient is present in the formulation according to the vector genome amounts provided for herein. In several embodiments, the mono-basic buffer (e.g., sodium phosphate mono-basic buffer) is present in the formulation at a concentration between about 0.2 mg/mL and about 0.5 mg/mL. In several embodiments, the di-basic salt (e.g., sodium phosphate di-basic) is present in the formulation at a concentration between about 1.5 mg/mL and about 4 mg/mL. In several embodiments, the sodium-based tonicifier (e.g., sodium chloride tonicifier) is present in the formulation at a concentration between about 8 mg/mL and about 12 mg/mL. In several embodiments, the non-sodium tonicifier (e.g., magnesium chloride hexahydrate tonicifier) is present in the formulation at a concentration between about 0.1 mg/mL and about 0.35 mg/mL. In several embodiments, the surfactant (e.g., poloxamer 188 surfactant) is present in the formulation at a concentration between about 0.05 mg/mL and about 0.8 mg/mL. In several embodiments, water is present to bring the volume of the formulation (e.g., a dosage unit) to 1 mL.

Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations in the required amount in the appropriate solvent with several of the other ingredients enumerated above, as required, followed by filtered sterilization. Generally, dispersions are prepared by incorporating the various sterilized active ingredients into a sterile vehicle that contains the basic dispersion medium and the other ingredients from those enumerated above. In the case of sterile powders for the preparation of sterile injectable solutions, the preferred methods of preparation are vacuum-drying and freeze-drying techniques, which yield a powder of the active ingredient plus any additional desired ingredient from a previously sterile-filtered solution thereof.

The amount of rAAV particle or preparation and time of administration of such particle or preparation will be within the purview of the skilled artisan having benefit of the present teachings. It is likely, however, that the administration of therapeutically-effective amounts of the rAAV particles or preparations of the present disclosure may be achieved by a single administration, such as for example, a single injection of sufficient numbers of infectious particles to provide therapeutic benefit to the patient undergoing such treatment. Alternatively, in some circumstances, it may be desirable to provide multiple or successive administrations of the rAAV particle or preparation, either over a relatively short, or a relatively prolonged period of time, as may be determined by the medical practitioner overseeing the administration of such compositions.

If desired, rAAV particles may be administered in combination with other agents as well, such as, e.g., proteins or polypeptides or various pharmaceutically-active agents, including one or more administrations of therapeutic polypeptides, biologically active fragments, or variants thereof. In fact, there is virtually no limit to other components that may also be included, as long as the additional agents do not cause a significant adverse effect upon contact with the target cells or host tissues. The rAAV particles or preparations may thus be delivered along with various other pharmaceutically acceptable agents as required in the particular instance. Such compositions may be purified from host cells or other biological sources, or alternatively may be chemically synthesized as described herein.

In some embodiments, treatment of a subject with a rAAV particles as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy.

As is apparent to those skilled in the art in view of the teachings of this specification, an effective amount of viral vector to be added can be empirically determined. Administration can be administered in a single dose, a plurality of doses, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

### Kits

Herein are described compositions including one or more of the disclosed rAAV vectors comprised within a kit for diagnosing, preventing, treating or ameliorating one or more symptoms of a heart disease or condition, such as a cardiomyopathy. Such kits may be useful in the diagnosis, prophylaxis, and/or therapy or a human disease, and may be particularly useful in the treatment, prevention, and/or amelioration of one or more symptoms of heart disease, such as a cardiomyopathy. In some embodiments, the heart disease is caused by cardiomyopathy. In some embodiments, the heart disease is caused by hypertrophic cardiomyopathy or dilated cardiomyopathy. In some embodiments, the heart disease is dilated cardiomyopathy or hypertrophic cardiomyopathy.

Kits comprising one or more of the disclosed rAAV vectors (as well as one or more virions, viral particles, transformed host cells or pharmaceutical compositions comprising such vectors); and instructions for using such kits in one or more therapeutic, diagnostic, and/or prophylactic clinical embodiments are also provided according to several embodiments. Such kits may comprise one or more reagents, restriction enzymes, peptides, therapeutics, pharmaceutical compounds, or means for delivery of the composition(s) to host cells, or to an animal (e.g., syringes, injectables, and the like). Depending on the embodiment, kits include those for treating, preventing, or ameliorating the symptoms of a disease, deficiency, dysfunction, and/or injury, or may include components for the large-scale production of the viral vectors themselves.

In some embodiments, a kit comprises one or more containers or receptacles comprising one or more doses of any of the described therapeutic. Such kits may be therapeutic in nature. In some embodiments, the kit contains a unit dosage, meaning a predetermined amount of a composition comprising, for example, a described therapeutic with or without one or more additional agents.

One or more of the components of a kit can be provided in one or more liquid or frozen solvents. The solvent can be aqueous or non-aqueous. The formulation in the kit can also be provided as dried powder(s) or in lyophilized form that can be reconstituted upon addition of an appropriate solvent.

In some embodiments, a kit comprises a label, marker, package insert, bar code and/or reader indicating directions of suitable usage of the kit contents. In some embodiments, the kit may comprise a label, marker, package insert, bar code and/or reader indicating that the kit contents may be administered in accordance with a certain dosage or dosing regimen to treat a subject.

In addition, a kit may also contain various reagents, including, but not limited to, wash reagents, elution reagents, and concentration reagents. Such reagents may be readily selected from among the reagents described herein, and from among conventional concentration reagents.

As used herein, the term “kit” may be used to describe variations of the portable, self-contained enclosure that includes at least one set of components to conduct one or more of the diagnostic or therapeutic methods of the invention.

### Combination Therapies

Multiple embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The compositions of the present disclosure may include rAAV particles or preparations, and/or rAAV vectors, either alone or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

If desired, rAAV particles may be administered in combination with other agents as well, such as, e.g., proteins or polypeptides or various pharmaceutically-active agents. This may, in some embodiments, reflect for example one or more administrations of therapeutic polypeptides, (e.g., a recombinant form of a functional peptide or protein that aids to replace or supplement the rAAV-based production of protein encoded by the transgene) biologically active fragments, or variants thereof. The rAAV particles or preparations may thus be delivered along with various other pharmaceutically acceptable agents as required in the particular instance. Such compositions may be purified from host cells or other biological sources, or alternatively may be chemically synthesized as described herein.

In some embodiments, the additional therapeutic agent comprises an anti-inflammatory agent. The anti-inflammatory agent can be, but is not limited to, a corticosteroid, cortisone hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, methylprednisolone, betamethasone, typically as betamethasone benzoate or betamethasone diproprionate; fluocinonide; prednisone; and triamcinolone, typically as triamcinolone acetonide. In some embodiments, the anti-inflammatory agent is a mast cell degranulation inhibitor, such as, without limitation, cromolyn (5,5′-(2-hydroxypropane-1,3-diyl)bis(oxy) bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-dicarboxylic acid) and tranilast (2-{[(2E)-3-(3,4-dimethoxyphenyl) prop-2-enoyl]amino}), and lodoxamide (2-[2-chloro-5-cyano-3-(oxaloamino) anilino]-2-oxoacetic acid). In some embodiments, the anti-inflammatory agent is a nonsteroidal anti-inflammatory drugs (NSAIDs), such as, without limitation, aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin.

In some embodiments, the anti-inflammatory agent comprises an antihistamine. The antihistamine can be, but is not limited to, clemastine, clemastine fumarate (2 (R)-[2-[1-(4-Chlorophenyl)-1-phenyl-ethoxy]ethyl-1-methylpyrrolidine), dexmedetomidine, doxylamine, loratidine, desloratidine and promethazine, and diphenhydramine, or pharmaceutically acceptable salts, solvates or esters thereof. In some embodiments, the antihistamine includes, without limitation, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. In some embodiments, the antihistamine is an H1 antagonist, such as, but not limited to, cetirizine, chlorpheniramine, dimenhydrinate, diphenhydramine, fexofenadine, hydroxyzine, orphenadrine, pheniramine, and doxylamine. In some embodiments, the antihistamine is an H2 antagonist, such as, but not limited to, cimetidine, famotidine, lafutidine, nizatidine, ranitidine, and roxatidine.

In some embodiments, the additional therapeutic agent comprises an antiviral agent, including antiretroviral agents. Suitable antiviral agents include, without limitation, remdesivir, acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, ribavirin and rimantadine.

In some embodiments, the additional therapeutic agent comprises an antibiotic. Non-limiting examples of suitable antibiotics include beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; Carbapenems such as imipenem and eropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tlimicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadmethoxine, sfulfamethazine, sulfaquinoxaline, sulfamerazine, sulfathiazole, sulfasalazine, sulfadiazine, sulfabromomethazine, suflaethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.)).

In some embodiments, the additional therapeutic agent comprises an antifungal agent, such as, but not limited to, itraconazole, ketoconazole, fluoconazole, and amphotericin B. In some embodiments, the therapeutic agent is an antiparasitic agents, such as, but not limited to, the broad spectrum antiparasitic medicament nitazoxanide; antimalarial drugs and other antiprotozoal agents (e.g., artemisins, mefloquine, lumefantrine, tinidazole, and miltefosine); anthelminthics such as mebendazole, thiabendazole, and ivermectin; and antiamoebic agents such as rifampin and amphotericin B.

In some embodiments, the additional therapeutic agent comprises an analgesic agent, including, without limitation, opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol; and nonopioid analgesics such as apazone, etodolac, diphenpyramide, indomethacin, meclofenamate, mefenamic acid, oxaprozin, phenylbutazone, piroxicam, and tolmetin.

The disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

Any titles or subheadings used herein are for organization purposes and should not be used to limit the scope of embodiments disclosed herein.

### EXAMPLES

The following examples are illustrative only and are not intended to be a limitation on the scope of the disclosure.

### Materials and Methods

Construct design. Constructs for expressing TNNT2 were engineered and codon optimized for expression in human tissues. Constructs are subcloned into a plasmid backbone suitable for production of AAV. The 5′ to 3′ construction of the synthetic TNNT2 genes are provided in Table 1. Constructs comprising single stranded AAV genomes were engineered to comprise the elements as provided in Tables 1˜4 below. Schematic representations of the mini-genes and constructs are provided in FIGS. 1 through 5. Certain constructs were engineered to comprise a unique restriction site (e.g., FseI) which is utilized for subcloning shRNA expression cassettes for knockdown of endogenous, mutant TNNT2 transcripts.

TABLE 1
Construct 1 (“pdsTR2-TNNT2-intron-mini-TNNT2”)

| SEQ ID: | Elements (5′→3′) | NT sequence |
|---|---|---|
| 1 | ITR-L | TTGGCCACTCCCTCTCTGCGCGCTCG |
| | (“ITRdeltaL”) | CTCGCTCACTGAGGCCGGGCGACCA |
| | | AAGGTCGCCCGACGCCCGGGCTTTGC |
| | | CCGGGCGGCCTCAGTGAGCGAGCGA |
| | | GCGCGCAGAGAGGGAGTGGCCA |
| 2 | Spacer | TCTAGAGGCGCGCCAAGCTT |
| 3 | TNNT2 promoter | GTCATGGAGAAGACCCACCTTGCAG |
| | | ATGTCCTCACTGGGGCTGGCAGAGCC |
| | | GGCAACCTGCCTAAGGCTGCTCAGTC |
| | | CATTAGGAGCCAGTAGCCTGGAAGA |
| | | TGTCTTTACCCCCAGCATCAGTTCAA |
| | | GTGGAGCAGCACATAACTCTTGCCCT |
| | | CTGCCTTCCAAGATTCTGGTGCTGAG |
| | | ACTTATGGAGTGTCTTGGAGGTTGCC |
| | | TTCTGCCCCCCAACCCTGCTCCCAGC |
| | | TGGCCCTCCCAGGCCTGGGTTGCTGG |
| | | CCTCTGCTTTATCAGGATTCTCAAGA |
| | | GGGACAGCTGGTTTATGTTGCATGAC |
| | | TGTTCCCTGCATATCTGCTCTGGTTTT |
| | | AAATAGCTTATCTGAGCAGCTGGAG |
| | | GACCACATGGGCTTATATGGCGTGGG |
| | | GTACATGATCCTGTAGCCTTGTCCCT |
| | | GGCACCTGCCAAAATAGCAGCCAAC |
| | | ACCCCCCACCCCCACCGCCATCCCCC |
| | | TGCCCCACCCGTCCCCTGTCGCACAT |
| | | TCCTCCCTCCGCAGGGCTGGCTCACC |
| | | AGGCCCCAGCCCACATGCCTGCTTAA |
| | | AGCCCTCTCCATCCTCTGCCTCACCC |
| | | AGTCCCCGCTGAGACTGAGCAGACG |
| | | CCTCCA |
| 4 | Spacer | AGATCTGAATTC |
| 5 | Chimeric intron | CAGGTAAGTATCAAGGTTACAAGAC |
| | | AGGTTTAAGGAGACCAATAGAAACT |
| | | GGGCTTGTCGAGACAGAGGGCCGGC |
| | | CAAGACTCTTGCGTTTCTGATAGGCA |
| | | CCTATTGGTCTTACTGACATCCACTTT |
| | | GCCTTTCTCTCCACAGGGT |
| 6 | Spacer | TCTAGAGGTACC |
| 7 | Exon 2 non-coding | GGAGAGCAGAGACC |
| 8 | Exon 2 | ATGTCTGACATAGAAGAGGTGGTGG |
| | | AAGAGTACGAGGAGGAG |
| 9 | TNNT2 intron 1 | GTGAGTATCTGGAGCATCTTGCCTGA |
| | with splice donor | GTGGGGTCCTCTCCCGCCGCTGCCCT |
| | | GACACCTGGTCCAGGAGCCTCCCAGC |
| | | TGTCCCTCGGATTCTGGGTAGAAGTA |
| | | GCTGTGTGTGTTTTGGGCACCCCGAG |
| | | GAGAGATATAGCCATTTCCTTTCCAC |
| | | TCCTGTACCCCTGGGTTGTAGATCCC |
| | | AGGCAGAGCACAGGCCGAGAGACTT |
| | | CGGGAAGCACAGAGGTTCAGCTCCA |
| | | GGGTCATCAGTGTCCTTGGCCAGTCT |
| | | TGGCTAG |
| 10 | FseI site | GGCCGGCC |
| 11 | TNNT2 intron 1 | AAGTGAGCAGAACGCTGGTGAGACC |
| | with splice acceptor | AAGCTGGCTGGGGCGGAGGCTTGCG |
| | | GAGGATGGTAGGAGGCAGCCCTGCA |
| | | TGAGAACGGCAGGCCAGGCTAGTGG |
| | | GTGTCATTGCAAGGTGGGCAGGGCA |
| | | GCGTGGACTCCACTAGGCAACAAGG |
| | | GAAAAGAAAGGGGGATTATCTTTGG |
| | | GGAAAGGCCAGTGTGTGCATGTGTGT |
| | | GCAGGCGTGTGTGTTTGCATGTGCTT |
| | | GTGTGCGAGCTACTGACAGTGTTTCC |
| | | TGTTGCTCTCAG |
| 12 | Exon 3 | GAGCAGGAA |
| 13 | Exon 4 | GAAGCAGCTGTTGAA |
| 14 | Exon 5 | GAAGAGGAGGACTGGAGAGAGGACG |
| | | AAGAC |
| 15 | Exon 6 | GAGCAGGAGGAGGCAGCGGAAGAG |
| | | GATGCTGAAGCAGAGGCTGAGACCG |
| | | AGGAGACCAGGGCAGAA |
| 16 | Exon 7 | GAAGATGAAGAAGAAGAGGAAGCA |
| | | AAGGAGGCTGAA |
| 17 | Exon 8 | GATGGCCCAATGGAGGAGTCCAAAC |
| | | CAAAGCCCAGG |
| 18 | Exon 9 | TCGTTCATGCCCAACTTGGTGCCTCC |
| | | CAAGATCCCCGATGGAGAGAGAGTG |
| | | GACTTTGAT |
| 19 | Exon 10 | GACATCCACCGGAAGCGCATGGAGA |
| | | AGGACCTGAATGAGTTGCAGGCGCT |
| | | GATCGAGGCTCACTTTGAGAACAGG |
| | | AAGAAAGAGGAGGAGGAGCTCGTTT |
| | | CTCTCAAAGACAGGATCGAG |
| 20 | Exon 11 | AGACGTCGGGCAGAGCGGGCCGAGC |
| | | AGCAGCGCATCCGGAATGAGCGGGA |
| | | GAAGGAGCGGCAGAACCGCCTGGCT |
| 21 | Exon 12 | GAAGAGAGGGCTCGACGAGAGGAGG |
| | | AGGAGAACAGGAGGAAGGCTGAGGA |
| | | TGAGGCCCGGAAGAAGAAGGCTTTG |
| | | TCCAACATGATGCATTTTGGGGGTTA |
| | | CATCCAGAAG |
| 22 | Exon 13 | ACAGAGCGGAAAAGTGGGAAGAGGC |
| | | AGACTGAGCGGGAAAAGAAGAAGAA |
| | | GATTCTGGCTGAGAGGAGGAAGGTG |
| | | CTGGCCATTGACCACCTGAATGAAGA |
| | | TCAGCTGAGG |
| 23 | Exon 14 | GAGAAGGCCAAGGAGCTGTGGCAGA |
| | | GCATCTATAACTTGGAGGCAGAGAA |
| | | GTTCGACCTGCAGGAGAAGTTCAAG |
| | | CAGCAGAAATATGAG |
| 24 | Exon 15 | ATCAATGTTCTCCGAAACAGGATCAA |
| | | CGATAACCAGAAAGTC |
| 25 | Exon 16 | TCCAAGACCCGCGGGAAGGCTAAAG |
| | | TCACCGGGCGCTGGAAA |
| 26 | Stop cassette | TAGTAATGA |
| 27 | TNNT2 3′ UTR | AGCCTGGCCTCCTTCACCAAAGATCT |
| | | GCTCCTCGCTCGCACCTGCCTCCGGC |
| | | CTGCACTCCCCCAGTTCCCGGGCCCT |
| | | CCTGGGCACCCCAGGCAGCTCCTGTT |
| | | TGGAAATGGGGAGCTGGCCTAGGTG |
| | | GGAGCCACCACTCCTGCCTGCCCCCA |
| | | CACCCACTCCACACCAGTAATAAAA |
| | | AGCCACCACACACTGA |
| 28 | Spacer | GCGGCCGCCTCGAGGAGCTCCGGCC |
| | | G |
| 29 | bGH poly(A) signal | CTGTGCCTTCTAGTTGCCAGCCATCT |
| | | GTTGTTTGCCCCTCCCCCGTGCCTTCC |
| | | TTGACCCTGGAAGGTGCCACTCCCAC |
| | | TGTCCTTTCCTAATAAAATGAGGAAA |
| | | TTGCATC |
| 30 | Spacer | GCATGTCGACGCGCCGGCGTCTAGA |
| 31 | ITR-R | AGGAACCCCTAGTGATGGAGTTGGC |
| | | CACTCCCTCTCTGCGCGCTCGCTCGC |
| | | TCACTGAGGCCGGGCGACCAAAGGT |
| | | CGCCCGACGCCCGGGCTTTGCCCGGG |
| | | CGGCCTCAGTGAGCGAGCGAGCGCG |
| | | CAGAGAGGGAGTGGCCAA |
| | | AA sequence |
| SEQ ID: | Elements (5′→3′) | (as applicable) |
| 32 | Exon 2 | MSDIEEVVEEYEEE |
| 33 | Exon 3 | EQE |
| 34 | Exon 4 | EAAVE |
| 35 | Exon 5 | EEEDWREDED |
| 36 | Exon 6 | EQEEAAEEDAEAEAE |
| | | TEETRAE |
| 37 | Exon 7 | EDEEEEEAKEAE |
| 38 | Exon 8 | DGPMEESKPKPR |
| 39 | Exon 9 | SFMPNLVPPKIPDGER |
| | | VDFD |
| 40 | Exon 10 | DIHRKRMEKDLNELQ |
| | | ALIEAHFENRKKEEEE |
| | | LVSLKDRIE |
| 41 | Exon 11 | RRRAERAEQQRIRNE |
| | | REKERQNRLA |
| 42 | Exon 12 | EERARREEEENRRKA |
| | | EDEARKKKALSNMM |
| | | HFGGYIQK |
| 43 | Exon 13 | TERKSGKRQTEREKK |
| | | KKILAERRKVLAIDHL |
| | | NEDQLR |
| 44 | Exon 14 | EKAKELWQSIYNLEA |
| | | EKFDLQEKFKQQKYE |
| 45 | Exon 15 | INVLRNRINDNQKV |
| 46 | Exon 16 | SKTRGKAKVTGRWK |

TABLE 2
Construct 2 (“pdsTR2-TNNT2-intron-mini-TNNT2”
without FseI site)

| SEQ | Elements | |
|---|---|---|
| ID: | (5′ -> 3′) | Nt sequence |
| 47 | ITR-L | TTGGCCACTCCCTCTCTGCGCGCTCG |
| | (“ITRdeltaL”) | CTCGCTCACTGAGGCCGGGCGACCAA |
| | | AGGTCGCCCGACGCCCGGGCTTTGCC |
| | | CGGGCGGCCTCAGTGAGCGAGCGAGC |
| | | GCGCAGAGAGGGAGTGGCCA |
| 48 | Spacer | TCTAGAGGCGCGCCAAGCTT |
| 49 | TNNT2 | GTCATGGAGAAGACCCACCTTGCAGA |
| | promoter | TGTCCTCACTGGGGCTGGCAGAGCCG |
| | | GCAACCTGCCTAAGGCTGCTCAGTCC |
| | | ATTAGGAGCCAGTAGCCTGGAAGATG |
| | | TCTTTACCCCCAGCATCAGTTCAAGT |
| | | GGAGCAGCACATAACTCTTGCCCTCT |
| | | GCCTTCCAAGATTCTGGTGCTGAGAC |
| | | TTATGGAGTGTCTTGGAGGTTGCCTT |
| | | CTGCCCCCCAACCCTGCTCCCAGCTG |
| | | GCCCTCCCAGGCCTGGGTTGCTGGCC |
| | | TCTGCTTTATCAGGATTCTCAAGAGG |
| | | GACAGCTGGTTTATGTTGCATGACTG |
| | | TTCCCTGCATATCTGCTCTGGTTTTA |
| | | AATAGCTTATCTGAGCAGCTGGAGGA |
| | | CCACATGGGCTTATATGGCGTGGGGT |
| | | ACATGATCCTGTAGCCTTGTCCCTGG |
| | | CACCTGCCAAAATAGCAGCCAACACC |
| | | CCCCACCCCCACCGCCATCCCCCTGC |
| | | CCCACCCGTCCCCTGTCGCACATTCC |
| | | TCCCTCCGCAGGGCTGGCTCACCAGG |
| | | CCCCAGCCCACATGCCTGCTTAAAGC |
| | | CCTCTCCATCCTCTGCCTCACCCAGT |
| | | CCCCGCTGAGACTGAGCAGACGCCTC |
| | | CA |
| 50 | Spacer | AGATCTGAATTC |
| 51 | Chimeric | CAGGTAAGTATCAAGGTTACAAGACA |
| | intron | GGTTTAAGGAGACCAATAGAAACTGG |
| | | GCTTGTCGAGACAGAGGGCCGGCCAA |
| | | GACTCTTGCGTTTCTGATAGGCACCT |
| | | ATTGGTCTTACTGACATCCACTTTGC |
| | | CTTTCTCTCCACAGGGT |
| 52 | Spacer | TCTAGAGGTACC |
| 53 | Exon 2 | GGAGAGCAGAGACC |
| | non-coding | |
| 54 | Exon 2 | ATGTCTGACATAGAAGAGGTGGTGGA |
| | | AGAGTACGAGGAGGAG |
| 55 | TNNT2 | GTGAGTATCTGGAGCATCTTGCCTGA |
| | intron 1 | GTGGGGTCCTCTCCCGCCGCTGCCCT |
| | | GACACCTGGTCCAGGAGCCTCCCAGC |
| | | TGTCCCTCGGATTCTGGGTAGAAGTA |
| | | GCTGTGTGTGTTTTGGGCACCCCGAG |
| | | GAGAGATATAGCCATTTCCTTTCCAC |
| | | TCCTGTACCCCTGGGTTGTAGATCCC |
| | | AGGCAGAGCACAGGCCGAGAGACTTC |
| | | GGGAAGCACAGAGGTTCAGCTCCAGG |
| | | GTCATCAGTGTCCTTGGCCAGTCTTG |
| | | GCTAGAAGTGAGCAGAACGCTGGTGA |
| | | GACCAAGCTGGCTGGGGCGGAGGCTT |
| | | GCGGAGGATGGTAGGAGGCAGCCCTG |
| | | CATGAGAACGGCAGGCCAGGCTAGTG |
| | | GGTGTCATTGCAAGGTGGGCAGGGCA |
| | | GCGTGGACTCCACTAGGCAACAAGGG |
| | | AAAAGAAAGGGGGATTATCTTTGGGG |
| | | AAAGGCCAGTGTGTGCATGTGTGTGC |
| | | AGGCGTGTGTGTTTGCATGTGCTTGT |
| | | GTGCGAGCTACTGACAGTGTTTCCTG |
| | | TTGCTCTCAG |
| 56 | Exon 3 | GAGCAGGAA |
| 57 | Exon 4 | GAAGCAGCTGTTGAA |
| 58 | Exon 5 | GAAGAGGAGGACTGGAGAGAGGACGA |
| | | AGAC |
| 59 | Exon 6 | GAGCAGGAGGAGGCAGCGGAAGAGGA |
| | | TGCTGAAGCAGAGGCTGAGACCGAGG |
| | | AGACCAGGGCAGAA |
| 60 | Exon 7 | GAAGATGAAGAAGAAGAGGAAGCAAA |
| | | GGAGGCTGAA |
| 61 | Exon 8 | GATGGCCCAATGGAGGAGTCCAAACC |
| | | AAAGCCCAGG |
| 62 | Exon 9 | TCGTTCATGCCCAACTTGGTGCCTCC |
| | | CAAGATCCCCGATGGAGAGAGAGTGG |
| | | ACTTTGAT |
| 63 | Exon 10 | GACATCCACCGGAAGCGCATGGAGAA |
| | | GGACCTGAATGAGTTGCAGGCGCTGA |
| | | TCGAGGCTCACTTTGAGAACAGGAAG |
| | | AAAGAGGAGGAGGAGCTCGTTTCTCT |
| | | CAAAGACAGGATCGAG |
| 64 | Exon 11 | AGACGTCGGGCAGAGCGGGCCGAGCA |
| | | GCAGCGCATCCGGAATGAGCGGGAGA |
| | | AGGAGCGGCAGAACCGCCTGGCT |
| 65 | Exon 12 | GAAGAGAGGGCTCGACGAGAGGAGGA |
| | | GGAGAACAGGAGGAAGGCTGAGGATG |
| | | AGGCCCGGAAGAAGAAGGCTTTGTCC |
| | | AACATGATGCATTTTGGGGGTTACAT |
| | | CCAGAAG |
| 66 | Exon 13 | ACAGAGCGGAAAAGTGGGAAGAGGCA |
| | | GACTGAGCGGGAAAAGAAGAAGAAGA |
| | | TTCTGGCTGAGAGGAGGAAGGTGCTG |
| | | GCCATTGACCACCTGAATGAAGATCA |
| | | GCTGAGG |
| 67 | Exon 14 | GAGAAGGCCAAGGAGCTGTGGCAGAG |
| | | CATCTATAACTTGGAGGCAGAGAAGT |
| | | TCGACCTGCAGGAGAAGTTCAAGCAG |
| | | CAGAAATATGAG |
| 68 | Exon 15 | ATCAATGTTCTCCGAAACAGGATCAA |
| | | CGATAACCAGAAAGTC |
| 69 | Exon 16 | TCCAAGACCCGCGGGAAGGCTAAAGT |
| | | CACCGGGCGCTGGAAA |
| 70 | Stop cassette | TAGTAATGA |
| 71 | TNNT2 3′ UTR | AGCCTGGCCTCCTTCACCAAAGATCT |
| | | GCTCCTCGCTCGCACCTGCCTCCGGC |
| | | CTGCACTCCCCCAGTTCCCGGGCCCT |
| | | CCTGGGCACCCCAGGCAGCTCCTGTT |
| | | TGGAAATGGGGAGCTGGCCTAGGTGG |
| | | GAGCCACCACTCCTGCCTGCCCCCAC |
| | | ACCCACTCCACACCAGTAATAAAAAG |
| | | CCACCACACACTGA |
| 72 | Spacer | GCGGCCGCCTCGAGGAGCTCCGGCCG |
| 73 | bGH poly(A) | CTGTGCCTTCTAGTTGCCAGCCATCT |
| | signal | GTTGTTTGCCCCTCCCCCGTGCCTTC |
| | | CTTGACCCTGGAAGGTGCCACTCCCA |
| | | CTGTCCTTTCCTAATAAAATGAGGAA |
| | | ATTGCATC |
| 74 | Spacer | GCATGTCGACGCGCCGGCGTCTAGA |
| 75 | ITR-R | AGGAACCCCTAGTGATGGAGTTGGCC |
| | | ACTCCCTCTCTGCGCGCTCGCTCGCT |
| | | CACTGAGGCCGGGCGACCAAAGGTCG |
| | | CCCGACGCCCGGGCTTTGCCCGGGCG |
| | | GCCTCAGTGAGCGAGCGAGCGCGCAG |
| | | AGAGGGAGTGGCCAA |

TABLE 3
Construct 3 (“pdsTR2-Des1-TNNT2 Dual Plan”)

| SEQ | Elements | |
|---|---|---|
| ID: | (5′ -> 3′) | NT sequence |
| 76 | ITR-L | TTGGCCACTCCCTCTCTGCGC |
| | (“ITRdeltaL”) | GCTCGCTCGCTCACTGAGGCC |
| | | GGGCGACCAAAGGTCGCCCGA |
| | | CGCCCGGGCTTTGCCCGGGCG |
| | | GCCTCAGTGAGCGAGCGAGCG |
| | | CGCAGAGAGGGAGTGGCCA |
| 77 | Des 1 | TAGACTAGCATGCTGCCCATG |
| | | TAAGGAGGCAAGGCCTGGGGA |
| | | CACCCGAGATGCCTGGTTATA |
| | | ATTAACCCAGACATGTGGCTG |
| | | CCCCCCCCCCCCCAACACCTG |
| | | CTGCCTCTAAAAATAACCCTG |
| | | CATGCCATGTTCCCGGCGAAG |
| | | GGCCAGCTGTCCCCCGCCAGC |
| | | TAGACTCAGCACTTAGTTTAG |
| | | GAACCAGTGAGCAAGTCAGCC |
| | | CTTGGGGCAGCCCATACAAGG |
| | | CCATGGGGCTGGGCAAGCTGC |
| | | ACGCCTGGGTCCGGGGTGGGC |
| | | ACGGTGCCCGGGCAACGAGCT |
| | | GAAAGCTCATCTGCTCTCAGG |
| | | GGCCCCTCCCTGGGGACAGCC |
| | | CCTCCTGGCTAGTCACACCCT |
| | | GTAGGCTCCTCTATATAACCC |
| | | AGGGGCACAGGGGCTGCCCTC |
| | | ATTCTACCACCACCTCCACAG |
| | | CACAGACAGACACTCAGGAGC |
| | | CAGCCAAA |
| 78 | Exon 2 Non- | GGAGAGCAGAGACC |
| | Coding | |
| 79 | Exon 2 | ATGTCTGACATAGAAGAGGTG |
| | | GTGGAAGAGTACGAGGAGGAG |
| 80 | Intron 1 sd | GTGAGTATCTGGAGCATCTTG |
| | | CCTGAGTGGGGTCCTCTCCCG |
| | | CCGCTGCCCTGACACCTGGTC |
| | | CAGGAGCCTCCCAGCTGTCCC |
| | | TCGGATTCTGGGTAGAAGTAG |
| | | CTGTGTGTGTTTTGGGCACCC |
| | | CGAGGAGAGATATAGCCATTT |
| | | CCTTTCCACTCCTGTACCCCT |
| | | GGGTTGTAGATCCCAGGCAGA |
| | | GCACAGGCCGAGAGACTTCGG |
| | | GAAGCACAGAGGTTCAGCTCC |
| | | AGGGTCATCAGTGTCCTTGGC |
| | | CAGTCTTGGCTAG |
| 81 | Intron 1 sa | AAGTGAGCAGAACGCTGGTGA |
| | | GACCAAGCTGGCTGGGGCGGA |
| | | GGCTTGCGGAGGATGGTAGGA |
| | | GGCAGCCCTGCATGAGAACGG |
| | | CAGGCCAGGCTAGTGGGTGTC |
| | | ATTGCAAGGTGGGCAGGGCAG |
| | | CGTGGACTCCACTAGGCAACA |
| | | AGGGAAAAGAAAGGGGGATTA |
| | | TCTTTGGGGAAAGGCCAGTGT |
| | | GTGCATGTGTGTGCAGGCGTG |
| | | TGTGTTTGCATGTGCTTGTGT |
| | | GCGAGCTACTGACAGTGTTTC |
| | | CTGTTGCTCTCAG |
| 82 | Exon 3 | GAGCAGGAA |
| 83 | Exon 4 | GAAGCAGCTGTTGAA |
| 84 | Exon 5 | GAAGAGGAGGACTGGAGAGAG |
| | | GACGAAGAC |
| 85 | Exon 6 | GAGCAGGAGGAGGCAGCGGAA |
| | | GAGGATGCTGAAGCAGAGGCT |
| | | GAGACCGAGGAGACCAGGGCA |
| | | GAA |
| 86 | Exon 7 | GAAGATGAAGAAGAAGAGGAA |
| | | GCAAAGGAGGCTGAA |
| 87 | Exon 8 | GATGGCCCAATGGAGGAGTCC |
| | | AAACCAAAGCCCAGG |
| 88 | Exon 9 | TCGTTCATGCCCAACTTGGTG |
| | | CCTCCCAAGATCCCCGATGGA |
| | | GAGAGAGTGGACTTTGAT |
| 89 | Exon 10 | GACATCCACCGGAAGCGCATG |
| | | GAGAAGGACCTGAATGAGTTG |
| | | CAGGCGCTGATCGAGGCTCAC |
| | | TTTGAGAACAGGAAGAAAGAG |
| | | GAGGAGGAGCTCGTTTCTCTC |
| | | AAAGACAGGATCGAG |
| 90 | Exon 11 | AGACGTCGGGCAGAGCGGGCC |
| | | GAGCAGCAGCGCATCCGGAAT |
| | | GAGCGGGAGAAGGAGCGGCAG |
| | | AACCGCCTGGCT |
| 91 | Exon 12 | GAAGAGAGGGCTCGACGAGAG |
| | | GAGGAGGAGAACAGGAGGAAG |
| | | GCTGAGGATGAGGCCCGGAAG |
| | | AAGAAGGCTTTGTCCAACATG |
| | | ATGCATTTTGGGGGTTACATC |
| | | CAGAAG |
| 92 | Exon 13 | ACAGAGCGGAAAAGTGGGAAG |
| | | AGGCAGACTGAGCGGGAAAAG |
| | | AAGAAGAAGATTCTGGCTGAG |
| | | AGGAGGAAGGTGCTGGCCATT |
| | | GACCACCTGAATGAAGATCAG |
| | | CTGAGG |
| 93 | Exon 14 | GAGAAGGCCAAGGAGCTGTGG |
| | | CAGAGCATCTATAACTTGGAG |
| | | GCAGAGAAGTTCGACCTGCAG |
| | | GAGAAGTTCAAGCAGCAGAAA |
| | | TATGAG |
| 94 | Exon 15 | ATCAATGTTCTCCGAAACAGG |
| | | ATCAACGATAACCAGAAAGTC |
| 95 | Exon 16 | TCCAAGACCCGCGGGAAGGCT |
| | | AAAGTCACCGGGCGCTGGAAA |
| | | TAG |
| 96 | 3′ UTR | AGCCTGGCCTCCTTCACCAAA |
| | | GATCTGCTCCTCGCTCGCACC |
| | | TGCCTCCGGCCTGCACTCCCC |
| | | CAGTTCCCGGGCCCTCCTGGG |
| | | CACCCCAGGCAGCTCCTGTTT |
| | | GGAAATGGGGAGCTGGCCTAG |
| | | GTGGGAGCCACCACTCCTGCC |
| | | TGCCCCCACACCCACTCCACA |
| | | CCAGTAATAAAAAGCCACCAC |
| | | ACACTGA |
| 97 | Poly A | AATAAAAGATCCTTATTTTCA |
| | | TTGGATCTGTGTGTTGGTTTT |
| | | TTGTGTG |
| 98 | ITR-R | AGGAACCCCTAGTGATGGAGT |
| | | TGGCCACTCCCTCTCTGCGCG |
| | | CTCGCTCGCTCACTGAGGCCG |
| | | GGCGACCAAAGGTCGCCCGAC |
| | | GCCCGGGCTTTGCCCGGGCGG |
| | | CCTCAGTGAGCGAGCGAGCGC |
| | | GCAGAGAGGGAGTGGCCAA |

TABLE 4
Construct 4 (“pTR2-MHCK9-TNNT2 Dual2 plan”)

| SEQ | Elements | |
|---|---|---|
| ID: | (5′ -> 3′) | NT sequence |
| 99 | ITR-L | TTGGCCACTCCCTCTCTGCGC |
| | (“ITRdeltaL”) | GCTCGCTCGCTCACTGAGGCC |
| | | GGGCGACCAAAGGTCGCCCGA |
| | | CGCCCGGGCTTTGCCCGGGCG |
| | | GCCTCAGTGAGCGAGCGAGCG |
| | | CGCAGAGAGGGAGTGGCCAAC |
| | | TCCATCACTAGGGGTTCCT |
| 100 | Alpha MHC | ACCCTTCAGATTAAAAATAAC |
| | enhancer | TGAGGTAAGGGCCTGGGTAGG |
| | | GGAGGTGGTGTGAGACGCTCC |
| | | TGTCTCTCCTCTATCTGCCCA |
| | | TCGGCCCTTTGGGGAGGAGGA |
| | | ATGTGCCCAAGGACTAAAAAA |
| | | AGGCCATGGAGCCAGAGGGGC |
| | | GAGGGCAACAGACCTTTCATG |
| | | GGCAAACCTTGGGGCCCTGCT |
| | | GT |
| 101 | MHCK9 (MCK) | CTGCCCATGTAAGGAGGCAAG |
| | Enhancer | GCCTGGGGACACCCGAGATGC |
| | | CTGGTTATAATTAACCCAGAC |
| | | ATGTGGCTGCCCCCCCCCCCC |
| | | CAACACCTGCTGCCTCTAAAA |
| | | ATAACC |
| 102 | MHCK9 (MCK) | GTTCCCGGCGAAGGGCCAGCT |
| | Promoter | GTCCCCCGCCAGCTAGACTCA |
| | | GCACTTAGTTTAGGAACCAGT |
| | | GAGCAAGTCAGCCCTTGGGGC |
| | | AGCCCATACAAGGCCATGGGG |
| | | CTGGGCAAGCTGCACGCCTGG |
| | | GTCCGGGGTGGGCACGGTGCC |
| | | CGGGCAACGAGCTGAAAGCTC |
| | | ATCTGCTCTCAGGGGCCCCTC |
| | | CCTGGGGACAGCCCCTCCTGG |
| | | CTAGTCACACCCTGTAGGCTC |
| | | CTCTATATAACCCAGGGGCAC |
| | | AGGGGCTGCCCTC |
| 103 | MHCK9 (MCK) | ACCACCACCTCCACAGCACAG |
| | 5′ UTR | ACAGACACTCAGGAGCAGCCA |
| | | G |
| 104 | Exon 2 Non- | GGAGAGCAGAGACC |
| | coding | |
| 105 | Exon 2 | ATGTCTGACATAGAAGAGGTG |
| | | GTGGAAGAGTACGAGGAGGAG |
| 106 | Intron 1 sd | GTGAGTATCTGGAGCATCTTG |
| | | CCTGAGTGGGGTCCTCTCCCG |
| | | CCGCTGCC |
| 107 | Intron 1 sa | TTGCATGTGCTTGTGTGCGAG |
| | | CTACTGACAGTGTTTCCTGTT |
| | | GCTCTCAG |
| 108 | Exon 3 | GAGCAGGAA |
| 109 | Exon 4 | GAAGCAGCTGTTGAA |
| 110 | Exon 5 | GAAGAGGAGGACTGGAGAGAG |
| | | GACGAAGAC |
| 111 | Exon 6 | GAGCAGGAGGAGGCAGCGGAA |
| | | GAGGATGCTGAAGCAGAGGCT |
| | | GAGACCGAGGAGACCAGGGCA |
| | | GAA |
| 112 | Exon 7 | GAAGATGAAGAAGAAGAGGAA |
| | | GCAAAGGAGGCTGAA |
| 113 | Exon 8 | GATGGCCCAATGGAGGAGTCC |
| | | AAACCAAAGCCCAGG |
| 114 | Exon 9 | TCGTTCATGCCCAACTTGGTG |
| | | CCTCCCAAGATCCCCGATGGA |
| | | GAGAGAGTGGACTTTGAT |
| 115 | Exon 10 | GACATCCACCGGAAGCGCATG |
| | | GAGAAGGACCTGAATGAGTTG |
| | | CAGGCGCTGATCGAGGCTCAC |
| | | TTTGAGAACAGGAAGAAAGAG |
| | | GAGGAGGAGCTCGTTTCTCTC |
| | | AAAGACAGGATCGAG |
| 116 | Exon 11 | AGACGTCGGGCAGAGCGGGCC |
| | | GAGCAGCAGCGCATCCGGAAT |
| | | GAGCGGGAGAAGGAGCGGCAG |
| | | AACCGCCTGGCT |
| 117 | Exon 12 | GAAGAGAGGGCTCGACGAGAG |
| | | GAGGAGGAGAACAGGAGGAAG |
| | | GCTGAGGATGAGGCCCGGAAG |
| | | AAGAAGGCTTTGTCCAACATG |
| | | ATGCATTTTGGGGGTTACATC |
| | | CAGAAG |
| 118 | Exon 13 | ACAGAGCGGAAAAGTGGGAAG |
| | | AGGCAGACTGAGCGGGAAAAG |
| | | AAGAAGAAGATTCTGGCTGAG |
| | | AGGAGGAAGGTGCTGGCCATT |
| | | GACCACCTGAATGAAGATCAG |
| | | CTGAGG |
| 119 | Exon 14 | GAGAAGGCCAAGGAGCTGTGG |
| | | CAGAGCATCTATAACTTGGAG |
| | | GCAGAGAAGTTCGACCTGCAG |
| | | GAGAAGTTCAAGCAGCAGAAA |
| | | TATGAG |
| 120 | Exon 15 | ATCAATGTTCTCCGAAACAGG |
| | | ATCAACGATAACCAGAAAGTC |
| 121 | Exon 16 | TCCAAGACCCGCGGGAAGGCT |
| | | AAAGTCACCGGGCGCTGGAAA |
| | | TAG |
| 122 | 3′ UTR | AGCCTGGCCTCCTTCACCAAA |
| | | GATCTGCTCCTCGCTCGCACC |
| | | TGCCTCCGGCCTGCACTCCCC |
| | | CAGTTCCCGGGCCCTCCTGGG |
| | | CACCCCAGGCAGCTCCTGTTT |
| | | GGAAATGGGGAGCTGGCCTAG |
| | | GTGGGAGCCACCACTCCTGCC |
| | | TGCCCCCACACCCACTCCACA |
| | | CCAGTAATAAAAAGCCACCAC |
| | | ACACTGA |
| 123 | BGH Poly A | CTGTGCCTTCTAGTTGCCAGC |
| | | CATCTGTTGTTTGCCCCTCCC |
| | | CCGTGCCTTCCTTGACCCTGG |
| | | AAGGTGCCACTCCCACTGTCC |
| | | TTTCCTAATAAAATGAGGAAA |
| | | TTGCATCGCATTGTCTGAGTA |
| | | GGTGTCATTCTATTCTGGGGG |
| | | GTGGGGTGGGGCAGGACAGCA |
| | | AGGGGGAGGATTGGGAAGACA |
| | | ATAGCAGGCATGCTGGGGA |
| 124 | ITR-R | AGGAACCCCTAGTGATGGAGT |
| | | TGGCCACTCCCTCTCTGCGCG |
| | | CTCGCTCGCTCACTGAGGCCG |
| | | GGCGACCAAAGGTCGCCCGAC |
| | | GCCCGGGCTTTGCCCGGGCGG |
| | | CCTCAGTGAGCGAGCGAGCGC |
| | | GCAGAGAGGGAGTGGCCAA |

AAV production. Recombinant AAV (rAAV) particles comprising each of the constructs are made by suspension transfection of Expi293F cells with the TNNT2 constructs and other plasmids needed for rAAV production (e.g., comprising rep and cap expression cassettes) to generate three groups of rAAV comprising (1) AAV9 capsid proteins; (2) rh74 capsid proteins; and (3) rh74 variant capsid proteins comprising a tryptophan to arginine mutation at amino acid 505 of the rh74 VP1 capsid protein. Vector is isolated using a capture column followed by an anion exchange column and purified using a cesium chloride gradient to a titer of 2-5E+13 vg/ml.

### Example 1. In Vitro Expression Study (Prophetic)

The three groups of rAAV comprising the TNNT2 constructs are made as described above and delivered to HEK293, C2C12 or cardiomyocytes derived from human induced pluripotent stem cells. Whole cell lysates are generated and probed for expression of TNNT2 by ELISA and/or immunoblotting and/or ddPCR.

### Example 2. In Vivo Expression Studies (Prophetic)

The three groups of rAAV comprising the TNNT2 constructs are made as described above and administered via the facial vein to newborn C57BL/6 mice (n=6-10/group) at 5E+13 vg/kg. Two to four weeks after rAAV dosing, heart, diaphragm and skeletal muscle tissues are harvested and whole cell lysates are analyzed for TNNT2 expression using ELISA and/or immunoblot and/or ddPCR. For constructs that also express shRNA cassettes for knockdown of the endogenous allele, levels of endogenous TNNT2 expression and/or levels of shRNA expression are analyzed.

In a separate experiment, the three groups of rAAV comprising the TNNT2 constructs are made as described above and administered via the jugular vein to 5-7 weeks old C57BL/6 mice (n=6-10/group) at three different doses: 1E+13 vg/kg, 5E+13 vg/kg or 1+E14 vg/kg. One month after rAAV dosing, heart, diaphragm and skeletal muscle tissues are harvested and whole cell lysates are analyzed for TNNT2 expression using ELISA and/or immunoblot and/or ddPCR. For constructs that also express shRNA cassettes for knockdown of the endogenous allele, levels of endogenous TNNT2 expression and/or levels of shRNA expression are analyzed.

### Example 3. Restoration of TNNT2 Expression In Vivo (Prophetic)

Familial cardiomyopathies are primary disorders of the myocardium caused by heritable mutations in single genes, such as cardiac troponin T (TNNT2), a component of the troponin complex within the thin filament of the sarcomere which allows actomyosin interaction and contraction to occur in response to Ca²⁺. Mutations in TNNT2 are causative of hypertrophic (HCM) and dilated cardiomyopathy (DCM). DCM is characterized by left ventricular or biventricular dilation and depressed myocardial contractility, with an incidence of 8/100,000, and prevalence of 36/100,000 (Ramratnam et al., (2016) Gene-Targeted Mice with the Human Troponin T R141W Mutation Develop Dilated Cardiomyopathy with Calcium Desensitization. PLOS ONE 11 (12): e0167681). Following the onset of symptoms, mortality is 25% at one year and 50% at 5 years.

In this Example, genetic mouse models of TNNT2-related cardiomyopathy are utilized to study the efficacy of the TNNT2 constructs provided herein. The models include heterozygous null animals (Tnnt2 (+/−)), transgenic mice which express disease relevant alleles (TG (K210Delta)), and mice harboring a knock in of the human TNNT2 R141W mutation which develop DCM with calcium desensitization (Ahmad et al., PLOS One. 2008 Jul. 9; 3 (7): e2642; Ramratnam et al., (2016) Gene-Targeted Mice with the Human Troponin T R141W Mutation Develop Dilated Cardiomyopathy with Calcium Desensitization. PLOS ONE 11 (12): e0167681)

rAAV comprising the TNNT2 constructs are made as described above and delivered via a single IV injection to presymptomatic and/or symptomatic the TNNT2 mouse models using different doses. Endpoints include survival as well as cardiac function monitored by echocardiography. Upon necropsy, heart tissues are collected and whole tissue lysates are analyzed for AAV biodistribution by ddPCR and for human TNNT2 expression by ELISA and/or immunoblot. In addition, tissue sections are analyzed for histopathology. Therapeutic effects of the rAAV are assessed via the measured endpoints and/or histopathology assessments.

### Example 4A. In Vitro Expression Analysis in Human and Murine Cell Lines

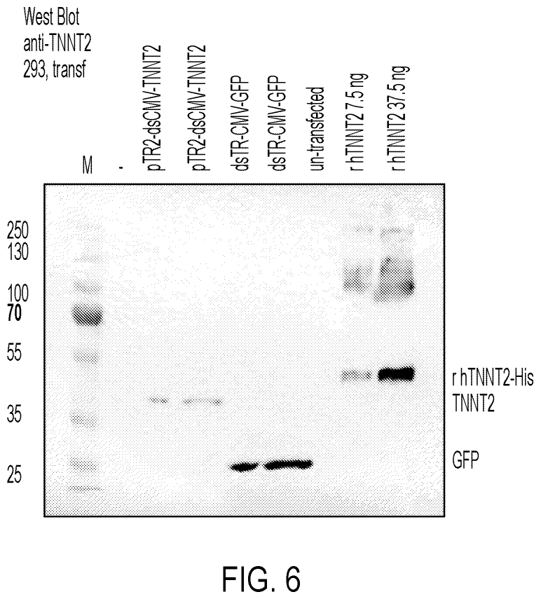

Human TNNT2 transgene expression was assessed using two distinct expression systems in two different cell types. The first system used a human TNNT2 expression construct driven by the ubiquitous CMV promoter. The construct was transfected into human HEK293 cells using TransfeX™ and 2.5 ug of plasmid DNA/well in a 6-well plate format at 80-90% confluency. The cells were incubated for approximately 48 hours then lysates were collected and processed with RIPA buffer containing 1 mM EDTA and protease/phosphatase inhibitors. A Pierce BCA protein assay was used to determine total protein concentration and equal protein amounts were loaded onto a Tris-Glycine gel along with appropriate controls, after denaturation at 99° C. for 5 mins with beta-mercaptoethanol. The proteins were transferred to a PVDF membrane, then probed with a human specific TNNT2 antibody (Abcam, cat #ab91605) at 1:1000 and secondary goat anti-rabbit-HRP antibody (Invitrogen, cat #31466). FIG. 6A is a Western blot of CMV-TNNT2 HEK293 lysates, showing protein bands for the CMV-TNNT2 constructs at the theoretical protein size of ˜37 kDa, with appropriate banding pattern of the positive and negative controls.

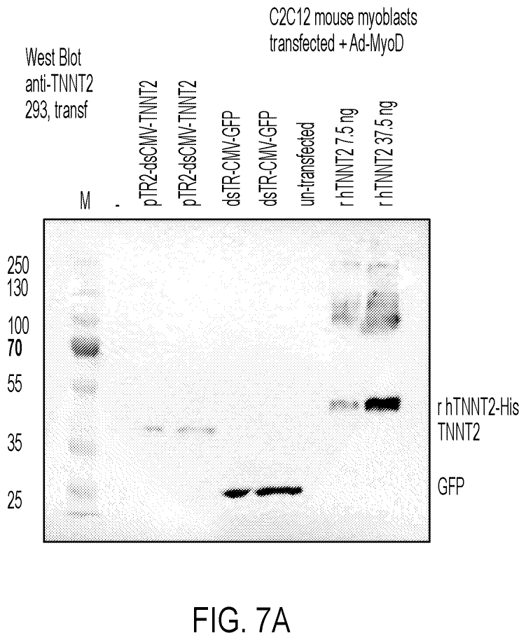

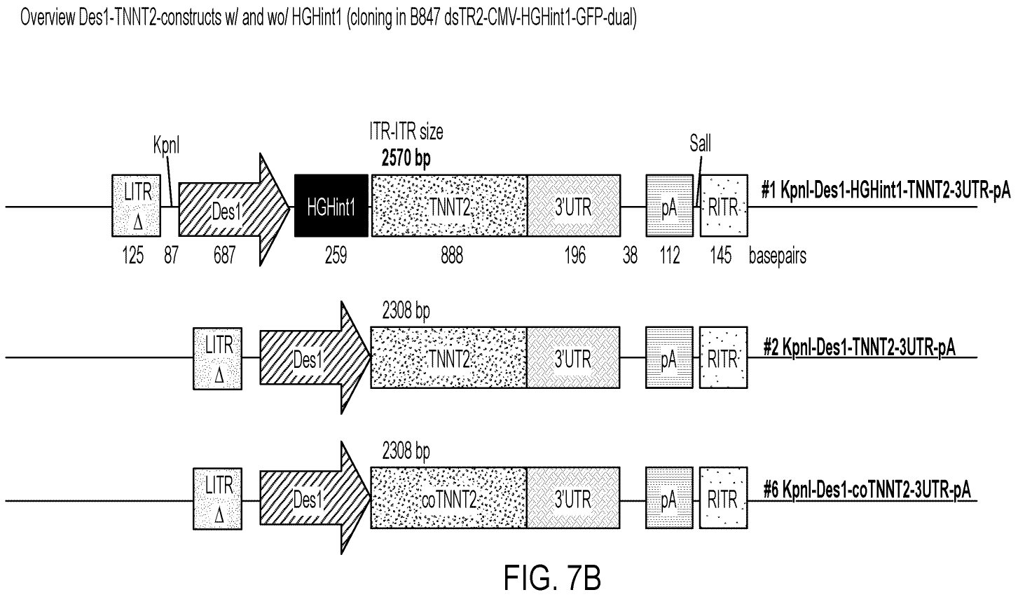

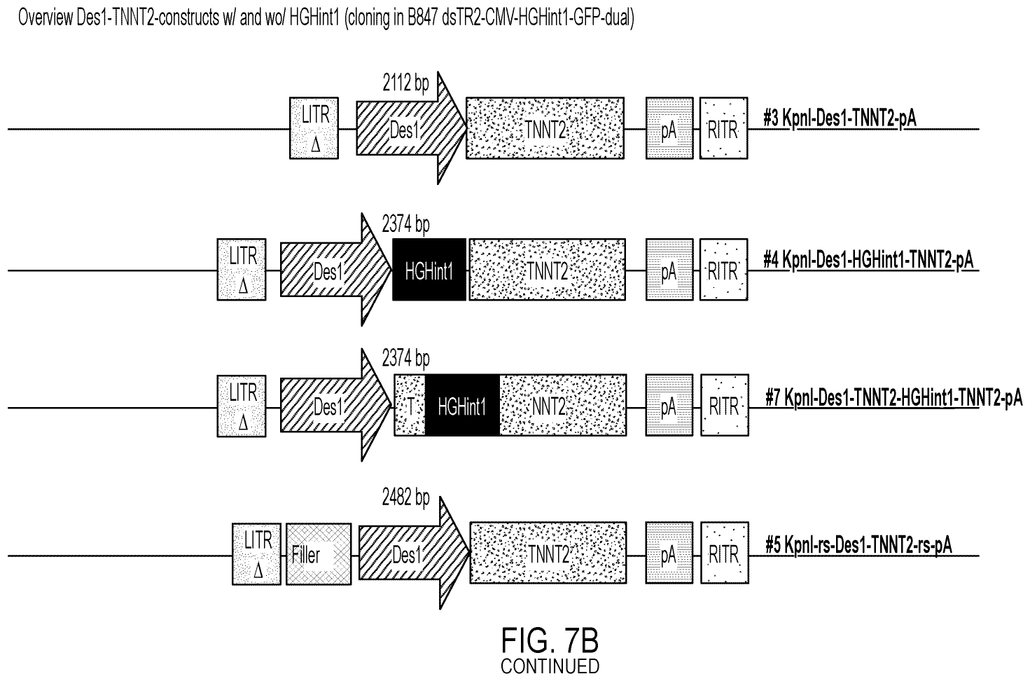

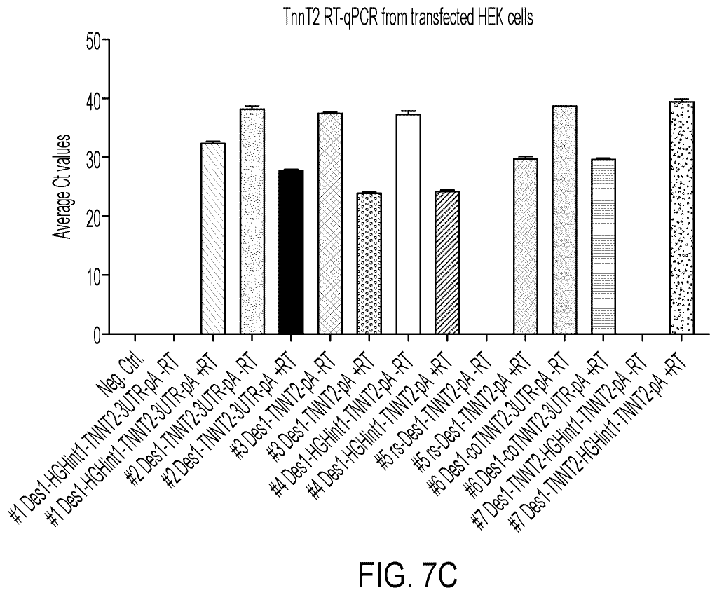

A second expression system used a human TNNT2 expression construct driven by the muscle selective promoter, Desmin. The construct was transfected into C2C12 mouse myoblasts with Ad-Myo-D helper infection to drive C2C12 differentiation. Cells were incubated for 48 hours, then lysates were collected and processed for Western blot and probed with anti-hTNNT2 primary antibody per above. FIG. 7A illustrates a Western blot of Des1-TNNT2 C2C12 lysates, where results show positive banding pattern at the expected theoretical protein size of approximately 37 kDa for Desmin-TNNT2 constructs, with no bands present in the negative control lanes.

Both human and murine in vitro data collectively support positive expression of the human TNNT2 constructs driven by two different promoters in multiple cell types, e.g., human and murine. According to several embodiments, human TNNT2 constructs as provided for herein are robustly expressed in human cells, such as human cardiomyocytes.

The objective of this study was to characterize transgene expression in adult WT mice after a single retro-orbital dose of two different AAVrh74-Des1-humanTNNT2 constructs, compared to AAV9-Des1-humanTNNT2 expression. Wild type C57BI/6 adult male mice were systemically administered AAVrh74-Des1-TNNT2-pA-dual, AAVrh74-Des1-TNNT2-3′UTR-pA-dual, or AAV9-Des1-TNNT2-pA-dual via the retro-orbital sinus in a single bolus dose of 5 ml/kg. The dose was administered at approximately 5 weeks of age and animals were sacrificed at 28-Days post dose according to the grouping shown in Table 5 below.

### Example 4B. In Vitro Expression Study

FIG. 7B illustrates exemplary gene maps for tested TNNT2 constructs. TNNT2 containing plasmids were transfected into HEK293 cells. Total mRNA was collected, with isolated total RNA treated with DNAseI to remove residual DNA. Total RNA was used as a template in an RT reaction to generate cDNA. qPCR results are illustrated in FIG. 7C. From FIG. 7C, the lower the average Ct value the greater the RNA expression. +RT samples are compared to the—RT negative controls. Samples were there is no bar indicate that there was no detection. TNNT2 RNA was detected in all +RT samples except for construct #7. These data indicate that all the constructs express a TNNT2 RNA.

### Example 5. In Vivo 28-Day Expression Study

The objective of this study was to characterize transgene expression in adult WT mice after a single retro-orbital dose of two different AAVrh74-Des1-humanTNNT2 constructs, compared to AAV9-Des1-humanTNNT2 expression. Wild type C57Bl/6 adult male mice were systemically administered AAVrh74-Des1-TNNT2-pA-dual, AAVrh74-Des1-TNNT2-3′UTR-pA-dual, or AAV9-Des1-TNNT2-pA-dual via the retro-orbital sinus in a single bolus dose of 5 ml/kg. The dose was administered at approximately 5 weeks of age and animals were sacrificed at 28-Days post dose according to the grouping shown in Table 5 below.

TABLE 5
Study Design Parameters

| | | | | | | Dosing | Terminal |
|---|---|---|---|---|---|---|---|
| Group | No. of | | Dose | Conc. | Dose | Regimen | Time |
| No. | Animals | Test Material | (vg/kg) | (vg/mL) | Volume | ROA | Point |
| 1 | 4 | Vehicle | 0 | 0 | 5 mL/kg | Intravenous - | Day 28 |
| 2 | 4 | AAV9-Des1- | 1.0E+13 | 2.07E12 | | Retro-orbital | |
| 3 | 4 | TNNT2-pA-dual | 5.0E+13 | 1.03E13 | | sinus | |
| 4 | 4 | AAV9-Des1- | 1.0E+13 | 2.04E12 | | | |
| 5 | 4 | TNNT2-3′UTR-pA- | 5.0E+13 | 1.02E13 | | | |
| | | dual | | | | | |
| 6 | 4 | AAVrh74-Des1- | 1.0E+13 | 2.06E12 | | | |
| 7 | 4 | TNNT2-pA-dual | 5.0E+13 | 1.01E13 | | | |
| 8 | 4 | AAVrh74-Des1- | 1.0E+13 | 2.05E12 | | | |
| 9 | 4 | TNNT2-3′UTR-pA- | 5.0E+13 | 1.04E13 | | | |
| | | dual | | | | | |

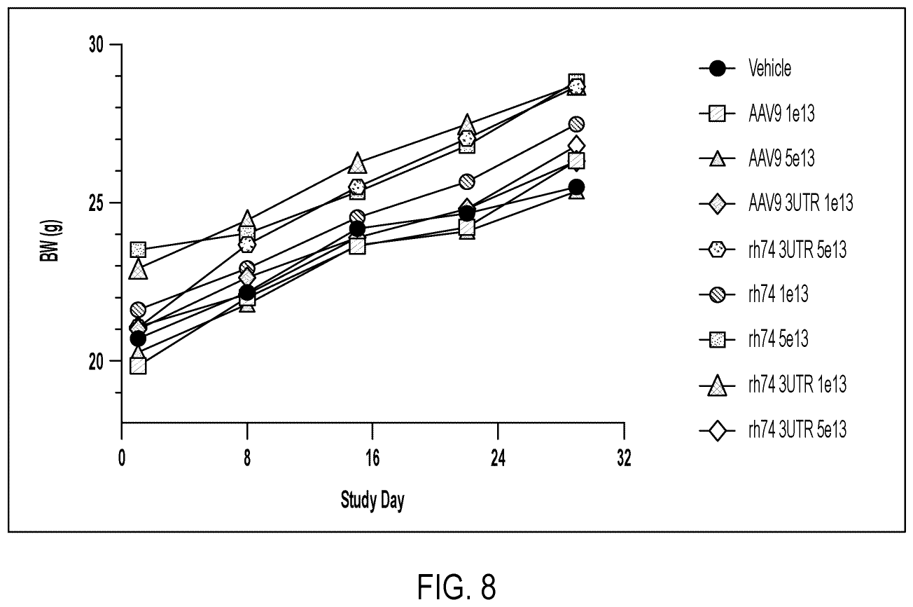

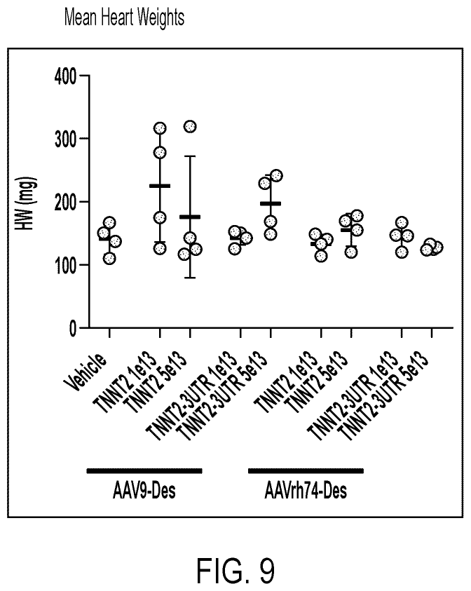

FIG. 8 illustrates measured body weights for each construct with data collected prior to dose, then weekly during the in-life phase and prior to termination. Whole heart weights were collected at termination for all animals as illustrated by FIG. 9. The data illustrate there were no significant differences in body weights or body weight gains in any treated group compared to controls. Observed variation in body weights through the duration of study were considered normal for this age and strain. All study animals survived to scheduled necropsy with no morbidity/mortality or clinical observations recorded on study. Upon scheduled necropsy, there were no gross or macroscopic observations noted, and all tissues were within historic normal ranges. Heart and liver were collected aseptically at necropsy and processed for endpoints of biodistribution and transgene mRNA expression.

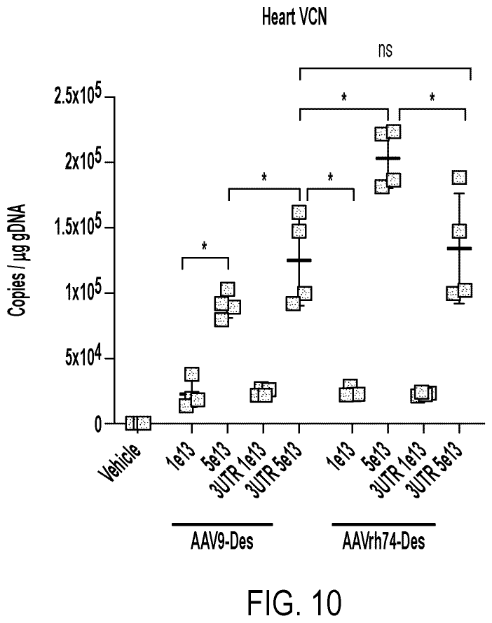

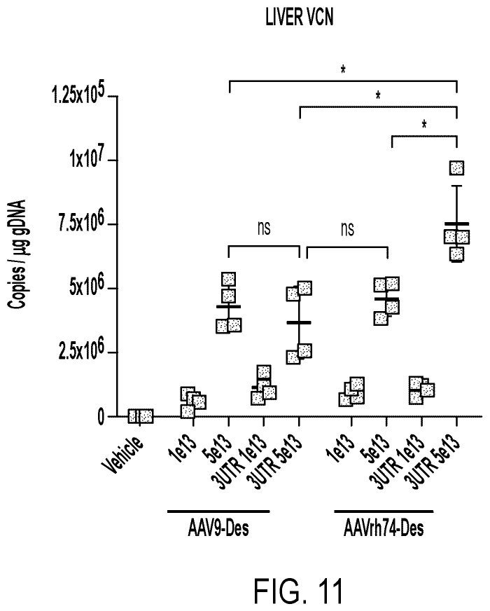

Tissue biodistribution analysis via ddPCR demonstrates a general dose response for each construct. As shown in FIG. 10, rh74-Des1-TNNT2-pA-dual construct at the high dose level of 5e13 vg/kg demonstrated a significantly greater vector copy number (“VCN”) in heart tissue compared to the same dose level of AAVrh74-Des1-TNNT2-3′UTR (p=0.0015), AAV9-Des1-TNNT2 (p<0.0001), or AAV9-Des1-TNNT2-3′UTR (p=0.0003). FIG. 11 illustrates liver biodistribution, with significantly greater vector copy number at 5e13 vg/kg for AAVrh74-Des1-TNNT2-3UTR compared to all other vectors at the same dose level. Liver biodistribution was not significantly different between AAV9-Des1-TNNT2, AAV9-Des1-TNNT2-3UTR, or AAVrh74-Des1-TNNT2 (FIG. 11). Vector copy number analysis results demonstrate more robust cardiac biodistribution of each of the non-limiting constructs tested, as compared to control. Additionally, the AAVrh74-Des1-TNNT2 construct shows particularly enhanced cardiac biodistribution compared to the same construct packaged in AAV9. In several embodiments, the enhanced cardiac biodistribution by the engineered TNNT2 constructs advantageously allows for tunability of transgene delivery. For example, if a given clinical scenario requires a greater delivery of a transgene, use of an AAVrh74-Des1-TNNT2 could be employed. This tunability, according to several embodiments, allows for a more flexible treatment paradigm and the ability to tailor AAV-based gene therapies employing constructs provided for herein to address specific patient needs.

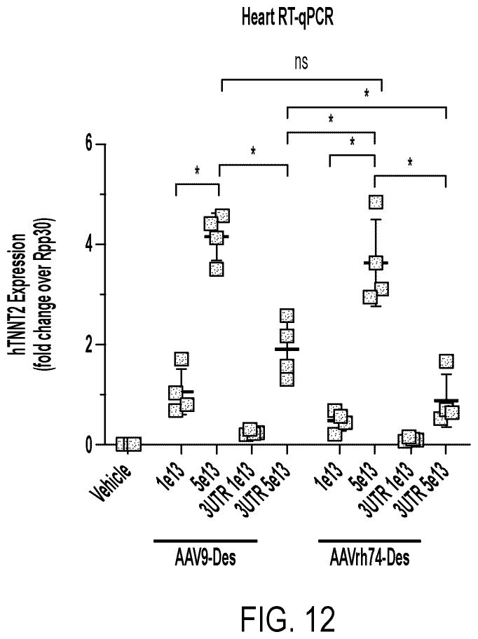

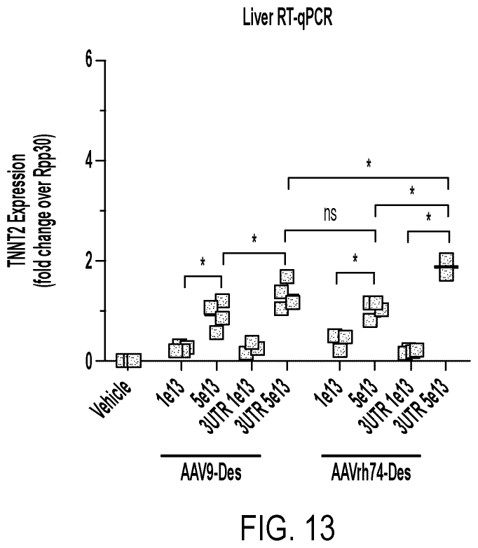

TNNT2 transgene mRNA expression was analyzed via RT-qPCR from all animals. FIG. 12 illustrates that there was significantly higher hTNNT2 transgene mRNA expression in heart tissue for AAVrh74-Des1-TNNT2 dosed at 5e13 vg/kg compared to the same dose level of AAVrh74-Des1-TNNT2-3UTR (p<0.0001) or AAV9-Des1-TNNT2-3UTR (p=0.0003); while there was no significant difference in heart expression levels compared to AAV9-Des1-TNNT2. FIG. 13 shows transgene mRNA expression in liver, which was approximately 2-2.5-fold lower than heart for all vectors at 5e13 vg/kg, with AAVrh74-Des1-TNNT2 demonstrating the lowest mRNA expression pairwise compared to other vectors at the same dose level. qPCR results demonstrate promoter driven tissue selective transgene expression.

Collectively, the results support that AAVrh74-Des1-TNNT2 drives enhanced cardiac selective transgene expression with concomitant reduced off-target tissue (liver) expression. In several embodiments, this allows for more efficient delivery of a vector as provided for herein, leading to enhanced therapeutic outcomes. The study data illustrates test article administration at the levels reported in this study were well-tolerated, did not result in adverse events or safety findings, and endpoint data follow a general dose-response relationship as expected.

### Example 6. Expression Through Use of a Consensus Kozak Sequence

An analysis of highly expressed genes in human heart tissues was performed to design a novel synthetic Kozak sequence to enhance transgene expression in the heart. Genes were selected from the Human Protein Atlas and Kozak sequences for each were identified in NCBI, is show in Table A below. A consensus sequence was derived using Weblogo (https://weblogo.berkeley.edu/logo.cgi). The consensus sequence (AGCCCCAAC (SEQ ID NO: 189)) was then utilized in the design of selected transgene constructs provided herein.

TABLE A

| | Kozak | SEQ |
|---|---|---|
| Gene | sequence | ID: |
| MYH7 | GGCACAGCC | 190 |
| ACTC1 | TGTGCCAAG | 191 |
| TNNI3 | AGTCTCAGC | 192 |
| MYL7 | GCAGAGAGA | 193 |
| NPPA | TCCAGAGAC | 194 |
| NPPB | TCCAGAGAC | 195 |
| TNNI2 | GACCTCAGG | 196 |
| MYBPC3 | TCTCTCAGG | 197 |
| MYL4 | CAAGACAAC | 198 |
| MYBPHL | AGGCCCAGC | 199 |
| MYH6 | AGCACCAAG | 200 |
| LRRC10 | AGCCTCCGC | 201 |
| ACTC1 | TGTGCCAAG | 202 |
| RD3L | AGGCTAAAA | 203 |
| Consensus | AGCCCCAAC | 204 |
| Sequence | | |

Self-complementary AAV (scAAV) genomes were designed with various promoters and alternative Kozak sequences, including the in silico derived sequence, as shown below.

1. scAAV with chick beta actin (CBA) promoter and
AGCGCCACC Kozak sequence:
Lower case = 5′ ITR
Underlined, uppercase = CBA promoter
Upper case, bold = Kozak sequence
Upper case = TNNT2 sequence
Upper case, bold underlined = PolyA
Lower case = 3′ WT ITR
SEQ ID NO: 205
ttggccactccctctctgcgcgctcgctcgctcactgaggccgggcgaccaaaggtcgcccgacgcccg
ggctttgcccgggggcctcagtgagcgagcgagcgcgcagagagggagtggccaactccatcactaggg
gttcctTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCGCGCCAG
GCGGGGCGGGGCGGGGCGAGGGGCGGGGCGGGGCGAGGCGGAGAGGTGCGGCGGCAGCCAATCAGAGCG
GCGCGCTCCGAAAGTTTCCTTTTATGGCGAGGCGGCGGCGGCGGCGGCCCTATAAAAAGCGAAGCGCGC
GGCGGGCGAGCGCCACCATGTCTGACATAGAAGAGGTGGTGGAAGAGTACGAGGAGGAGGAGCAGGAAG
AAGCAGCTGTTGAAGAAGAGGAGGACTGGAGAGAGGACGAAGACGAGCAGGAGGAGGCAGCGGAAGAGG
ATGCTGAAGCAGAGGCTGAGACCGAGGAGACCAGGGCAGAAGAAGATGAAGAAGAAGAGGAAGCAAAGG
AGGCTGAAGATGGCCCAATGGAGGAGTCCAAACCAAAGCCCAGGTCGTTCATGCCCAACTTGGTGCCTC
CCAAGATCCCCGATGGAGAGAGAGTGGACTTTGATGACATCCACCGGAAGCGCATGGAGAAGGACCTGA
ATGAGTTGCAGGCGCTGATCGAGGCTCACTTTGAGAACAGGAAGAAAGAGGAGGAGGAGCTCGTTTCTC
TCAAAGACAGGATCGAGAGACGTCGGGCAGAGCGGGCCGAGCAGCAGCGCATCCGGAATGAGCGGGAGA
AGGAGCGGCAGAACCGCCTGGCTGAAGAGAGGGCTCGACGAGAGGAGGAGGAGAACAGGAGGAAGGCTG
AGGATGAGGCCCGGAAGAAGAAGGCTTTGTCCAACATGATGCATTTTGGGGGTTACATCCAGAAGACAG
AGCGGAAAAGTGGGAAGAGGCAGACTGAGCGGGAAAAGAAGAAGAAGATTCTGGCTGAGAGGAGGAAGG
TGCTGGCCATTGACCACCTGAATGAAGATCAGCTGAGGGAGAAGGCCAAGGAGCTGTGGCAGAGCATCT
ATAACTTGGAGGCAGAGAAGTTCGACCTGCAGGAGAAGTTCAAGCAGCAGAAATATGAGATCAATGTTC
TCCGAAACAGGATCAACGATAACCAGAAAGTCTCCAAGACCCGCGGGAAGGCTAAAGTCACCGGGCGCT
GGAAATAGtagtaatctagagcggccgcctcgaggagctccggccgCTGTGCCTTCTAGTTGCCAGCCA
TCTGTTGTTTGCCCCTCCCCCGTGCCTTCCTTGACCCTGGAAGGTGCCACTCCCACTGTCCTTTCCTAA
TAAAATGAGGAAATTGCATCaggaacccctagtgatggagttggccactccctctctgcgcgctcgctc
gctcactgaggccgggcgaccaaaggtcgcccgacgcccgggctttgcccgggcggcctcagtgagcga
gcgagcgcgcagagagggagtggccaa
2. scAAV with chick beta actin (CBA) promoter and
in silico derived Kozak sequence:
Lower case = 5′ ITR
Underlined, uppercase = CBA promoter
Upper case, bold = in silico derived Kozak sequence
Upper case = TNNT2 cDNA
Upper case, bold underlined = PolyA
Lower case = 3′ WT ITR
SEQ ID NO: 206
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCGCGCCA
GGCGGGGCGGGGCGGGGCGAGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCCCTATAAAAAGCGAAGCGCG
CGGCGGGCGAGCCCCAACATGTCTGACATAGAAGAGGTGGTGGAAGAGTACGAGGAGGAGGAGCAGGAA
GAAGCAGCTGTTGAAGAAGAGGAGGACTGGAGAGAGGACGAAGACGAGCAGGAGGAGGCAGCGGAAGAG
GATGCTGAAGCAGAGGCTGAGACCGAGGAGACCAGGGCAGAAGAAGATGAAGAAGAAGAGGAAGCAAAG
GAGGCTGAAGATGGCCCAATGGAGGAGTCCAAACCAAAGCCCAGGTCGTTCATGCCCAACTTGGTGCCT
CCCAAGATCCCCGATGGAGAGAGAGTGGACTTTGATGACATCCACCGGAAGCGCATGGAGAAGGACCTG
AATGAGTTGCAGGCGCTGATCGAGGCTCACTTTGAGAACAGGAAGAAAGAGGAGGAGGAGCTCGTTTCT
CTCAAAGACAGGATCGAGAGACGTCGGGCAGAGCGGGCCGAGCAGCAGCGCATCCGGAATGAGCGGGAG
AAGGAGCGGCAGAACCGCCTGGCTGAAGAGAGGGCTCGACGAGAGGAGGAGGAGAACAGGAGGAAGGCT
GAGGATGAGGCCCGGAAGAAGAAGGCTTTGTCCAACATGATGCATTTTGGGGGTTACATCCAGAAGACA
GAGCGGAAAAGTGGGAAGAGGCAGACTGAGCGGGAAAAGAAGAAGAAGATTCTGGCTGAGAGGAGGAAG
GTGCTGGCCATTGACCACCTGAATGAAGATCAGCTGAGGGAGAAGGCCAAGGAGCTGTGGCAGAGCATC
TATAACTTGGAGGCAGAGAAGTTCGACCTGCAGGAGAAGTTCAAGCAGCAGAAATATGAGATCAATGTT
CTCCGAAACAGGATCAACGATAACCAGAAAGTCTCCAAGACCCGCGGGAAGGCTAAAGTCACCGGGCGC
TGGAAATAGtagtaatctagagcggccgcctcgaggagctccggccgCTGTGCCTTCTAGTTGCCAGCC
ATCTGTTGTTTGCCCCTCCCCCGTGCCTTCCTTGACCCTGGAAGGTGCCACTCCCACTGTCCTTTCCTA
ATAAAATGAGGAAATTGCATCaggaacccctagtgatggagttggccactccctctctgcgcgctcgct
cgctcactgaggccgggcgaccaaaggtcgcccgacgcccgggctttgcccgggcggcctcagtgagcg
agcgagcgcgcagagagggagtggccaa
3. scAAV with chick beta actin (CBA) promoter and
CAACCCAGC Kozak sequence:
Lower case = 5′ ITR
Underlined, uppercase = CBA promoter
Upper case, bold = Kozak sequence
Upper case = TNNT2 sequence
Upper case, bold underlined = PolyA
Lower case = 3′ WT ITR
SEQ ID NO: 207
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCGCGCCA
GGCGGGGCGGGGCGGGGCGAGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCCCTATAAAAAGCGAAGCGCG
CGGCGGGCGCAACCCAGCATGTCTGACATAGAAGAGGTGGTGGAAGAGTACGAGGAGGAGGAGCAGGAA
GAAGCAGCTGTTGAAGAAGAGGAGGACTGGAGAGAGGACGAAGACGAGCAGGAGGAGGCAGCGGAAGAG
GATGCTGAAGCAGAGGCTGAGACCGAGGAGACCAGGGCAGAAGAAGATGAAGAAGAAGAGGAAGCAAAG
GAGGCTGAAGATGGCCCAATGGAGGAGTCCAAACCAAAGCCCAGGTCGTTCATGCCCAACTTGGTGCCT
CCCAAGATCCCCGATGGAGAGAGAGTGGACTTTGATGACATCCACCGGAAGCGCATGGAGAAGGACCTG
AATGAGTTGCAGGCGCTGATCGAGGCTCACTTTGAGAACAGGAAGAAAGAGGAGGAGGAGCTCGTTTCT
CTCAAAGACAGGATCGAGAGACGTCGGGCAGAGCGGGCCGAGCAGCAGCGCATCCGGAATGAGCGGGAG
AAGGAGCGGCAGAACCGCCTGGCTGAAGAGAGGGCTCGACGAGAGGAGGAGGAGAACAGGAGGAAGGCT
GAGGATGAGGCCCGGAAGAAGAAGGCTTTGTCCAACATGATGCATTTTGGGGGTTACATCCAGAAGACA
GAGCGGAAAAGTGGGAAGAGGCAGACTGAGCGGGAAAAGAAGAAGAAGATTCTGGCTGAGAGGAGGAAG
GTGCTGGCCATTGACCACCTGAATGAAGATCAGCTGAGGGAGAAGGCCAAGGAGCTGTGGCAGAGCATC
TATAACTTGGAGGCAGAGAAGTTCGACCTGCAGGAGAAGTTCAAGCAGCAGAAATATGAGATCAATGTT
CTCCGAAACAGGATCAACGATAACCAGAAAGTCTCCAAGACCCGCGGGAAGGCTAAAGTCACCGGGCGC
TGGAAATAGtagtaatctagagcggccgcctcgaggagctccggccgCTGTGCCTTCTAGTTGCCAGCC
ATCTGTTGTTTGCCCCTCCCCCGTGCCTTCCTTGACCCTGGAAGGTGCCACTCCCACTGTCCTTTCCTA
ATAAAATGAGGAAATTGCATCaggaacccctagtgatggagttggccactccctctctgcgcgctcgct
cgctcactgaggccgggcgaccaaaggtcgcccgacgcccgggctttgcccgggcggcctcagtgagcg
agcgagcgcgcagagagggagtggccaa
4. scAAV with muscle creatine kinase (MCK) promoter
and AGCGCCACC Kozak sequence:
Lower case = 5′ ITR
Underlined, uppercase = MCK promoter
Upper case, bold = Kozak sequence
Upper case = TNNT2 sequence
Upper case, bold underlined = PolyA
Lower case = 3′ WT ITR
SEQ ID NO: 208
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctCAAGGCTGTGGGGGACTGAGGGCAGGCTGTAACAGGCTTGGGGGCCAGGGCTTATACGTGCC
TGGGACTCCCAAAGTATTACTGTTCCATGTTCCCGGCGAAGGGCCAGCTGTCCCCCGCCAGCTAGACTC
AGCACTTAGTTTAGGAACCAGTGAGCAAGTCAGCCCTTGGGGCAGCCCATACAAGGCCATGGGGCTGGG
CAAGCTGCACGCCTGGGTCCGGGGTGGGCACGGTGCCCGGGCAACGAGCTGAAAGCTCATCTGCTCTCA
GGGGCCCCTCCCTGGGGACAGCCCCTCCTGGCTAGTCACACCCTGTAGGCTCCTCTATATAACCCAGGG
GCACAGGGGCTGCCCTCAGCGCCACCATGTCTGACATAGAAGAGGTGGTGGAAGAGTACGAGGAGGAGG
AGCAGGAAGAAGCAGCTGTTGAAGAAGAGGAGGACTGGAGAGAGGACGAAGACGAGCAGGAGGAGGCAG
CGGAAGAGGATGCTGAAGCAGAGGCTGAGACCGAGGAGACCAGGGCAGAAGAAGATGAAGAAGAAGAGG
AAGCAAAGGAGGCTGAAGATGGCCCAATGGAGGAGTCCAAACCAAAGCCCAGGTCGTTCATGCCCAACT
TGGTGCCTCCCAAGATCCCCGATGGAGAGAGAGTGGACTTTGATGACATCCACCGGAAGCGCATGGAGA
AGGACCTGAATGAGTTGCAGGCGCTGATCGAGGCTCACTTTGAGAACAGGAAGAAAGAGGAGGAGGAGC
TCGTTTCTCTCAAAGACAGGATCGAGAGACGTCGGGCAGAGCGGGCCGAGCAGCAGCGCATCCGGAATG
AGCGGGAGAAGGAGCGGCAGAACCGCCTGGCTGAAGAGAGGGCTCGACGAGAGGAGGAGGAGAACAGGA
GGAAGGCTGAGGATGAGGCCCGGAAGAAGAAGGCTTTGTCCAACATGATGCATTTTGGGGGTTACATCC
AGAAGACAGAGCGGAAAAGTGGGAAGAGGCAGACTGAGCGGGAAAAGAAGAAGAAGATTCTGGCTGAGA
GGAGGAAGGTGCTGGCCATTGACCACCTGAATGAAGATCAGCTGAGGGAGAAGGCCAAGGAGCTGTGGC
AGAGCATCTATAACTTGGAGGCAGAGAAGTTCGACCTGCAGGAGAAGTTCAAGCAGCAGAAATATGAGA
TCAATGTTCTCCGAAACAGGATCAACGATAACCAGAAAGTCTCCAAGACCCGCGGGAAGGCTAAAGTCA
CCGGGCGCTGGAAATAGtagtaatctagagcggccgcctcgaggagctccggccgCTGTGCCTTCTAGT
TGCCAGCCATCTGTTGTTTGCCCCTCCCCCGTGCCTTCCTTGACCCTGGAAGGTGCCACTCCCACTGTC
CTTTCCTAATAAAATGAGGAAATTGCATCaggaacccctagtgatggagttggccactccctctctgcg
cgctcgctcgctcactgaggccgggcgaccaaaggtcgcccgacgcccgggctttgcccgggcggcctc
agtgagcgagcgagcgcgcagagagggagtggccaa
5. scAAV with muscle creatine kinase (MCK) promoter
and in silico derived Kozak sequence:
Lower case = 5′ ITR
Underlined, uppercase = MCK promoter
Upper case, bold = in silico derived Kozak sequence
Upper case = TNNT2 sequence
Upper case, bold underlined = PolyA
Lower case = 3′ WT ITR
SEQ ID NO: 209
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctCAAGGCTGTGGGGGACTGAGGGCAGGCTGTAACAGGCTTGGGGGCCAGGGCTTATACGTGCC
TGGGACTCCCAAAGTATTACTGTTCCATGTTCCCGGCGAAGGGCCAGCTGTCCCCCGCCAGCTAGACTC
AGCACTTAGTTTAGGAACCAGTGAGCAAGTCAGCCCTTGGGGCAGCCCATACAAGGCCATGGGGCTGGG
CAAGCTGCACGCCTGGGTCCGGGGTGGGCACGGTGCCCGGGCAACGAGCTGAAAGCTCATCTGCTCTCA
GGGGCCCCTCCCTGGGGACAGCCCCTCCTGGCTAGTCACACCCTGTAGGCTCCTCTATATAACCCAGGG
GCACAGGGGCTGCCCTCAGCCCCAACATGTCTGACATAGAAGAGGTGGTGGAAGAGTACGAGGAGGAGG
AGCAGGAAGAAGCAGCTGTTGAAGAAGAGGAGGACTGGAGAGAGGACGAAGACGAGCAGGAGGAGGCAG
CGGAAGAGGATGCTGAAGCAGAGGCTGAGACCGAGGAGACCAGGGCAGAAGAAGATGAAGAAGAAGAGG
AAGCAAAGGAGGCTGAAGATGGCCCAATGGAGGAGTCCAAACCAAAGCCCAGGTCGTTCATGCCCAACT
TGGTGCCTCCCAAGATCCCCGATGGAGAGAGAGTGGACTTTGATGACATCCACCGGAAGCGCATGGAGA
AGGACCTGAATGAGTTGCAGGCGCTGATCGAGGCTCACTTTGAGAACAGGAAGAAAGAGGAGGAGGAGC
TCGTTTCTCTCAAAGACAGGATCGAGAGACGTCGGGCAGAGCGGGCCGAGCAGCAGCGCATCCGGAATG
AGCGGGAGAAGGAGCGGCAGAACCGCCTGGCTGAAGAGAGGGCTCGACGAGAGGAGGAGGAGAACAGGA
GGAAGGCTGAGGATGAGGCCCGGAAGAAGAAGGCTTTGTCCAACATGATGCATTTTGGGGGTTACATCC
AGAAGACAGAGCGGAAAAGTGGGAAGAGGCAGACTGAGCGGGAAAAGAAGAAGAAGATTCTGGCTGAGA
GGAGGAAGGTGCTGGCCATTGACCACCTGAATGAAGATCAGCTGAGGGAGAAGGCCAAGGAGCTGTGGC
AGAGCATCTATAACTTGGAGGCAGAGAAGTTCGACCTGCAGGAGAAGTTCAAGCAGCAGAAATATGAGA
TCAATGTTCTCCGAAACAGGATCAACGATAACCAGAAAGTCTCCAAGACCCGCGGGAAGGCTAAAGTCA
CCGGGCGCTGGAAATAGtagtaatctagagcggccgcctcgaggagctccggccgCTGTGCCTTCTAGT
TGCCAGCCATCTGTTGTTTGCCCCTCCCCCGTGCCTTCCTTGACCCTGGAAGGTGCCACTCCCACTGTC
CTTTCCTAATAAAATGAGGAAATTGCATCaggaacccctagtgatggagttggccactccctctctgcg
cgctcgctcgctcactgaggccgggcgaccaaaggtcgcccgacgcccgggctttgcccgggcggcctc
agtgagcgagcgagcgcgcagagagggagtggccaa
6. scAAV with muscle creatine kinase (MCK) promoter
and CAACCCAGC Kozak sequence:
Lower case = 5′ ITR
Upper case, bold italics = spacer sequences
Underlined, uppercase = MCK promoter
Upper case, bold = Kozak sequence
Upper case = TNNT2 sequence
Upper case, bold underlined = PolyA
Lower case = 3′ WT ITR
SEQ ID NO: 210
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctAAGGCTGTGGGGGACTGAGGGCAGGCTGTAACAGGCTTGGGGGCCAGGGCTTATACGTGCCT
GGGACTCCCAAAGTATTACTGTTCCATGTTCCCGGCGAAGGGCCAGCTGTCCCCCGCCAGCTAGACTCA
GCACTTAGTTTAGGAACCAGTGAGCAAGTCAGCCCTTGGGGCAGCCCATACAAGGCCATGGGGCTGGGC
AAGCTGCACGCCTGGGTCCGGGGTGGGCACGGTGCCCGGGCAACGAGCTGAAAGCTCATCTGCTCTCAG
GGGCCCCTCCCTGGGGACAGCCCCTCCTGGCTAGTCACACCCTGTAGGCTCCTCTATATAACCCAGGGG
CACAGGGGCTGCCCTCCAACCCAGCATGTCTGACATAGAAGAGGTGGTGGAAGAGTACGAGGAGGAGGA
GCAGGAAGAAGCAGCTGTTGAAGAAGAGGAGGACTGGAGAGAGGACGAAGACGAGCAGGAGGAGGCAGC
GGAAGAGGATGCTGAAGCAGAGGCTGAGACCGAGGAGACCAGGGCAGAAGAAGATGAAGAAGAAGAGGA
AGCAAAGGAGGCTGAAGATGGCCCAATGGAGGAGTCCAAACCAAAGCCCAGGTCGTTCATGCCCAACTT
GGTGCCTCCCAAGATCCCCGATGGAGAGAGAGTGGACTTTGATGACATCCACCGGAAGCGCATGGAGAA
GGACCTGAATGAGTTGCAGGCGCTGATCGAGGCTCACTTTGAGAACAGGAAGAAAGAGGAGGAGGAGCT
CGTTTCTCTCAAAGACAGGATCGAGAGACGTCGGGCAGAGCGGGCCGAGCAGCAGCGCATCCGGAATGA
GCGGGAGAAGGAGCGGCAGAACCGCCTGGCTGAAGAGAGGGCTCGACGAGAGGAGGAGGAGAACAGGAG
GAAGGCTGAGGATGAGGCCCGGAAGAAGAAGGCTTTGTCCAACATGATGCATTTTGGGGGTTACATCCA
GAAGACAGAGCGGAAAAGTGGGAAGAGGCAGACTGAGCGGGAAAAGAAGAAGAAGATTCTGGCTGAGAG
GAGGAAGGTGCTGGCCATTGACCACCTGAATGAAGATCAGCTGAGGGAGAAGGCCAAGGAGCTGTGGCA
GAGCATCTATAACTTGGAGGCAGAGAAGTTCGACCTGCAGGAGAAGTTCAAGCAGCAGAAATATGAGAT
CAATGTTCTCCGAAACAGGATCAACGATAACCAGAAAGTCTCCAAGACCCGCGGGAAGGCTAAAGTCAC
CGGGCGCTGGAAATAGtagtaatctagagcggccgcctcgaggagctccggccgCTGTGCCTTCTAGTT
GCCAGCCATCTGTTGTTTGCCCCTCCCCCGTGCCTTCCTTGACCCTGGAAGGTGCCACTCCCACTGTCC
TTTCCTAATAAAATGAGGAAATTGCATCaggaacccctagtgatggagttggccactccctctctgcgc
gctcgctcgctcactgaggccgggcgaccaaaggtcgcccgacgcccgggctttgcccgggcggcctca
gtgagcgagcgagcgcgcagagagggagtggccaa
7. scAAV with TNNC1 promoter and AGCGCCACC Kozak
sequence:
Lower case = 5′ ITR
Underlined, uppercase = TNNC1 promoter
Upper case, bold = Kozak sequence
Upper case = TNNT2 sequence
Upper case, bold underlined = PolyA
Lower case = 3′ WT ITR
SEQ ID NO: 211
ttggccactccctctctgegcgctcgctcgctcactgaggccgggegaccaaaggtegcccgacgcccg
ggctttgcccgggcggcctcagtgagcgagcgagcgcgcagagagggagtggccaactccatcactagg
ggttcctGATCACTGGGACCAGAGGAGGGGCTGGAGGATACTACACGCAGGGGTGGGCTGGGCTGGGCT
GGGCTGGGCCAGGAATGCAGCGGGGCAGGGCTATTTAAGTCAAGGGCCGGCTGGCAACCCCAGCAAGCT
GTCCTGTGAGAGCGCCACCATGTCTGACATAGAAGAGGTGGTGGAAGAGTACGAGGAGGAGGAGCAGGA
AGAAGCAGCTGTTGAAGAAGAGGAGGACTGGAGAGAGGACGAAGACGAGCAGGAGGAGGCAGCGGAAGA
GGATGCTGAAGCAGAGGCTGAGACCGAGGAGACCAGGGCAGAAGAAGATGAAGAAGAAGAGGAAGCAAA
GGAGGCTGAAGATGGCCCAATGGAGGAGTCCAAACCAAAGCCCAGGTCGTTCATGCCCAACTTGGTGCC
TCCCAAGATCCCCGATGGAGAGAGAGTGGACTTTGATGACATCCACCGGAAGCGCATGGAGAAGGACCT
GAATGAGTTGCAGGCGCTGATCGAGGCTCACTTTGAGAACAGGAAGAAAGAGGAGGAGGAGCTCGTTTC
TCTCAAAGACAGGATCGAGAGACGTCGGGCAGAGCGGGCCGAGCAGCAGCGCATCCGGAATGAGCGGGA
GAAGGAGCGGCAGAACCGCCTGGCTGAAGAGAGGGCTCGACGAGAGGAGGAGGAGAACAGGAGGAAGGC
TGAGGATGAGGCCCGGAAGAAGAAGGCTTTGTCCAACATGATGCATTTTGGGGGTTACATCCAGAAGAC
AGAGCGGAAAAGTGGGAAGAGGCAGACTGAGCGGGAAAAGAAGAAGAAGATTCTGGCTGAGAGGAGGAA
GGTGCTGGCCATTGACCACCTGAATGAAGATCAGCTGAGGGAGAAGGCCAAGGAGCTGTGGCAGAGCAT
CTATAACTTGGAGGCAGAGAAGTTCGACCTGCAGGAGAAGTTCAAGCAGCAGAAATATGAGATCAATGT
TCTCCGAAACAGGATCAACGATAACCAGAAAGTCTCCAAGACCCGCGGGAAGGCTAAAGTCACCGGGCG
CTGGAAATAGtagtaatctagagcggccgcctcgaggagctccggccgCTGTGCCTTCTAGTTGCCAGC
CATCTGTTGTTTGCCCCTCCCCCGTGCCTTCCTTGACCCTGGAAGGTGCCACTCCCACTGTCCTTTCCT
AATAAAATGAGGAAATTGCATCaggaacccctagtgatggagttggccactccctctctgcgcgctcgc
tcgctcactgaggccgggcgaccaaaggtcgcccgacgcccgggctttgcccgggcggcctcagtgagc
gagcgagcgcgcagagagggagtggccaa
8. scAAV withTNNC1 promoter and in silico derived
Kozak sequence:
Lower case = 5′ ITR
Underlined, uppercase = TNNC1 promoter
Upper case, bold = in silico derived Kozak sequence
Upper case = TNNT2 sequence
Upper casc, bold underlined = PolyA
Lower case = 3′ WT ITR
SEQ ID NO: 212
ttggccactccctctctgcgcgctcgctcgctcactgaggccgggcgaccaaaggtcgcccgacgcccg
ggctttgcccgggcggcctcagtgagcgagcgagcgcgcagagagggagtggccaactccatcactagg
ggttcctGATCACTGGGACCAGAGGAGGGGCTGGAGGATACTACACGCAGGGGTGGGCTGGGCTGGGCT
GGGCTGGGCCAGGAATGCAGCGGGGCAGGGCTATTTAAGTCAAGGGCCGGCTGGCAACCCCAGCAAGCT
GTCCTGTGAGAGCCCCAACATGTCTGACATAGAAGAGGTGGTGGAAGAGTACGAGGAGGAGGAGCAGGA
AGAAGCAGCTGTTGAAGAAGAGGAGGACTGGAGAGAGGACGAAGACGAGCAGGAGGAGGCAGCGGAAGA
GGATGCTGAAGCAGAGGCTGAGACCGAGGAGACCAGGGCAGAAGAAGATGAAGAAGAAGAGGAAGCAAA
GGAGGCTGAAGATGGCCCAATGGAGGAGTCCAAACCAAAGCCCAGGTCGTTCATGCCCAACTTGGTGCC
TCCCAAGATCCCCGATGGAGAGAGAGTGGACTTTGATGACATCCACCGGAAGCGCATGGAGAAGGACCT
GAATGAGTTGCAGGCGCTGATCGAGGCTCACTTTGAGAACAGGAAGAAAGAGGAGGAGGAGCTCGTTTC
TCTCAAAGACAGGATCGAGAGACGTCGGGCAGAGCGGGCCGAGCAGCAGCGCATCCGGAATGAGCGGGA
GAAGGAGCGGCAGAACCGCCTGGCTGAAGAGAGGGCTCGACGAGAGGAGGAGGAGAACAGGAGGAAGGC
TGAGGATGAGGCCCGGAAGAAGAAGGCTTTGTCCAACATGATGCATTTTGGGGGTTACATCCAGAAGAC
AGAGCGGAAAAGTGGGAAGAGGCAGACTGAGCGGGAAAAGAAGAAGAAGATTCTGGCTGAGAGGAGGAA
GGTGCTGGCCATTGACCACCTGAATGAAGATCAGCTGAGGGAGAAGGCCAAGGAGCTGTGGCAGAGCAT
CTATAACTTGGAGGCAGAGAAGTTCGACCTGCAGGAGAAGTTCAAGCAGCAGAAATATGAGATCAATGT
TCTCCGAAACAGGATCAACGATAACCAGAAAGTCTCCAAGACCCGCGGGAAGGCTAAAGTCACCGGGCG
CTGGAAATAGtagtaatctagagcggccgcctcgaggagctccggccgCTGTGCCTTCTAGTTGCCAGC
CATCTGTTGTTTGCCCCTCCCCCGTGCCTTCCTTGACCCTGGAAGGTGCCACTCCCACTGTCCTTTCCT
AATAAAATGAGGAAATTGCATCaggaacccctagtgatggagttggccactccctctctgcgcgctcgc
tcgctcactgaggccgggcgaccaaaggtcgcccgacgcccgggctttgcccgggcggcctcagtgagc
gagcgagcgcgcagagagggagtggccaa
9.scAAV with TNNC1 promoter and CAACCCAGC Kozak
sequence:
Lowcr casc = 5′ ITR
Upper case, bold italics = spacer sequences
Underlined, uppercase = TNNC1 promoter
Upper case, bold = Kozak sequence
Upper case = TNNT2 sequence
Upper case, bold underlined = PolyA
Lower case = 3′ WT ITR
SEQ ID NO: 213
ttggccactccctctctgcgcgctcgctcgctcactgaggccgggcgaccaaaggtcgcccgacgcccg
ggctttgcccgggggcctcagtgagcgagcgagcgcgcagagagggagtggccaactccatcactaggg
gttcctGATCACTGGGACCAGAGGAGGGGCTGGAGGATACTACACGCAGGGGTGGGCTGGGCTGGGCTG
GGCTGGGCCAGGAATGCAGCGGGGCAGGGCTATTTAAGTCAAGGGCCGGCTGGCAACCCCAGCAAGCTG
TCCTGTGAGCAACCCAGCATGTCTGACATAGAAGAGGTGGTGGAAGAGTACGAGGAGGAGGAGCAGGAA
GAAGCAGCTGTTGAAGAAGAGGAGGACTGGAGAGAGGACGAAGACGAGCAGGAGGAGGCAGCGGAAGAG
GATGCTGAAGCAGAGGCTGAGACCGAGGAGACCAGGGCAGAAGAAGATGAAGAAGAAGAGGAAGCAAAG
GAGGCTGAAGATGGCCCAATGGAGGAGTCCAAACCAAAGCCCAGGTCGTTCATGCCCAACTTGGTGCCT
CCCAAGATCCCCGATGGAGAGAGAGTGGACTTTGATGACATCCACCGGAAGCGCATGGAGAAGGACCTG
AATGAGTTGCAGGCGCTGATCGAGGCTCACTTTGAGAACAGGAAGAAAGAGGAGGAGGAGCTCGTTTCT
CTCAAAGACAGGATCGAGAGACGTCGGGCAGAGCGGGCCGAGCAGCAGCGCATCCGGAATGAGCGGGAG
AAGGAGCGGCAGAACCGCCTGGCTGAAGAGAGGGCTCGACGAGAGGAGGAGGAGAACAGGAGGAAGGCT
GAGGATGAGGCCCGGAAGAAGAAGGCTTTGTCCAACATGATGCATTTTGGGGGTTACATCCAGAAGACA
GAGCGGAAAAGTGGGAAGAGGCAGACTGAGCGGGAAAAGAAGAAGAAGATTCTGGCTGAGAGGAGGAAG
GTGCTGGCCATTGACCACCTGAATGAAGATCAGCTGAGGGAGAAGGCCAAGGAGCTGTGGCAGAGCATC
TATAACTTGGAGGCAGAGAAGTTCGACCTGCAGGAGAAGTTCAAGCAGCAGAAATATGAGATCAATGTT
CTCCGAAACAGGATCAACGATAACCAGAAAGTCTCCAAGACCCGCGGGAAGGCTAAAGTCACCGGGCGC
TGGAAATAGtagtaatctagagcggccgcctcgaggagctccggccgCTGTGCCTTCTAGTTGCCAGCC
ATCTGTTGTTTGCCCCTCCCCCGTGCCTTCCTTGACCCTGGAAGGTGCCACTCCCACTGTCCTTTCCTA
ATAAAATGAGGAAATTGCATCaggaacccctagtgatggagttggccactccctctctgcgcgctcgct
cgctcactgaggccgggcgaccaaaggtcgcccgacgcccgggctttgcccgggcggcctcagtgagcg
agcgagcgcgcagagagggagtggccaa

In some embodiments, the promoter, Kozak sequence, and transgene may be assembled into an exemplary construct, wherein the exemplary construct comprises at least one promoter from Table B, at least one Kozak sequence from Table C, and at least one transgene from Table D. For example, a Desmin (Des1) promoter, an in silico derived Kozak Sequence, and TNNT2 may be placed within an exemplary construct.

TABLE B

| | |
|---|---|
| Promoter | Herpes Simplex virus (HSV) |
| | Thymidine kinase (TK) |
| | Rous Sarcoma Virus (RSV) |
| | Simian Virus 40 (SV40) |
| | Mouse Mammary Tumor Virus (MMTV) |
| | Ad E1A and cytomegalovirus (CMV) promoters |
| | chicken β-actin promoter (CBA) |
| | Desmin (Des1) |
| | Muscle Creatine Kinase (MCK) |
| | TNNT2 |

TABLE C

| | Native to TNNT2 |
|---|---|
| Kozak | Canonical Kozak (e.g., GCCACC |
| Sequence | (SEQ ID NO: 128)) |
| | In silico consensus (e.g., |
| | AGCCCCAAC (SEQ ID NO: 189)) |

TABLE D

| Transgene or | Human TNNT2 (e.g., comprising SEQ ID NOs: 7-27; |
|---|---|
| Protein to | SEQ ID NOs: 53-71; SEQ ID NOs: 78-95, SEQ ID |
| be Expressed | NOs: 104-121 arranged in sequence; SEQ ID NO: |
| | 129) |

### NON-LIMITING EMBODIMENTS

The following numerated Embodiments represent non-limiting aspects of the invention:
1. A nucleic acid comprising an expression cassette comprising a human TNNT2 coding sequence operably linked to a promoter and optionally an enhancer element, wherein the expression cassette is flanked on each side by an inverted terminal repeat sequence.
2. The nucleic acid of Embodiment 1 or 2, wherein the human TNNT2 coding sequence is codon-optimized for expression in human cells.
3. The nucleic acid of any one of Embodiments 1-3, wherein the human TNNT2 coding sequence has at least about 85% sequence identity to the sequence of SEQ ID NOs: 7-27; SEQ ID NOs: 53-71, SEQ ID NOs: 78-95, SEQ ID NOs: 104-121 arranged in sequence.
4. The nucleic acid of any one of Embodiments 1-4, wherein the promoter comprises a cardiac specific promoter.
5. The nucleic acid of any one of Embodiments 1-4, wherein the promoter is selected from the group consisting of: TNNT2, Desmin (DES1), and combinations thereof.
6. The nucleic acid of any one of Embodiments 1-5, wherein the promoter sequence has at least about 85% sequence identity to the sequence of SEQ ID NO: 3, 49, 77, or 102.
7. The nucleic acid of any one of Embodiments 1-6, wherein the expression cassette has at least about 85% sequence identity to the sequence of SEQ ID NOs: 1-31, SEQ ID NOs: 47-75, SEQ ID NOs: 76-98, SEQ ID NOs: 99-124, SEQ ID NOs: 125-132, or SEQ ID NOs: 133-141 arranged in sequence.
8. The nucleic acid of any one of Embodiments 1-6, wherein the expression cassette comprises the sequence of SEQ ID NO: 1-31, or SEQ ID NO: 47-75, arranged in sequence.
9. The nucleic acid of any one of Embodiments 1-8, wherein the nucleic acid is a recombinant adeno-associated virus (rAAV) vector.
10. The nucleic acid of Embodiment 9, wherein the nucleic acid is a single-stranded or self-complementary rAAV nucleic acid vector.
11. A recombinant adeno-associated virus (rAAV) particle comprising the nucleic acid of and one of Embodiments 1-10.
12. The rAAV particle of Embodiment 11, wherein the rAAV particle is an AAV9 particle.
13. The rAAV particle of Embodiment 11, wherein the rAAV particle is an AAVrh74 particle.
14. The rAAV particle of Embodiment 11, wherein the rAAV particle is an AAVrh10 particle.
15. A composition comprising a plurality of the rAAV particle of Embodiment 10, wherein the rAAV is selected from one or more of: AAV9 particles, AAVrh74 particles, and AAVrh10 particles.
16. The composition of Embodiment 15, further comprising a pharmaceutically acceptable carrier.
17. A method of treating dilated cardiomyopathy or hypertrophic cardiomyopathy, the method comprising:
   administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.
18. The method of Embodiment 17, wherein the rAAV is administered via intravenous injection.
19. The method of Embodiment 17 or 18, wherein between about 1×10¹³ and about 1×10¹⁴ rAAV vector genomes are administered.
20. A method of inducing increased expression of human TNNT2 in a target cell, comprising:
   contacting a target cell with a plurality of rAAV particles comprising a nucleic acid expression cassette comprising a functional human TNNT2 coding sequence operably linked to a promoter and optionally an enhancer element, wherein the expression construct is flanked on each side by an inverted terminal repeat sequence, and
   wherein said contacting results in the target cell increasing expression of functional human TNNT2 as compared to prior to the contacting, thereby increasing the expression of functional human TNNT2.
21. The method of Embodiment 20, wherein the contacting is in vivo.
22. The method of Embodiment 20 or 21, for the treatment of dilated cardiomyopathy or hypertrophic cardiomyopathy.
23. Use of the nucleic acid of any one of Embodiments 1-10, the rAAV particle of any one of Embodiments 11-14, or the composition of Embodiment 15 or 16 in the manufacture of a medicament for the treatment of dilated cardiomyopathy or hypertrophic cardiomyopathy.
24. Use of the nucleic acid of any one of Embodiments 1-10, the rAAV particle of any one of Embodiments 11-14, or the composition of Embodiment 15 or 16 for the treatment of dilated or hypertrophic cardiomyopathy.
25. The nucleic acid of the nucleic acid of any one of Embodiments 1-10, further comprising a silencing element, wherein the silencing element encodes an shRNA sequence.
26. A nucleic acid comprising an expression cassette comprising a human TNNT2 coding sequence operably linked to a promoter and optionally an enhancer element, wherein the expression cassette is flanked on each side by an inverted terminal repeat sequence.
27. A method of treating dilated cardiomyopathy or hypertrophic cardiomyopathy, the method comprising:
   administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.
28. The method of Embodiment 27, further comprising administering a therapeutically effective amount of a silencing construct.
29. A method of treating dilated cardiomyopathy or hypertrophic cardiomyopathy, the method comprising:
   administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.
30. The method of Embodiment 29, further comprising administering a therapeutically effective amount of a silencing construct.
31. A nucleic acid comprising an expression cassette comprising a human TNNT2 coding sequence operably linked to a promoter and optionally an enhancer element, wherein the expression cassette is flanked on each side by an inverted terminal repeat sequence.
32. The nucleic acid of Embodiment 31, wherein the human TNNT2 coding sequence is codon-optimized for expression in human cells.
33. The nucleic acid of Embodiment 31 or Embodiment 32, wherein the human TNNT2 coding sequence has at least about 85% sequence identity to the sequence of SEQ ID NOs: 7-27; SEQ ID NOs: 53-71; SEQ ID NOs: 78-95, SEQ ID NOs: 104-121 arranged in sequence.
34. The nucleic acid of any one of Embodiments 31 to 33, wherein the promoter comprises a cardiac specific promoter.
35. The nucleic acid of Embodiment 34, wherein the promoter is selected from the group consisting of: TNNT2, MHCK9 (MCK), Desmin (DES1), and combinations thereof.
36. The nucleic acid of Embodiment 34 or 35, wherein the promoter sequence has at least about 85% sequence identity to the sequence of SEQ ID NO: 3, 49, 77, or 102.
37. The nucleic acid of any one of Embodiments 31 to 36, wherein the expression cassette has at least about 85% sequence identity to the sequence of SEQ ID NOs: 1-31, or SEQ ID NOs: 47-75, or SEQ ID Nos: 76-98, or SEQ ID Nos: 99-124, or SEQ ID NOs: 125-132, or SEQ ID NOS: 133-141 arranged in sequence.
38. The nucleic acid of Embodiment 37, wherein the expression cassette comprises the sequence of SEQ ID NO: 1-31, or SEQ ID NO: 47-75, arranged in sequence.
39. The nucleic acid of any one of Embodiments 31 to 38, wherein the nucleic acid is a recombinant adeno-associated virus (rAAV) vector.
40. The nucleic acid of Embodiment 39, wherein the nucleic acid is a single-stranded or self-complementary rAAV nucleic acid vector.
41. A recombinant adeno-associated virus (rAAV) particle comprising the nucleic acid of Embodiment 39 or Embodiment 40.
42. The rAAV particle of Embodiment 41, wherein the rAAV particle is an AAV9 particle.
43. The rAAV particle of Embodiment 41, wherein the rAAV particle is an AAVrh74 particle.
44. The rAAV particle of Embodiment 41, wherein the rAAV particle is an AAVrh10 particle.
45. A composition comprising a plurality of the rAAV particle of any one of Embodiments 42, 43, or 44.
46. A method of inducing increased expression of human TNNT2 in a target cell, comprising:
   contacting a target cell with a plurality of rAAV particles comprising a nucleic acid expression cassette comprising a functional human TNNT2 coding sequence operably linked to a promoter and optionally an enhancer element, wherein the expression construct is flanked on each side by an inverted terminal repeat sequence, and
   wherein said contacting results in the target cell increasing expression of functional human TNNT2 as compared to prior to the contacting, thereby increasing the expression of functional human TNNT2.
47. The method of Embodiment 46, wherein the contacting is in vivo.
48. The method of Embodiment 46 or 47, for the treatment of dilated cardiomyopathy or hypertrophic cardiomyopathy.
49. Use of the nucleic acid of any one of Embodiments 31 to 39, the rAAV particle of any one of Embodiments 41 to 44, or the composition of Embodiment 45 in the manufacture of a medicament for the treatment of dilated cardiomyopathy or hypertrophic cardiomyopathy.
50. A nucleic acid comprising an expression construct comprising:
   a human TNNT2 coding sequence;
   a cardiac enhancer element operable linked to a promoter; and
   a Kozak sequence, wherein the Kozak sequence enhances transgene expression in the heart, wherein the expression construct is flanked on each side by an inverted terminal repeat sequence, wherein the Kozak sequence is non-native with respect to the human TNNT2 coding sequence, the cardiac enhancer element, and/or the promoter.
51. The nucleic acid of Embodiment 50, wherein the Kozak sequence is a synthetic sequence and has at least 85% sequence identity to the sequence of SEQ ID NO: 128.
52. The nucleic acid of Embodiment 50, wherein the Kozak sequence is a synthetic sequence and has at least 85% sequence identity to the sequence of SEQ ID NO: 189.

What is claimed is:

1. A nucleic acid comprising an expression cassette comprising a human TNNT2 coding sequence operably linked to a promoter and optionally an enhancer element, wherein the expression cassette is flanked on each side by an inverted terminal repeat sequence.

2. The nucleic acid of claim 1, wherein the human TNNT2 coding sequence is codon-optimized for expression in human cells.

3. The nucleic acid of claim 1, wherein the human TNNT2 coding sequence has at least about 85% sequence identity to the sequence of SEQ ID NOs: 7-27; SEQ ID NOs: 53-71; SEQ ID NOs: 78-95, SEQ ID NOs: 104-121 arranged in sequence.

4. The nucleic acid of claim 1, wherein the promoter comprises a cardiac specific promoter.

5. The nucleic acid of claim 4, wherein the promoter is selected from the group consisting of: TNNT2, Desmin (DES1), and combinations thereof.

6. The nucleic acid of claim 4, wherein the promoter sequence has at least about 85% sequence identity to the sequence of SEQ ID NO: 3, 49, 77, or 102.

7. The nucleic acid of claim 1, wherein the expression cassette has at least about 85% sequence identity to the sequence of SEQ ID NOs: 1-31, or SEQ ID NOs: 47-75, or SEQ ID NOs: 76-98, or SEQ ID NOs: 99-124, or SEQ ID NOs: 125-132, or SEQ ID NOs: 133-141 arranged in sequence.

8. The nucleic acid of claim 7, wherein the expression cassette comprises the sequence of SEQ ID NO: 1-31, or SEQ ID NO: 47-75, arranged in sequence.

9. The nucleic acid of claim 8, wherein the nucleic acid is a recombinant adeno-associated virus (rAAV) vector.

10. The nucleic acid of claim 9, wherein the nucleic acid is a single-stranded or self-complementary rAAV nucleic acid vector.

11. A recombinant adeno-associated virus (rAAV) particle comprising the nucleic acid of claim 10.

12. The rAAV particle of claim 11, wherein the rAAV particle is an AAV9 particle.

13. The rAAV particle of claim 11, wherein the rAAV particle is an AAVrh74 particle.

14. The rAAV particle of claim 11, wherein the rAAV particle is an AAVrh10 particle.

15. A composition comprising a plurality of the rAAV particle of claim 10, wherein the rAAV is selected from one or more of: AAV9 particles, AAVrh74 particles, and AAVrh10 particles.

16. The composition of claim 15, further comprising a pharmaceutically acceptable carrier.

17. A method of treating dilated cardiomyopathy or hypertrophic cardiomyopathy, the method comprising:
administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.

18. The method of claim 17, wherein the rAAV is administered via intravenous injection.

19. The method of claim 17, wherein between about 1×10¹³ and about 1×10¹⁴ rAAV vector genomes are administered.

20. A method of inducing increased expression of human TNNT2 in a target cell, comprising:
contacting a target cell with a plurality of rAAV particles comprising a nucleic acid expression cassette comprising a functional human TNNT2 coding sequence operably linked to a promoter and optionally an enhancer element, wherein the expression construct is flanked on each side by an inverted terminal repeat sequence, and
wherein said contacting results in the target cell increasing expression of functional human TNNT2 as compared to prior to the contacting, thereby increasing the expression of functional human TNNT2.

21. The method of claim 20, wherein the contacting is in vivo.

22. The method of claim 21, for the treatment of dilated cardiomyopathy or hypertrophic cardiomyopathy.

23. Use of the nucleic acid of claim 1, the rAAV particle of claim 11, or the composition of claim 15 in the manufacture of a medicament for the treatment of dilated cardiomyopathy or hypertrophic cardiomyopathy.

24. Use of the nucleic acid of claim 1, the rAAV particle of claim 11, or the composition of claim 15 for the treatment of dilated or hypertrophic cardiomyopathy.

25. The nucleic acid of claim 1, further comprising a silencing element, wherein the silencing element encodes an shRNA sequence.

26. A nucleic acid comprising an expression cassette comprising a human TNNT2 coding sequence operably linked to a promoter and optionally an enhancer element, wherein the expression cassette is flanked on each side by an inverted terminal repeat sequence.

27. A method of treating dilated cardiomyopathy or hypertrophic cardiomyopathy, the method comprising:
administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.

28. The method of claim 27, further comprising administering a therapeutically effective amount of a silencing construct.

29. A method of treating dilated cardiomyopathy or hypertrophic cardiomyopathy, the method comprising:
administering a therapeutically effective amount of rAAV comprising a nucleic acid expression construct comprising a human TNNT2 coding sequence operably linked to a promoter and optionally an enhancer element, wherein the expression construct is flanked on each side by an inverted terminal repeat sequence, and wherein said administration results in expression of a therapeutically effective amount of human TNNT2 thereby treating the dilated cardiomyopathy or hypertrophic cardiomyopathy.

30. The method of claim 29, further comprising administering a therapeutically effective amount of a silencing construct.

31. A nucleic acid comprising an expression cassette comprising a human TNNT2 coding sequence operably linked to a promoter and optionally an enhancer element, wherein the expression cassette is flanked on each side by an inverted terminal repeat sequence.

32. The nucleic acid of claim 31, wherein the human TNNT2 coding sequence is codon-optimized for expression in human cells.

33. The nucleic acid of claim 31 or claim 32, wherein the human TNNT2 coding sequence has at least about 85% sequence identity to the sequence of SEQ ID NOs: 7-27; SEQ ID NOs: 53-71; SEQ ID NOs: 78-95, SEQ ID NOs: 104-121 arranged in sequence.

34. The nucleic acid of any one of claims 31 to 33, wherein the promoter comprises a cardiac specific promoter.

35. The nucleic acid of claim 34, wherein the promoter is selected from the group consisting of: TNNT2, MHCK9 (MCK), Desmin (DES1), and combinations thereof.

36. The nucleic acid of claim 34 or 35, wherein the promoter sequence has at least about 85% sequence identity to the sequence of SEQ ID NO: 3, 49, 77, or 102.

37. The nucleic acid of any one of claims 31 to 36, wherein the expression cassette has at least about 85% sequence identity to the sequence of SEQ ID NOs: 1-31, or SEQ ID NOs: 47-75, or SEQ ID NOs: 76-98, or SEQ ID NOs: 99-124, or SEQ ID NOs: 125-132, or SEQ ID NOs: 133-141 arranged in sequence.

38. The nucleic acid of claim 37, wherein the expression cassette comprises the sequence of SEQ ID NOs: 1-31, or SEQ ID NOs: 47-75, arranged in sequence.

39. The nucleic acid of any one of claims 31 to 38, wherein the nucleic acid is a recombinant adeno-associated virus (rAAV) vector.

40. The nucleic acid of claim 39, wherein the nucleic acid is a single-stranded or self-complementary rAAV nucleic acid vector.

41. A recombinant adeno-associated virus (rAAV) particle comprising the nucleic acid of claim 39 or claim 40.

42. The rAAV particle of claim 41, wherein the rAAV particle is an AAV9 particle.

43. The rAAV particle of claim 41, wherein the rAAV particle is an AAVrh74 particle.

44. The rAAV particle of claim 41, wherein the rAAV particle is an AAVrh10 particle.

45. A composition comprising a plurality of the rAAV particle of any one of claim 42, 43, or 44.

46. A method of inducing increased expression of human TNNT2 in a target cell, comprising:
contacting a target cell with a plurality of rAAV particles comprising a nucleic acid expression cassette comprising a functional human TNNT2 coding sequence operably linked to a promoter and optionally an enhancer element, wherein the expression construct is flanked on each side by an inverted terminal repeat sequence, and
wherein said contacting results in the target cell increasing expression of functional human TNNT2 as compared to prior to the contacting, thereby increasing the expression of functional human TNNT2.

47. The method of claim 46, wherein the contacting is in vivo.

48. The method of claim 46 or 47, for the treatment of dilated cardiomyopathy or hypertrophic cardiomyopathy.

49. Use of the nucleic acid of any one of claims 31 to 39, the rAAV particle of any one of claims 41 to 44, or the composition of claim 45 in the manufacture of a medicament for the treatment of dilated cardiomyopathy or hypertrophic cardiomyopathy.

50. A nucleic acid comprising an expression construct comprising:
a human TNNT2 coding sequence;
a cardiac enhancer element operable linked to a promoter; and
a Kozak sequence, wherein the Kozak sequence enhances transgene expression in the heart, wherein the expression construct is flanked on each side by an inverted terminal repeat sequence, wherein the Kozak sequence is non-native with respect to the human TNNT2 coding sequence, the cardiac enhancer element, and/or the promoter.

51. The nucleic acid of claim 50, wherein the Kozak sequence is a synthetic sequence and has at least 85% sequence identity to the sequence of SEQ ID NO: 128.

52. The nucleic acid of claim 50, wherein the Kozak sequence is a synthetic sequence and has at least 85% sequence identity to the sequence of SEQ ID NO: 189.


SEQUENCE LISTING (213 sequences)

SEQ ID NO: 1 - DNA, 124 bp, synthetic construct
  source 1..124: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggcca

SEQ ID NO: 2 - DNA, 20 bp, synthetic construct
  source 1..20: mol_type = other DNA
tctagaggcgcgccaagctt

SEQ ID NO: 3 - DNA, 600 bp, synthetic construct
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgatcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 4 - DNA, 12 bp, synthetic construct
  source 1..12: mol_type = other DNA
agatctgaattc

SEQ ID NO: 5 - DNA, 147 bp, synthetic construct
  source 1..147: mol_type = other DNA
caggtaagtatcaaggttacaagacaggtttaaggagaccaatagaaactgggcttgtcgagacagagggccggccaagactcttgcgtttctgataggcacctattggtcttactgacatccactttgcctttctctccacagggt

SEQ ID NO: 6 - DNA, 12 bp, synthetic construct
  source 1..12: mol_type = other DNA
tctagaggtacc

SEQ ID NO: 7 - DNA, 14 bp, synthetic construct
  source 1..14: mol_type = other DNA
ggagagcagagacc

SEQ ID NO: 8 - DNA, 42 bp, synthetic construct
  source 1..42: mol_type = other DNA
atgtctgacatagaagaggtggtggaagagtacgaggaggag

SEQ ID NO: 9 - DNA, 265 bp, synthetic construct
  source 1..265: mol_type = other DNA
gtgagtatctggagcatcttgcctgagtggggtcctctcccgccgctgccctgacacctggtccaggagcctcccagctgtccctcggattctgggtagaagtagctgtgtgtgttttgggcaccccgaggagagatatagccatttcctttccactcctgtacccctgggttgtagatcccaggcagagcacaggccgagagacttcgggaagcacagaggttcagctccagggtcatcagtgtccttggccagtcttggctag

SEQ ID NO: 10 - skipped

SEQ ID NO: 11 - DNA, 265 bp, synthetic construct
  source 1..265: mol_type = other DNA
aagtgagcagaacgctggtgagaccaagctggctggggcggaggcttgcggaggatggtaggaggcagccctgcatgagaacggcaggccaggctagtgggtgtcattgcaaggtgggcagggcagcgtggactccactaggcaacaagggaaaagaaagggggattatctttggggaaaggccagtgtgtgcatgtgtgtgcaggcgtgtgtgtttgcatgtgcttgtgtgcgagctactgacagtgtttcctgttgctctcag

SEQ ID NO: 12 - skipped

SEQ ID NO: 13 - DNA, 15 bp, synthetic construct
  source 1..15: mol_type = other DNA
gaagcagctgttgaa

SEQ ID NO: 14 - DNA, 30 bp, synthetic construct
  source 1..30: mol_type = other DNA
gaagaggaggactggagagaggacgaagac

SEQ ID NO: 15 - DNA, 66 bp, synthetic construct
  source 1..66: mol_type = other DNA
gagcaggaggaggcagcggaagaggatgctgaagcagaggctgagaccgaggagaccagggcagaa

SEQ ID NO: 16 - DNA, 36 bp, synthetic construct
  source 1..36: mol_type = other DNA
gaagatgaagaagaagaggaagcaaaggaggctgaa

SEQ ID NO: 17 - DNA, 36 bp, synthetic construct
  source 1..36: mol_type = other DNA
gatggcccaatggaggagtccaaaccaaagcccagg

SEQ ID NO: 18 - DNA, 60 bp, synthetic construct
  source 1..60: mol_type = other DNA
tcgttcatgcccaacttggtgcctcccaagatccccgatggagagagagtggactttgat

SEQ ID NO: 19 - DNA, 120 bp, synthetic construct
  source 1..120: mol_type = other DNA
gacatccaccggaagcgcatggagaaggacctgaatgagttgcaggcgctgatcgaggctcactttgagaacaggaagaaagaggaggaggagctcgtttctctcaaagacaggatcgag

SEQ ID NO: 20 - DNA, 75 bp, synthetic construct
  source 1..75: mol_type = other DNA
agacgtcgggcagagcgggccgagcagcagcgcatccggaatgagcgggagaaggagcggcagaaccgcctggct

SEQ ID NO: 21 - DNA, 111 bp, synthetic construct
  source 1..111: mol_type = other DNA
gaagagagggctcgacgagaggaggaggagaacaggaggaaggctgaggatgaggcccggaagaagaaggctttgtccaacatgatgcattttgggggttacatccagaag

SEQ ID NO: 22 - DNA, 111 bp, synthetic construct
  source 1..111: mol_type = other DNA
acagagcggaaaagtgggaagaggcagactgagcgggaaaagaagaagaagattctggctgagaggaggaaggtgctggccattgaccacctgaatgaagatcagctgagg

SEQ ID NO: 23 - DNA, 90 bp, synthetic construct
  source 1..90: mol_type = other DNA
gagaaggccaaggagctgtggcagagcatctataacttggaggcagagaagttcgacctgcaggagaagttcaagcagcagaaatatgag

SEQ ID NO: 24 - DNA, 42 bp, synthetic construct
  source 1..42: mol_type = other DNA
atcaatgttctccgaaacaggatcaacgataaccagaaagtc

SEQ ID NO: 25 - DNA, 42 bp, synthetic construct
  source 1..42: mol_type = other DNA
tccaagacccgcgggaaggctaaagtcaccgggcgctggaaa

SEQ ID NO: 26 - skipped

SEQ ID NO: 27 - DNA, 196 bp, synthetic construct
  source 1..196: mol_type = other DNA
agcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactga

SEQ ID NO: 28 - DNA, 26 bp, synthetic construct
  source 1..26: mol_type = other DNA
gcggccgcctcgaggagctccggccg

SEQ ID NO: 29 - DNA, 112 bp, synthetic construct
  source 1..112: mol_type = other DNA
ctgtgccttctagttgccagccatctgttgtttgcccctcccccgtgccttccttgaccctggaaggtgccactcccactgtcctttcctaataaaatgaggaaattgcatc

SEQ ID NO: 30 - DNA, 25 bp, synthetic construct
  source 1..25: mol_type = other DNA
gcatgtcgacgcgccggcgtctaga

SEQ ID NO: 31 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 32 - protein, 14 aa, Homo sapiens
MSDIEEVVEEYEEE

SEQ ID NO: 33 - skipped

SEQ ID NO: 34 - protein, 5 aa, Homo sapiens
EAAVE

SEQ ID NO: 35 - protein, 10 aa, Homo sapiens
EEEDWREDED

SEQ ID NO: 36 - protein, 22 aa, Homo sapiens
EQEEAAEEDAEAEAETEETRAE

SEQ ID NO: 37 - protein, 12 aa, Homo sapiens
EDEEEEEAKEAE

SEQ ID NO: 38 - protein, 12 aa, Homo sapiens
DGPMEESKPKPR

SEQ ID NO: 39 - protein, 20 aa, Homo sapiens
SFMPNLVPPKIPDGERVDFD

SEQ ID NO: 40 - protein, 40 aa, Homo sapiens
DIHRKRMEKDLNELQALIEAHFENRKKEEEELVSLKDRIE

SEQ ID NO: 41 - protein, 25 aa, Homo sapiens
RRRAERAEQQRIRNEREKERQNRLA

SEQ ID NO: 42 - protein, 37 aa, Homo sapiens
EERARREEEENRRKAEDEARKKKALSNMMHFGGYIQK

SEQ ID NO: 43 - protein, 37 aa, Homo sapiens
TERKSGKRQTEREKKKKILAERRKVLAIDHLNEDQLR

SEQ ID NO: 44 - protein, 30 aa, Homo sapiens
EKAKELWQSIYNLEAEKFDLQEKFKQQKYE

SEQ ID NO: 45 - protein, 14 aa, Homo sapiens
INVLRNRINDNQKV

SEQ ID NO: 46 - protein, 14 aa, Homo sapiens
SKTRGKAKVTGRWK

SEQ ID NO: 47 - DNA, 124 bp, synthetic construct; identical to SEQ ID NO: 1
  source 1..124: mol_type = other DNA

SEQ ID NO: 48 - DNA, 20 bp, synthetic construct
  source 1..20: mol_type = other DNA
tctagaggcgcgccaagctt

SEQ ID NO: 49 - DNA, 600 bp, synthetic construct; identical to SEQ ID NO: 3
  source 1..600: mol_type = other DNA

SEQ ID NO: 50 - DNA, 12 bp, synthetic construct
  source 1..12: mol_type = other DNA
agatctgaattc

SEQ ID NO: 51 - DNA, 147 bp, synthetic construct; identical to SEQ ID NO: 5
  source 1..147: mol_type = other DNA

SEQ ID NO: 52 - DNA, 12 bp, synthetic construct
  source 1..12: mol_type = other DNA
tctagaggtacc

SEQ ID NO: 53 - DNA, 14 bp, synthetic construct
  source 1..14: mol_type = other DNA
ggagagcagagacc

SEQ ID NO: 54 - DNA, 42 bp, synthetic construct; identical to SEQ ID NO: 8
  source 1..42: mol_type = other DNA

SEQ ID NO: 55 - DNA, 530 bp, synthetic construct
  source 1..530: mol_type = other DNA
gtgagtatctggagcatcttgcctgagtggggtcctctcccgccgctgccctgacacctggtccaggagcctcccagctgtccctcggattctgggtagaagtagctgtgtgtgttttgggcaccccgaggagagatatagccatttcctttccactcctgtacccctgggttgtagatcccaggcagagcacaggccgagagacttcgggaagcacagaggttcagctccagggtcatcagtgtccttggccagtcttggctagaagtgagcagaacgctggtgagaccaagctggctggggcggaggcttgcggaggatggtaggaggcagccctgcatgagaacggcaggccaggctagtgggtgtcattgcaaggtgggcagggcagcgtggactccactaggcaacaagggaaaagaaagggggattatctttggggaaaggccagtgtgtgcatgtgtgtgcaggcgtgtgtgtttgcatgtgcttgtgtgcgagctactgacagtgtttcctgttgctctcag

SEQ ID NO: 56 - skipped

SEQ ID NO: 57 - DNA, 15 bp, synthetic construct
  source 1..15: mol_type = other DNA
gaagcagctgttgaa

SEQ ID NO: 58 - DNA, 30 bp, synthetic construct; identical to SEQ ID NO: 14
  source 1..30: mol_type = other DNA

SEQ ID NO: 59 - DNA, 66 bp, synthetic construct; identical to SEQ ID NO: 15
  source 1..66: mol_type = other DNA

SEQ ID NO: 60 - DNA, 36 bp, synthetic construct; identical to SEQ ID NO: 16
  source 1..36: mol_type = other DNA

SEQ ID NO: 61 - DNA, 36 bp, synthetic construct; identical to SEQ ID NO: 17
  source 1..36: mol_type = other DNA

SEQ ID NO: 62 - DNA, 60 bp, synthetic construct; identical to SEQ ID NO: 18
  source 1..60: mol_type = other DNA

SEQ ID NO: 63 - DNA, 120 bp, synthetic construct; identical to SEQ ID NO: 19
  source 1..120: mol_type = other DNA

SEQ ID NO: 64 - DNA, 75 bp, synthetic construct; identical to SEQ ID NO: 20
  source 1..75: mol_type = other DNA

SEQ ID NO: 65 - DNA, 111 bp, synthetic construct; identical to SEQ ID NO: 21
  source 1..111: mol_type = other DNA

SEQ ID NO: 66 - DNA, 111 bp, synthetic construct; identical to SEQ ID NO: 22
  source 1..111: mol_type = other DNA

SEQ ID NO: 67 - DNA, 90 bp, synthetic construct; identical to SEQ ID NO: 23
  source 1..90: mol_type = other DNA

SEQ ID NO: 68 - DNA, 42 bp, synthetic construct; identical to SEQ ID NO: 24
  source 1..42: mol_type = other DNA

SEQ ID NO: 69 - DNA, 42 bp, synthetic construct; identical to SEQ ID NO: 25
  source 1..42: mol_type = other DNA

SEQ ID NO: 70 - skipped

SEQ ID NO: 71 - DNA, 196 bp, synthetic construct; identical to SEQ ID NO: 27
  source 1..196: mol_type = other DNA

SEQ ID NO: 72 - DNA, 26 bp, synthetic construct
  source 1..26: mol_type = other DNA
gcggccgcctcgaggagctccggccg

SEQ ID NO: 73 - DNA, 112 bp, synthetic construct; identical to SEQ ID NO: 29
  source 1..112: mol_type = other DNA

SEQ ID NO: 74 - DNA, 25 bp, synthetic construct
  source 1..25: mol_type = other DNA
gcatgtcgacgcgccggcgtctaga

SEQ ID NO: 75 - DNA, 145 bp, synthetic construct; identical to SEQ ID NO: 31
  source 1..145: mol_type = other DNA

SEQ ID NO: 76 - DNA, 124 bp, synthetic construct; identical to SEQ ID NO: 1
  source 1..124: mol_type = other DNA

SEQ ID NO: 77 - DNA, 449 bp, synthetic construct
  source 1..449: mol_type = other DNA
tagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaa

SEQ ID NO: 78 - DNA, 14 bp, synthetic construct
  source 1..14: mol_type = other DNA
ggagagcagagacc

SEQ ID NO: 79 - DNA, 42 bp, synthetic construct; identical to SEQ ID NO: 8
  source 1..42: mol_type = other DNA

SEQ ID NO: 80 - DNA, 265 bp, synthetic construct; identical to SEQ ID NO: 9
  source 1..265: mol_type = other DNA

SEQ ID NO: 81 - DNA, 265 bp, synthetic construct; identical to SEQ ID NO: 11
  source 1..265: mol_type = other DNA

SEQ ID NO: 82 - skipped

SEQ ID NO: 83 - DNA, 15 bp, synthetic construct
  source 1..15: mol_type = other DNA
gaagcagctgttgaa

SEQ ID NO: 84 - DNA, 30 bp, synthetic construct; identical to SEQ ID NO: 14
  source 1..30: mol_type = other DNA

SEQ ID NO: 85 - DNA, 66 bp, synthetic construct; identical to SEQ ID NO: 15
  source 1..66: mol_type = other DNA

SEQ ID NO: 86 - DNA, 36 bp, synthetic construct; identical to SEQ ID NO: 16
  source 1..36: mol_type = other DNA

SEQ ID NO: 87 - DNA, 36 bp, synthetic construct; identical to SEQ ID NO: 17
  source 1..36: mol_type = other DNA

SEQ ID NO: 88 - DNA, 60 bp, synthetic construct; identical to SEQ ID NO: 18
  source 1..60: mol_type = other DNA

SEQ ID NO: 89 - DNA, 120 bp, synthetic construct; identical to SEQ ID NO: 19
  source 1..120: mol_type = other DNA

SEQ ID NO: 90 - DNA, 75 bp, synthetic construct; identical to SEQ ID NO: 20
  source 1..75: mol_type = other DNA

SEQ ID NO: 91 - DNA, 111 bp, synthetic construct; identical to SEQ ID NO: 21
  source 1..111: mol_type = other DNA

SEQ ID NO: 92 - DNA, 111 bp, synthetic construct; identical to SEQ ID NO: 22
  source 1..111: mol_type = other DNA

SEQ ID NO: 93 - DNA, 90 bp, synthetic construct; identical to SEQ ID NO: 23
  source 1..90: mol_type = other DNA

SEQ ID NO: 94 - DNA, 42 bp, synthetic construct; identical to SEQ ID NO: 24
  source 1..42: mol_type = other DNA

SEQ ID NO: 95 - DNA, 45 bp, synthetic construct
  source 1..45: mol_type = other DNA
tccaagacccgcgggaaggctaaagtcaccgggcgctggaaatag

SEQ ID NO: 96 - DNA, 196 bp, synthetic construct; identical to SEQ ID NO: 27
  source 1..196: mol_type = other DNA

SEQ ID NO: 97 - DNA, 49 bp, synthetic construct
  source 1..49: mol_type = other DNA
aataaaagatccttattttcattggatctgtgtgttggttttttgtgtg

SEQ ID NO: 98 - DNA, 145 bp, synthetic construct; identical to SEQ ID NO: 31
  source 1..145: mol_type = other DNA

SEQ ID NO: 99 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 100 - DNA, 191 bp, synthetic construct
  source 1..191: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgt

SEQ ID NO: 101 - DNA, 111 bp, synthetic construct
  source 1..111: mol_type = other DNA
ctgcccatgtaaggaggcaaggcctggggacacccgagatgcctggttataattaacccagacatgtggctgcccccccccccccaacacctgctgcctctaaaaataacc

SEQ ID NO: 102 - DNA, 265 bp, synthetic construct
  source 1..265: mol_type = other DNA
gttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctc

SEQ ID NO: 103 - DNA, 43 bp, synthetic construct
  source 1..43: mol_type = other DNA
accaccacctccacagcacagacagacactcaggagcagccag

SEQ ID NO: 104 - DNA, 14 bp, synthetic construct
  source 1..14: mol_type = other DNA
ggagagcagagacc

SEQ ID NO: 105 - DNA, 42 bp, synthetic construct; identical to SEQ ID NO: 8
  source 1..42: mol_type = other DNA

SEQ ID NO: 106 - DNA, 50 bp, synthetic construct
  source 1..50: mol_type = other DNA
gtgagtatctggagcatcttgcctgagtggggtcctctcccgccgctgcc

SEQ ID NO: 107 - DNA, 50 bp, synthetic construct
  source 1..50: mol_type = other DNA
ttgcatgtgcttgtgtgcgagctactgacagtgtttcctgttgctctcag

SEQ ID NO: 108 - skipped

SEQ ID NO: 109 - DNA, 15 bp, synthetic construct
  source 1..15: mol_type = other DNA
gaagcagctgttgaa

SEQ ID NO: 110 - DNA, 30 bp, synthetic construct; identical to SEQ ID NO: 14
  source 1..30: mol_type = other DNA

SEQ ID NO: 111 - DNA, 66 bp, synthetic construct; identical to SEQ ID NO: 15
  source 1..66: mol_type = other DNA

SEQ ID NO: 112 - DNA, 36 bp, synthetic construct; identical to SEQ ID NO: 16
  source 1..36: mol_type = other DNA

SEQ ID NO: 113 - DNA, 36 bp, synthetic construct; identical to SEQ ID NO: 17
  source 1..36: mol_type = other DNA

SEQ ID NO: 114 - DNA, 60 bp, synthetic construct; identical to SEQ ID NO: 18
  source 1..60: mol_type = other DNA

SEQ ID NO: 115 - DNA, 120 bp, synthetic construct; identical to SEQ ID NO: 19
  source 1..120: mol_type = other DNA

SEQ ID NO: 116 - DNA, 75 bp, synthetic construct; identical to SEQ ID NO: 20
  source 1..75: mol_type = other DNA

SEQ ID NO: 117 - DNA, 111 bp, synthetic construct; identical to SEQ ID NO: 21
  source 1..111: mol_type = other DNA

SEQ ID NO: 118 - DNA, 111 bp, synthetic construct; identical to SEQ ID NO: 22
  source 1..111: mol_type = other DNA

SEQ ID NO: 119 - DNA, 90 bp, synthetic construct; identical to SEQ ID NO: 23
  source 1..90: mol_type = other DNA

SEQ ID NO: 120 - DNA, 42 bp, synthetic construct; identical to SEQ ID NO: 24
  source 1..42: mol_type = other DNA

SEQ ID NO: 121 - DNA, 45 bp, synthetic construct; identical to SEQ ID NO: 95
  source 1..45: mol_type = other DNA

SEQ ID NO: 122 - DNA, 196 bp, synthetic construct; identical to SEQ ID NO: 27
  source 1..196: mol_type = other DNA

SEQ ID NO: 123 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 124 - DNA, 145 bp, synthetic construct; identical to SEQ ID NO: 31
  source 1..145: mol_type = other DNA

SEQ ID NO: 125 - DNA, 124 bp, synthetic construct; identical to SEQ ID NO: 1
  source 1..124: mol_type = other DNA

SEQ ID NO: 126 - DNA, 62 bp, synthetic construct
  source 1..62: mol_type = other DNA
cctagttgttaatagtaatcaattacggggtcattagttcatagcccatatatggagttccg

SEQ ID NO: 127 - DNA, 687 bp, synthetic construct
  source 1..687: mol_type = other DNA
ccctgcccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctaatcagaccctttctggaaatcagcccactgtttatatacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtca

SEQ ID NO: 128 - skipped

SEQ ID NO: 129 - DNA, 888 bp, synthetic construct
  source 1..888: mol_type = other DNA
atgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatag

SEQ ID NO: 130 - DNA, 38 bp, synthetic construct
  source 1..38: mol_type = other DNA
tagtaatctagagcggccgcctcgaggagctccggccg

SEQ ID NO: 131 - DNA, 112 bp, synthetic construct; identical to SEQ ID NO: 29
  source 1..112: mol_type = other DNA

SEQ ID NO: 132 - DNA, 145 bp, synthetic construct; identical to SEQ ID NO: 31
  source 1..145: mol_type = other DNA

SEQ ID NO: 133 - DNA, 124 bp, synthetic construct; identical to SEQ ID NO: 1
  source 1..124: mol_type = other DNA

SEQ ID NO: 134 - DNA, 62 bp, synthetic construct; identical to SEQ ID NO: 126
  source 1..62: mol_type = other DNA

SEQ ID NO: 135 - DNA, 687 bp, synthetic construct; identical to SEQ ID NO: 127
  source 1..687: mol_type = other DNA

SEQ ID NO: 136 - skipped

SEQ ID NO: 137 - DNA, 888 bp, synthetic construct; identical to SEQ ID NO: 129
  source 1..888: mol_type = other DNA

SEQ ID NO: 138 - DNA, 196 bp, synthetic construct; identical to SEQ ID NO: 27
  source 1..196: mol_type = other DNA

SEQ ID NO: 139 - DNA, 38 bp, synthetic construct; identical to SEQ ID NO: 130
  source 1..38: mol_type = other DNA

SEQ ID NO: 140 - DNA, 112 bp, synthetic construct; identical to SEQ ID NO: 29
  source 1..112: mol_type = other DNA

SEQ ID NO: 141 - DNA, 145 bp, synthetic construct; identical to SEQ ID NO: 31
  source 1..145: mol_type = other DNA

SEQ ID NO: 142 - DNA, 124 bp, synthetic construct; identical to SEQ ID NO: 1
  source 1..124: mol_type = other DNA

SEQ ID NO: 143 - DNA, 62 bp, synthetic construct; identical to SEQ ID NO: 126
  source 1..62: mol_type = other DNA

SEQ ID NO: 144 - DNA, 687 bp, synthetic construct; identical to SEQ ID NO: 127
  source 1..687: mol_type = other DNA

SEQ ID NO: 145 - DNA, 259 bp, synthetic construct
  source 1..259: mol_type = other DNA
gtaagcgcccctaaaatccctttggcacaatgtctcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccag

SEQ ID NO: 146 - skipped

SEQ ID NO: 147 - DNA, 888 bp, synthetic construct; identical to SEQ ID NO: 129
  source 1..888: mol_type = other DNA

SEQ ID NO: 148 - DNA, 196 bp, synthetic construct; identical to SEQ ID NO: 27
  source 1..196: mol_type = other DNA

SEQ ID NO: 149 - DNA, 38 bp, synthetic construct; identical to SEQ ID NO: 130
  source 1..38: mol_type = other DNA

SEQ ID NO: 150 - DNA, 112 bp, synthetic construct; identical to SEQ ID NO: 29
  source 1..112: mol_type = other DNA

SEQ ID NO: 151 - DNA, 145 bp, synthetic construct; identical to SEQ ID NO: 31
  source 1..145: mol_type = other DNA

SEQ ID NO: 152 - DNA, 124 bp, synthetic construct; identical to SEQ ID NO: 1
  source 1..124: mol_type = other DNA

SEQ ID NO: 153 - DNA, 62 bp, synthetic construct; identical to SEQ ID NO: 126
  source 1..62: mol_type = other DNA

SEQ ID NO: 154 - DNA, 687 bp, synthetic construct; identical to SEQ ID NO: 127
  source 1..687: mol_type = other DNA

SEQ ID NO: 155 - DNA, 259 bp, synthetic construct; identical to SEQ ID NO: 145
  source 1..259: mol_type = other DNA

SEQ ID NO: 156 - skipped

SEQ ID NO: 157 - DNA, 888 bp, synthetic construct; identical to SEQ ID NO: 129
  source 1..888: mol_type = other DNA

SEQ ID NO: 158 - DNA, 38 bp, synthetic construct; identical to SEQ ID NO: 130
  source 1..38: mol_type = other DNA

SEQ ID NO: 159 - DNA, 112 bp, synthetic construct; identical to SEQ ID NO: 29
  source 1..112: mol_type = other DNA

SEQ ID NO: 160 - DNA, 145 bp, synthetic construct; identical to SEQ ID NO: 31
  source 1..145: mol_type = other DNA

SEQ ID NO: 161 - DNA, 124 bp, synthetic construct; identical to SEQ ID NO: 1
  source 1..124: mol_type = other DNA

SEQ ID NO: 162 - DNA, 200 bp, synthetic construct
  source 1..200: mol_type = other DNA
tagaacgtttctaagtttttccattcagttaaaccgactaaacttttctgcttgaagttttaatgaattgttattaacacccagatattgttttagaaaaattattcaaaagcattcacaaccaacttatcgggactagccaaattgatcatcaagtttatataataaaatactaaaaatttagtcttacattatggaat

SEQ ID NO: 163 - DNA, 61 bp, synthetic construct
  source 1..61: mol_type = other DNA
ctagttgttaatagtaatcaattacggggtcattagttcatagcccatatatggagttccg

SEQ ID NO: 164 - DNA, 687 bp, synthetic construct; identical to SEQ ID NO: 127
  source 1..687: mol_type = other DNA

SEQ ID NO: 165 - skipped

SEQ ID NO: 166 - DNA, 888 bp, synthetic construct; identical to SEQ ID NO: 129
  source 1..888: mol_type = other DNA

SEQ ID NO: 167 - DNA, 38 bp, synthetic construct; identical to SEQ ID NO: 130
  source 1..38: mol_type = other DNA

SEQ ID NO: 168 - DNA, 112 bp, synthetic construct; identical to SEQ ID NO: 29
  source 1..112: mol_type = other DNA

SEQ ID NO: 169 - DNA, 145 bp, synthetic construct; identical to SEQ ID NO: 31
  source 1..145: mol_type = other DNA

SEQ ID NO: 170 - DNA, 124 bp, synthetic construct; identical to SEQ ID NO: 1
  source 1..124: mol_type = other DNA

SEQ ID NO: 171 - DNA, 62 bp, synthetic construct; identical to SEQ ID NO: 126
  source 1..62: mol_type = other DNA

SEQ ID NO: 172 - DNA, 687 bp, synthetic construct; identical to SEQ ID NO: 127
  source 1..687: mol_type = other DNA

SEQ ID NO: 173 - skipped

SEQ ID NO: 174 - DNA, 888 bp, synthetic construct
  source 1..888: mol_type = other DNA
atgagcgacatcgaggaggtggtggaagagtacgaggaagaggaacaggaggaagccgccgtggaagaagaagaggattggcgggaagatgaggatgaacaggaggaagctgctgaggaggacgccgaggccgaggcagaaaccgaggagacaagagccgaagaagatgaggaggaggaagaggccaaagaggccgaggacggccccatggaagagtccaagcctaagcccagaagcttcatgcctaacctggtcccacctaagattcctgatggagaacgggtggacttcgacgacatccaccggaagcggatggaaaaagacctgaacgagctgcaggccctgatcgaggcccacttcgagaatcggaagaaggaagaagaggagctcgtgtctctgaaggacagaatcgagcggagaagagccgaacgggccgagcaacaaagaatcagaaacgagcgcgagaaggagagacagaacagactggccgaggagagagctagaagagaagaagaggaaaaccggaggaaggccgaggacgaggccagaaagaaaaaggccctgagcaacatgatgcatttcggcggctacatccagaagacagagcggaaaagcggcaaaagacagaccgagcgtgagaaaaagaagaagatcctggctgagcggagaaaagtgctggccatcgaccacctgaatgaggatcagctgagagaaaaggccaaggaactgtggcagagcatctacaacctggaagctgaaaagtttgacctgcaggagaagttcaagcagcagaagtatgagatcaacgtgctgaggaatagaatcaacgacaaccagaaggtttctaagaccagaggcaaggccaaggtgaccggcagatggaagtga

SEQ ID NO: 175 - DNA, 196 bp, synthetic construct; identical to SEQ ID NO: 27
  source 1..196: mol_type = other DNA

SEQ ID NO: 176 - DNA, 38 bp, synthetic construct; identical to SEQ ID NO: 130
  source 1..38: mol_type = other DNA

SEQ ID NO: 177 - DNA, 112 bp, synthetic construct; identical to SEQ ID NO: 29
  source 1..112: mol_type = other DNA

SEQ ID NO: 178 - DNA, 145 bp, synthetic construct; identical to SEQ ID NO: 31
  source 1..145: mol_type = other DNA

SEQ ID NO: 179 - DNA, 124 bp, synthetic construct; identical to SEQ ID NO: 1
  source 1..124: mol_type = other DNA

SEQ ID NO: 180 - DNA, 62 bp, synthetic construct; identical to SEQ ID NO: 126
  source 1..62: mol_type = other DNA

SEQ ID NO: 181 - DNA, 687 bp, synthetic construct; identical to SEQ ID NO: 127
  source 1..687: mol_type = other DNA

SEQ ID NO: 182 - skipped

SEQ ID NO: 183 - DNA, 45 bp, synthetic construct
  source 1..45: mol_type = other DNA
atgatgtctgacatagaagaggtggtggaagagtacgaggaggag

SEQ ID NO: 184 - DNA, 259 bp, synthetic construct; identical to SEQ ID NO: 145
  source 1..259: mol_type = other DNA

SEQ ID NO: 185 - DNA, 846 bp, synthetic construct
  source 1..846: mol_type = other DNA
gagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatag

SEQ ID NO: 186 - DNA, 38 bp, synthetic construct; identical to SEQ ID NO: 130
  source 1..38: mol_type = other DNA

SEQ ID NO: 187 - DNA, 112 bp, synthetic construct; identical to SEQ ID NO: 29
  source 1..112: mol_type = other DNA

SEQ ID NO: 188 - DNA, 145 bp, synthetic construct; identical to SEQ ID NO: 31
  source 1..145: mol_type = other DNA

SEQ ID NO: 189-204 - skipped (16 entries)

SEQ ID NO: 205 - DNA, 1615 bp, synthetic construct
  source 1..1615: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagcgccaccatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 206 - DNA, 1615 bp, synthetic construct
  source 1..1615: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagccccaacatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 207 - DNA, 1615 bp, synthetic construct
  source 1..1615: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgcaacccagcatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 208 - DNA, 1692 bp, synthetic construct
  source 1..1692: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagcgccaccatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 209 - DNA, 1692 bp, synthetic construct
  source 1..1692: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagccccaacatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 210 - DNA, 1691 bp, synthetic construct
  source 1..1691: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctccaacccagcatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 211 - DNA, 1478 bp, synthetic construct
  source 1..1478: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagagcgccaccatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 212 - DNA, 1478 bp, synthetic construct
  source 1..1478: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagagccccaacatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 213 - DNA, 1478 bp, synthetic construct
  source 1..1478: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagcaacccagcatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagtagtaatctagagcggccgcctcgaggagctccggccgctgtgccttctagttgccagccatctgttgtttgcccctcccccgtgccttccttgaccctggaaggtgccactcccactgtcctttcctaataaaatgaggaaattgcatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa